(19) United States
(12) Patent Application Publication
(10) Pub. No.: US 2010/0239592 A1
(43) Pub. Date: Sep. 23, 2010

(54) METHODS OF ANALYSIS AND DETECTING MOLECULAR DEFECTS IN TRANSCOBALAMIN I DEFICIENCY

(75) Inventors: RALPH CARMEL, NEW YORK, NY (US); Zvi Kelman, Gaithersburg, MD (US)

Correspondence Address: BAKER BOTTS L.L.P., 30 ROCKEFELLER PLAZA, 44TH FLOOR, NEW YORK, NY, 10112-4498, US

(73) Assignee: NEW YORK METHODIST HOSPITAL, BROOKLYN, NY (US)

(21) Appl. No.: 12/706,619

(22) Filed: Feb. 16, 2010

Related U.S. Application Data
(60) Provisional application No. 61/152,534, filed on Feb. 13, 2009.
(60) Provisional application No. 61/218,273, filed on Jun. 18, 2009.

(51) Int. Cl. A61K 39/395; C12Q 1/68; G01N 33/00; C12Q 1/02; C40B 30/00; A61K 31/714; A61K 38/45; A61K 31/713; A61P 7/00

(52) U.S. Cl. 424/158.1; 435/6; 436/86; 435/29; 506/7; 514/52; 514/44 A; 424/94.5; 514/44 R

(57) ABSTRACT

The present invention provides methods, compositions, and kits for detecting TC I deficiency in individuals with low serum cobalamin levels. Methods for detecting TCN1 mutations and/or the TC I isoforms or products of TCN1 mutations are useful for detecting and diagnosing those who have TC I deficiency and thereby can be said to have TC I deficiency instead of cobalamin deficiency.

### CROSS-REFERENCE TO RELATED APPLICATION

The present application claims the benefit of U.S. Provisional Application No. 61/152,534, filed Feb. 13, 2009, and U.S. Provisional Application No. 61/218,273, filed Jun. 18, 2009, each of which is hereby incorporated by reference in their entireties.

### GRANT INFORMATION

The invention disclosed herein was made with United States Government support under National Institute of Health Grant DK32640. Accordingly, the U.S. Government has certain rights herein.

### FIELD OF THE INVENTION

The present invention relates to methods, compositions, and kits for detecting heritable transcobalamin I (TC I) deficiency. Methods for detecting mutations in the gene encoding transcobalamin I (TCN1) are useful for detecting and diagnosing individuals who have or are at risk of TC I deficiency. Methods of detecting TC I isoforms (the protein products of a mutated form of TCN1) can also be useful in this role. These methods and compositions will be useful in the differential diagnosis and correct treatment of persons with low serum levels of cobalamin (vitamin B₁₂), which are usually assumed to represent cobalamin deficiency but are often caused by TC I deficiency instead. The functions of TC I are unclear but may become better understood, in part by application of the molecular TC I techniques described here. Methods for detecting and diagnosing TC I deficiency may also be useful in differentiating it from cobalamin deficiency or the prophylaxis or treatment of patients with TC I deficiency.

### BACKGROUND OF THE INVENTION

Traditional methods for screening for heritable diseases have depended on either the identification of abnormal gene products (e.g., sickle cell anemia) or an abnormal phenotype (e.g., mental retardation). These methods can also be applied to heritable conditions with late onset of phenotypic manifestations such as, for example, vascular disease, but are not often applied to disorders that remain phenotypically obscure unless a manifestation is recognized in the course of considering other diseases that can be confused with it. TC I deficiency is such a condition and is clinically important because of the diagnostic problems its recognition poses.

Transcobalamin I (TC I; also known as haptocorrin, cobalophilin, alpha-globulin binder, or R binder) is distinct from other cobalamin-binding proteins (see below) but has areas of structural homology to them. It is a glycoprotein of granulocytic and glandular epithelial cell origins and a major protein constituent of secondary granules in neutrophils. TC I is the least understood of the three cobalamin-binding proteins in humans; the function of TC I is still unknown. Deficiency of TC I, originally thought to be rare with very few cases identified over many decades (Carmel, 1969; Carmel, 1982; Jenks et al., 1983; Carmel, 1983; and Zittoun et al., 1988), is now believed to be more common (Carmel, 2003). Unlike deficiencies of the other two specific cobalamin-binding proteins, transcobalamin II (TC II) and intrinsic factor, definitive clinical sequelae of TCI deficiency are not currently known, except for a low serum cobalamin level, which occurs because >75% of cobalamin normally circulates in the blood stream attached to TC I (Carmel, 1981). As a result of absent or reduced TC I, serum cobalamin is decreased because it can then be carried only as holo-TC II (TC II with attached cobalamin), which is rapidly cleared from the blood and maintains normal cobalamin delivery to tissues despite low serum cobalamin (Carmel, 1969).

The function of TC I is not clear and no specific receptors for TC I are known to exist (Carmel, 1981), but possible roles in humans have been proposed. One proposed role is binding of nonfunctional, and sometimes potentially harmful, cobalamin analogues as well as functional cobalamin, thus preventing access of the analogues to cells (Kondo et al, 1980); TC II, in contrast, binds only functional cobalamin and delivers it to cells. Another proposed role for TC I is binding cobalamin and rendering it unavailable to microbes (Gullberg, 1974). Finally, desialylated TC I may contribute to delivery of cobalamin to liver via nonspecific asialoglycoprotein receptors, leading to biliary excretion (Burger et al, 1975).

The clinical importance of determining whether a patient suffers from TC I deficiency arises because patients with primary cobalamin deficiency and those with TC I deficiency both exhibit low serum cobalamin levels. This phenomenon confuses the clinical diagnosis, and therefore also the management, of primary cobalamin deficiency. This can have serious consequences because primary cobalamin deficiency, for example, cobalamin deficiency resulting from reduced dietary intake of cobalamin, pernicious anemia, or malabsorption of cobalamin, has major clinical complications such as neurological dysfunction (ranging from mild sensory neuropathy to severe spinal cord degeneration), cognitive and psychiatric disturbances, painful glossitis, and anemia that can be very severe and include pancytopenia. As such, true cobalamin deficiency requires prompt and often lifelong cobalamin therapy, whereas low cobalamin levels due to TC I deficiency do not.

TC I deficiency is currently difficult to diagnose. Few laboratories measure plasma TC I protein levels, and most methods often fail to differentiate between TC I and TC II. In U.S. Pat. No. 4,167,556, Selhub et al. describe a process for determining TCI, TCII, and “TCIII” (a partially desialylated isoprotein with amino acid identity to TC I) levels in serum. In U.S. Pat. No. 4,680,273, Herbert describes assaying for cobalamin deficiency by determining whether essentially all of the cobalamin is carried by a combination of TCI and TCIII and/or if essentially no cobalamin is carried by TC II in a blood sample.

In available tests that do differentiate between TC I and TC II, the assays often measure only the ability to bind added cobalamin, thus detecting only apo-TC (i.e., TC I not carrying endogenously attached cobalamin), which is the less abundant state of TC I in the blood. The methods are also subject to considerable pre-analytic and analytic artifact leading to misdiagnoses of cobalamin deficiency as TC I deficiency, and vice versa (Adcock and McKnight 2002). A few research laboratories perform radioimmunoassay (RIA), which provides the only currently reliable, direct method to identify and quantitate total TC I protein levels, and not just apo-TC I (Carmel 2003). The TC I RIA assay, while adequate for identifying severe TC I deficiency, cannot reliably distinguish an overlap between the normal and mildly TC I-deficient states when TC I values sometimes fall within a borderline low-normal range. The problem of phenotypic overlap between normal patients and affected patients with mild, heterozygous disorders is a common one, and not limited to TC I deficiency. Finally, biochemical diagnosis of mild TC I deficiency can also be unreliable if serum is used instead of plasma because cellular TC I can leak readily into serum from cells (Carmel, 2003).

Thus, there is a clinical need for a reliable method for differentiating individuals at risk of or having true cobalamin deficiency from those who have TC I deficiency. This need may be substantial and widespread. Approximately 4% of the population have low serum cobalamin levels (Pfeiffer et al, 2007), and many of these low cobalamin levels are unexplained (Carmel, 2000). A prospective survey found that 15% of unexplained low cobalamin levels are associated with TC I deficiency rather than cobalamin deficiency (Carmel, 2003). If so, low cobalamin levels caused by TC I deficiency may be as common or more common than low cobalamin levels caused by pernicious anemia, and may occur in approximately 1 million or more Americans. Although TC I deficiency can sometimes be an acquired condition, known acquired causes are rare and are usually associated with low granulocyte counts and thus easily identified (Carmel, 1981; Carmel, 1983). Heritable TC I deficiency appears to be more common, but careful surveys and studies are needed.

### SUMMARY OF THE INVENTION

The present invention relates to the discovery that mutations in the TCN1 gene result in a transcobalamin I (TC I) deficiency. Accordingly, the present invention provides for a method of diagnosing a TC I deficiency comprising detecting at least one TCN1 gene mutation or at least one TC I isoform or TC I protein fragment expressed by a mutant TCN1 gene.

In a non-limiting embodiment of the invention, the TCN1 mutation detected is a deletion of nucleotide 270 of the human TCN1 gene.

In another non-limiting embodiment of the invention, the TCN1 mutation detected is a C→T nonsense point mutation at nucleotide 315 of the human TCN1 gene.

In another non-limiting embodiment of the invention, the TCN1 mutation detected is a G→T missense point mutation at nucleotide 999 of the human TCN1 gene.

In another non-limiting embodiment of the invention, the TCN1 mutation detected is a T→ C missense point mutation at nucleotide 475 of the human TCN1 gene.

In one embodiment, the TCN1 mutation is a deletion at nucleotide 270 of a human TCN1 gene. In a further embodiment, the mutation is a deletion of nucleotide 270 of the human TCN1 gene, and the mutation is present at a higher frequency in non-Caucasian or black individuals (e.g., individuals of non-European ancestry) than in Caucasian or white individuals (e.g., individuals of European ancestry).

In other embodiments, the TCN1 mutation is a C→T nonsense point mutation at nucleotide 315 of a human TCN1 gene. In a further embodiment, the mutation is a C→T nonsense point mutation at nucleotide 315 of the human TCN1 gene, and the mutation is present at a higher frequency in non-Caucasian or black individuals (e.g., individuals of non-European ancestry) than in Caucasian or white individuals (e.g., individuals of European ancestry).

In other embodiments, the TCN1 mutation is a G→T missense point mutation at nucleotide 999 of a human TCN1 gene. In a further embodiment, the mutation is a G→T missense point mutation at nucleotide 999 of the human TCN1 gene, and the mutation is present at a higher frequency in Caucasian or white individuals (e.g., individuals of European ancestry) than in non-Caucasian or black individuals (e.g., individuals of non-European ancestry).

In one non-limiting embodiment, the TCN1 gene mutation is associated with a second disorder, for example, sickle cell anemia and sickle cell trait.

In yet another non-limiting embodiment of the invention, the TC I protein isoform fragment detected is a 70 amino acid polypeptide defined by SEQ ID NO:5.

In other non-limiting embodiment of the invention, the TC I protein isoform fragment detected is a 72 amino acid polypeptide defined by SEQ ID NO:7.

In yet another non-limiting embodiment of the invention, the TC I protein isoform detected is an amino acid polypeptide defined by SEQ ID NO:27, wherein SEQ ID NO:27 is a mutated human TC I polypeptide comprising a substitution of the amino acid tyrosine for the amino acid aspartic acid at position 301 of the TC I polypeptide.

In yet another non-limiting embodiment of the invention, the TC I protein isoform detected is an amino acid polypeptide defined by SEQ ID NO:29, wherein SEQ ID NO:29 is a mutated human TC I polypeptide comprising a substitution of the amino acid serine for the amino acid phenylalanine at position 126 of the TC I polypeptide.

As noted above, detection of these mutations indicates that a TC I deficiency exists. In one non-limiting embodiment, detection of a mutation in one allele of a subject is indicative of a mild TC I deficiency. In another non-limiting embodiment, detection of a mutation in both alleles of a subject is indicative of a severe TC I deficiency.

In another embodiment, the TC I deficiency is diagnosed in a sample from a mammal, preferably a human, wherein the sample, whether RNA, DNA, for example, genomic DNA, or TC I protein, is, for example, plasma, serum, cerebrospinal fluid, sputum, saliva, breast milk, tear, bile, semen, vaginal secretion, amniotic fluid, urine, stool, leukocytes, bone marrow cells, buccal cells, fibroblasts, and tissue biopsies sample.

According to the invention, the TCN1 mutation can be detected in the genomic DNA of a mammal, preferably a human, through the use of, for example, the polymerase chain reaction (PCR), quantitative PCR, microarrays, and nucleic acid sequencing.

In a further embodiment, genomic DNA can be derived from tissues, cells (e.g. leukocytes, bone marrow cells, skin cells, fibroblasts, buccal cells, and tissue biopsies) and/or cells in biological fluids from a mammal or human to be tested.

In other embodiments, the TCN1 mutation can be detected by immunodetection of a mutant TC I isoform, wherein the immunodetection assay can be, for example, an ELISA, Western blot, or radioimmunoassay (RIA).

The present invention also provides for isolated monoclonal and polyclonal antibodies specific for a TC I isoform, or a region of a TC I isoform, for example, the TC I isoforms defined by SEQ ID NO:5, SEQ ID NO:7, SEQ ID NO: 27 or SEQ ID NO: 29.

In one embodiment, the monoclonal antibody is a human antibody.

In other embodiments, the monoclonal antibody is an antibody to human TC1 isoform protein.

In other embodiments, the isolated antibody binds to either the D301 wild type peptide or isoform or the mutated Y301 peptide or isoform of TC I defined by SEQ ID NO:27.

In other embodiments, the isolated antibody binds to either the D301 wild type peptide or isoform or the mutated S126 peptide or isoform of TC I defined by SEQ ID NO:29.

In another embodiment, the present invention provides for a kit for detecting at least one TCN1 mutation, wherein the kit comprises a plurality of oligonucleotide primers, each of which is capable of specifically hybridizing to a desired TCN1 exon or region.

The present invention also provides for tools and methods of studying the in vitro and in vivo behavior and properties of a TC I polypeptide, for example, TC I protein folding and TC I protein folding stability.

In one embodiment, a normal TCN1 gene can be replaced by a mutant TCN1 gene by homologous recombination. Replacing the normal gene with the mutated TCN1 gene enables examination of TC I protein behavior, for example, protein folding, trafficking and clearance, in vivo.

In one embodiment, a TC I isoform expressed from a mutant TCN1 can be used to study the behavior and properties of a mutant TC I that leads to reduced plasma TC I levels and to study binding of cobalamin by the TC I.

In another embodiment, the TC I isoform that is used to study the behavior and properties of a mutant TC I that leads to reduced plasma TC I levels is, for example, the TC I isoforms defined by SEQ ID NO:5, SEQ ID NO:7, SEQ ID NO: 27 or SEQ ID NO: 29.

In other embodiments, the kit for detecting at least one TCN1 mutation comprises at least one antibody specific for a region of a TC I isoform, and a means of detecting the antibody when it is bound to the TC I isoform.

The present invention also provides a method of screening for and differentiating among individuals suspected of having cobalamin deficiency because low serum cobalamin levels were detected. Screening the individual for at least one TCN1 mutation or at least one TC I isoform in a sample from the individual can be done, wherein detecting the TCN1 mutation or detecting a TC I isoform indicates a TC I deficiency and not a true cobalamin deficiency.

The present invention also provides methods for increasing the level of TC I in an individual comprising the step of inhibiting TC I fucosylation in the individual. In a further embodiment, inhibiting TC I fucosylation decreases the rate of TC I clearance from the individual's blood.

In one embodiment, TC I fucosylation is inhibited by reducing the expression of α,1,2-fucosyltransferase (FUT2) in the individual, for example, by using antisense or RNAi technologies to regulate FUT2 expression or by using small molecule inhibitors or neutralizing antibodies against FUT2 protein.

In a further embodiment, inhibiting TC I fucosylation in an individual increases serum cobalamin levels in the individual.

In other embodiments, the present invention provides a method for decreasing the level of TC I in an individual comprising the step of increasing TC I fucosylation in the individual. In a further embodiment, increasing TC I fucosylation increases the rate of TC I clearance from the individual's blood.

In one embodiment, TC I fucosylation is increased by increasing the expression of α,1,2-fucosyltransferase (FUT2) in the individual, for example, by administering a FUT2 protein or an expression vector comprising a FUT2 gene operably linked to a promoter to the individual.

In a further embodiment, increasing TC I fucosylation in an individual decreases serum cobalamin levels in the individual.

### BRIEF DESCRIPTION OF THE DRAWINGS

FIG. 1 Shows the TC I genotypes, TC I levels, and cobalamin levels in members of two families that include severe and mild phenotypes of TC I deficiency and a lone member of a third family exhibiting a mild TC I deficiency phenotype.

FIG. 2 Shows examples of the nucleic acid sequences of the primers used for PCR of human genomic TCN1 DNA.

FIG. 3 Shows the nucleic acid sequence of the human transcobalamin I gene (TCN1) (SEQ ID NO:1).

FIG. 4 Shows the amino acid sequence of the polypeptide encoded by the human transcobalamin I gene (TCN1) (SEQ ID NO:2).

FIG. 5 Shows the nucleic acid sequence of a mutant TCN1 comprising a deletion of nucleotide 270 of human TCN1 (SEQ ID NO: 3).

FIG. 6 Shows the amino acid sequence of the 70 amino acid polypeptide expressed by a mutant human TCN1 comprising a deletion of nucleotide 270 (SEQ ID NO: 5).

FIG. 7 Shows the nucleic acid sequence of a mutant human TCN1 comprising a C→T nonsense point mutation at nucleotide 315 of TCN1 (SEQ ID NO: 6).

FIG. 8 Shows the amino acid sequences of the 72 amino acid polypeptide expressed by a mutant human TCN1 comprising a C→T nonsense point mutation at nucleotide 315 (SEQ ID NO: 7).

FIG. 9 Shows an association of transcobalamin I (TC I) deficiency with sickle cell disease and other β-globin hemoglobinopathies.

FIG. 10 Shows characteristics of severe and mild transcobalamin I (TC I) deficiency compared with those of cobalamin deficiency and also compares them to characteristics of both clinical and subclinical cobalamin deficiency.

FIG. 11 Shows the genotypes at nucleotide position 999 of TCN1 in patients with a TC I deficiency described in Example 3, and the genotypes of their relatives.

FIG. 12 Shows the nucleic acid sequence of a mutant human TCN1 comprising a G→ T missense point mutation at nucleotide 999 of TCN1 (SEQ ID NO: 26).

FIG. 13 Shows the amino acid sequence of the polypeptide expressed by a mutant human TCN1 comprising a G→T missense point mutation at nucleotide 999 (SEQ ID NO: 27).

FIG. 14A-C Shows a three-dimensional structure of human TC II (not TC I, whose structure is undetermined because of its heavy glycosylation, but may be inferred in part from homologies with TC II). The conserved aspartic acid corresponding to Asp 301 of TC I is highlighted in red (indicated by an arrow). Cobalamin is the green and blue stick figure of the tetrapyrrole (indicated by an arrowhead). The structure was constructed using PyMOL (PDB ID: 2BB5, see Wuerges et al., 2006, Proc Natl Acad Sci USA; 103:4386-4391). A illustrates the lack of proximity between amino acid 301 substitution (red amino acid; arrow) on the surface at the bottom of the model and the cobalamin (green stick figure; arrowhead) buried in the cleft between the α and β domains shown just above the center of the model. B shows the same structure as in A, but from a different angle, where the cobalamin (blue and green stick figure; arrowhead) is buried more deeply within the cleft, which is more apparent on the left. C illustrates the perspective and relationships from B with a ribbon model.

FIG. 15 Shows the carbohydrate content in TC I isoforms obtained from different sources. The abnormal isoforms in acquired diseases with pathologically elevated plasma TC I and cobalamin levels are underlined. The normal fucose-to-sialic acid ratio in this illustration is between 1.8 and 5.0, but in the abnormal, disease-related isoforms, the ratios are only 0.5 and 1.3 (see Burger et al, J Biol Chem 1975; 250:7700-6).

FIG. 16 Shows the nucleic acid sequence of a mutant human TCN1 comprising a T→ C missense point mutation at nucleotide 475 of TCN1 (SEQ ID NO: 28).

FIG. 17 Shows the amino acid sequence of the polypeptide expressed by a mutant human TCN1 comprising a T→C missense point mutation at nucleotide 475 (SEQ ID NO: 29).

FIG. 18 Shows a comparison of the relevant clinical and laboratory findings in the two patients with sickle cell disease and a subnormal serum cobalamin level due to either cobalamin deficiency or TC I deficiency described in Example 2.

### DETAILED DESCRIPTION

The present invention is based on the discovery that a mutation in the gene encoding transcobalamin I (TCN1) can result in a transcobalamin I (TC I) deficiency. Detecting a mutation in the gene encoding transcobalamin I (TCN1) and/or TC I isoforms (the altered protein products of a mutated TCN1 form) may be used for detecting and/or diagnosing an individual who has or is at risk of TC I deficiency. These methods and compositions will be useful in the differential diagnostic evaluation of persons with low cobalamin levels and, thus, discriminating between a patient who has TC I deficiency and does not need cobalamin prophylaxis and/or treatment and another patient who does not have TC I deficiency but rather a primary cobalamin deficiency and is in need of treatment. In one embodiment, methods and compositions of the present invention are used to identify such patients for treatment of TC I deficiency.

For clarity and not by way of limitation, this detailed description is divided into the following sub-portions:

### (i) Definitions;

(ii) Transcobalamin I deficiency;
(iii) Vectors for cloning, gene transfer, and expression;
(iv) Antibodies reactive with TC I isoforms;
(v) Assays for the detection of TC I deficiency; and

### (iv) Kits.

### DEFINITIONS

The present invention provides methods, compositions, and kits for determining TC I deficiency. Methods for detecting TCN1 mutations and/or the protein products of a mutant TCN1 are useful for detecting and/or diagnosing those who have or are at risk of TC I deficiency. These methods and compositions will be useful in the differential of diagnosis and correct treatment of persons with low serum levels of cobalamin, which are usually taken to represent cobalamin deficiency but are often caused by TC I deficiency instead. The functions of TC I are currently unclear but may become better understood, in part by application of the molecular TC I techniques described herein to clearly identify new cases of TC I deficiency. If important health consequences that suggest the benefits of treatment become known, for example, through application of the present invention, methods for detecting and diagnosing TC I deficiency will become useful for the treatment of patients with TC I deficiency as well.

The terms “transcobalamin I” or “TC I” as used herein refer to a polypeptide of granulocytic and exocrine gland epithelial cell origin which binds cobalamin. In one non-limiting embodiment, the TC I is a human TC I. Human TC I is encoded by the human TC I gene (TCN1) (GenBank accession number NM_{—}001062) (SEQ ID NO:1), a nucleic acid which encodes the human TC I polypeptide. Alternatively, TC I can be encoded by any nucleic acid molecule exhibiting at least 50%, at least 60%, at least 70%, at least 80%, at least 90%, at least 95%, or up to 100% homology to the TCN1 gene (as determined by standard software, e.g. BLAST or FASTA), and any sequences which hybridize under standard conditions to these sequences.

The sequence of the human TCN1 gene, located on chromosome 11q11-q12.3 (Johnston et al., 1992; Shows et al., 1996), has regions of homology with the genes for TC II and intrinsic factor (Johnston et al., 1989, Johnston et al., 1992; Platica et al., 1991). TCN1 has 9 exons of 59 to 191 basepairs (bp) and 8 introns of 160 by to 3.2 kb, and an alternate transcription start site 60 by upstream of the predominant initiation site which appears to operate in a minority of transcripts (Johnston et al., 1992).

In other non-limiting embodiments, a TC I of the invention may be characterized as having an amino acid sequence depicted in SEQ ID NO:2, (GenBank accession number: NP_{—}001053), or any other amino acid sequence at least 60%, at least 70%, at least 90% or at least 95% homologous thereto.

The TC I may be a recombinant TC I polypeptide encoded by a recombinant nucleic acid, for example, a recombinant DNA molecule, or may be of natural origin.

As used herein, a “TCN1 mutation” or “mutant TCN1” refers to a mutation in the gene encoding TC I. In one non-limiting embodiment, the expression of a TCN1 mutation may result in a TC I deficiency when compared to TC I expression from a wild type TC I gene that does not have the mutation. TCN1 mutations may be associated with low TC I levels in individuals and are thus predictive of TC I deficiency. The TCN1 mutation may be, for example, but not limited to, a point mutation, such as a missense or nonsense mutation, a deletion, insertion, frameshift mutation, duplication, or an inversion.

In other non-limiting embodiments, the TCN1 mutation results in the expression of a TC I isoform that is not functional, for example, a TC I that is unable, or has a reduced ability, to bind cobalamin. Examples of such TC I isoforms include, but are not limited to, truncated TC I polypeptides.

In one non-limiting embodiment, the TC I isoform comprises an amino acid sequence defined by SEQ ID NO:5.

In another non-limiting embodiment, the TC I isoform comprises an amino acid sequence defined by SEQ ID NO:7.

In another non-limiting embodiment, the TC I isoform comprises an amino acid sequence defined by SEQ ID NO:27.

In another non-limiting embodiment, the TC I isoform comprises an amino acid sequence defined by SEQ ID NO:29.

In one preferred non-limiting embodiment, the TCN1 mutation is a deletion of nucleotide 270 (Guanine) of the human TCN1 gene, resulting in a mutant TCN1 nucleic acid sequence (SEQ ID NO:3), wherein nucleotide 270 is in exon 2 of the TCN1 gene, and wherein the nucleotides of the TCN1 gene are numbered according to the TCN1 gene defined by GenBank accession number NM_{—}001062 (SEQ ID NO:1). The deletion of nucleotide 270 causes a translation frame shift after amino acid 57 of the TC I protein. Following the frame shift there is an insertion of 13 amino acids (CCPSNLLESRSKP) (SEQ ID NO:4) not found in the wild-type TC I protein, followed by a stop codon, resulting in a truncated 70 amino acid polypeptide (SEQ ID NO:5).

In other preferred non-limiting embodiments, the TCN1 mutation is a C>T nonsense point mutation at nucleotide 315 of the TCN1 gene, resulting in a mutant TCN1 nucleic acid sequence (SEQ ID NO:6), wherein nucleotide 315 is in exon 2 of the TCN1 gene, and the TCN1 gene is numbered according to the TCN1 gene defined by GenBank accession number NM_{—}001062 (SEQ ID NO:1). The C>T point mutation at nucleotide 315 results in a stop codon following amino acid 72 of TC I. The TC I polypeptide isoform produced by the 315 C>T mutation results in a truncated 72 amino acid polypeptide (SEQ ID NO:7).

In other preferred non-limiting embodiments, the TCN1 mutation is a G>T missense point mutation at nucleotide 999 of the TCN1 gene, resulting in a mutant TCN1 nucleic acid sequence (SEQ ID NO:26), wherein nucleotide 999 is in exon 6 of the TCN1 gene, and the TCN1 gene is numbered according to the TCN1 gene defined by GenBank accession number NM_{—}001062 (SEQ ID NO:1). The G>T point mutation at nucleotide 999 results in an amino acid substitution at the highly conserved amino acid 301 of TC I, wherein the small, acidic amino acid aspartic acid normally present at position 301 of a TC I polypeptide (SEQ ID NO:2) is replaced with the polar, aromatic amino acid tyrosine (i.e., a D301Y mutation). The TC I polypeptide isoform produced by the 999 G>T mutation results in a 433 amino acid polypeptide (SEQ ID NO:27).

In other preferred non-limiting embodiments, the TCN1 mutation is a T>C missense point mutation at nucleotide 475 of the TCN1 gene, resulting in a mutant TCN1 nucleic acid sequence (SEQ ID NO:28), wherein nucleotide 475 is in exon 3 of the TCN1 gene, and the TCN1 gene is numbered according to the TCN1 gene defined by GenBank accession number NM_{—}001062 (SEQ ID NO:1). The T>C point mutation at nucleotide 475 results in an amino acid substitution at the conserved amino acid 126 of TC I, wherein the larger, aromatic amino acid phenylalanine normally present at position 126 of a TC I polypeptide (SEQ ID NO:2) is replaced with the small, polar, amino acid serine (i.e., a D301S mutation). The TC I polypeptide isoform produced by the 475 T>C mutation results in a 433 amino acid polypeptide (SEQ ID NO:29).

In one non-limiting embodiment, a “severe TC I deficiency” refers to a TC I deficiency wherein TC I in plasma (for example, rapidly processed, EDTA-anticoagulated plasma) and in glandular secretions such as, for example, saliva, is zero (i.e. below the limits of detection by RIA or assays of cobalamin-binding capacity), or negligible, for example, from about 0-1 pmol/l, about 1-5 pmol/l, about 5-10 pmol/l, about 10-15 pmol/l, or from about 15-20 pmol/l. An individual with a severe TC I deficiency may also have moderately low serum cobalamin levels, for example, from about 75-80 pmol/l, about 80-90 pmol/l, about 90-100 pmol/l, about 100-110 pmol/l, or from about 110-120 pmol/l (as assayed by, for example, commercially available cobalamin assay kits, such as competitive binding assays. See Vogeser, 2003), to severely low, for example, from about 0-10 pmol/l, about 10-20 pmol/l, about 20-30 pmol/l, about 30-40 pmol/l, about 40-50 pmol/l, about 50-60 pmol/l, or from about 60-75 pmol/l (see, e.g., Carmel et al., 1969 and Carmel, 2003).

In another non-limiting embodiment, a “mild TC I deficiency” refers to a mild or moderate depression of TC I in rapidly processed, EDTA-anticoagulated plasma, for example, from about 20-50 pmol/l, about 50-70 pmol/l, about 70-90 pmol/l, about 90-110 pmol/l, about 110-130 pmol/l, about 130-150 pmol/l, or from about 150-165 pmol/l (as detected by RIA), but normal levels in secretions, for example, glandular secretions such as saliva. An individual with a mild TC I deficiency may also have a slightly low serum cobalamin levels, for example, from about 120-130 pmol/l, about 130-140 pmol/l, or from about 140-150 pmol/l, or even low-normal, for example, from about 150-200 pmol/l, about 200-250 pmol/l, or from about 250-300 pmol/l (see Carmel et al., 1969 and Carmel, 2003).

In one non-limiting embodiment, a severe TC I deficiency may occur in an individual when the individual is homozygous for the deletion of nucleotide 270 of TCN1.

In another non-limiting embodiment, a severe TC I deficiency may occur in an individual when the individual is homozygous for the C→T nonsense point mutation at nucleotide 315 of human TCN1.

In another non-limiting embodiment, a severe TC I deficiency may occur in an individual when the individual is homozygous for the T→C missense point mutation at nucleotide 475 of human TCN1.

In another non-limiting embodiment, a severe TC I deficiency may occur in an individual when the individual is homozygous for the G→T missense point mutation at nucleotide 999 of human TCN1. In one non-limiting embodiment, an individual who is homozygous for the G→ T missense point mutation at nucleotide 999 of human TCN1 has a severe TC I deficiency wherein TC I levels, for example, serum TC I levels, are undetectable. In other non-limiting embodiments, an individual who is homozygous for the G→T missense point mutation at nucleotide 999 of human TCN1 has a severe TC I deficiency wherein TC I levels, for example, serum TC I levels, are very low but still detectable.

In other non-limiting embodiments, a severe TC I deficiency occurs when an individual is compound heterozygous for the deletion of nucleotide 270 of TCN1 and the C→T nonsense point mutation at nucleotide 315 of human TCN1 (i.e. the individual is heterozygous for both mutations).

In other non-limiting embodiments, a severe TC I deficiency may occur when an individual is compound heterozygous for the deletion of nucleotide 270 of TCN1 and the G→T missense point mutation at nucleotide 999 of human TCN1 (i.e. the individual is heterozygous for both mutations). In one non-limiting embodiment, an individual who is compound heterozygous for the deletion of nucleotide 270 of TCN1 and the G→T missense point mutation at nucleotide 999 of human TCN1 has a severe TC I deficiency wherein TC I levels, for example, serum TC I levels, are undetectable. In other non-limiting embodiments, an individual who is compound heterozygous for the deletion of nucleotide 270 of TCN1 and the G→T missense point mutation at nucleotide 999 of human TCN1 has a severe TC I deficiency wherein TC I levels, for example, serum TC I levels, are detectable.

In other non-limiting embodiments, a severe TC I deficiency may occur when an individual is compound heterozygous for the C→T nonsense point mutation at nucleotide 315 of human TCN1 and the G→T missense point mutation at nucleotide 999 of human TCN1 (i.e. the individual is heterozygous for both mutations). In one non-limiting embodiment, an individual is compound heterozygous for the C→T nonsense point mutation at nucleotide 315 of human TCN1 and the G→T missense point mutation at nucleotide 999 of human TCN1 has a severe TC I deficiency wherein TC I levels, for example, serum TC I levels, are undetectable. In other non-limiting embodiments, an individual is compound heterozygous for the C→T nonsense point mutation at nucleotide 315 of human TCN1 and the G→T missense point mutation at nucleotide 999 of human TCN1 has a severe TC I deficiency wherein TC I levels, for example, serum TC I levels, are detectable.

In other non-limiting embodiments, a severe TC I deficiency may occur when an individual is compound heterozygous for the deletion of nucleotide 270 of TCN1 and the T→C missense point mutation at nucleotide 475 of human TCN1 (i.e. the individual is heterozygous for both mutations). In one non-limiting embodiment, an individual who is compound heterozygous for the deletion of nucleotide 270 of TCN1 and the T→C missense point mutation at nucleotide 475 of human TCN1 has a severe TC I deficiency wherein TC I levels, for example, serum TC I levels, are undetectable. In other non-limiting embodiments, an individual who is compound heterozygous for the deletion of nucleotide 270 of TCN1 and the T→C missense point mutation at nucleotide 475 of human TCN1 has a severe TC I deficiency wherein TC I levels, for example, serum TC I levels, are detectable.

In other non-limiting embodiments, a severe TC I deficiency may occur when an individual is compound heterozygous for the C→T nonsense point mutation at nucleotide 315 of human TCN1 and the T→C missense point mutation at nucleotide 475 of human TCN1 (i.e. the individual is heterozygous for both mutations). In one non-limiting embodiment, an individual who is compound heterozygous for the C→T nonsense point mutation at nucleotide 315 of human TCN1 and the T→C missense point mutation at nucleotide 475 of human TCN1 has a severe TC I deficiency wherein TC I levels, for example, serum TC I levels, are undetectable. In other non-limiting embodiments, an individual who is compound heterozygous for the C→T nonsense point mutation at nucleotide 315 of human TCN1 and the T→C missense point mutation at nucleotide 475 of human TCN1 has a severe TC I deficiency wherein TC I levels, for example, serum TC I levels, are detectable.

In other non-limiting embodiments, a severe TC I deficiency may occur when an individual is compound heterozygous for the G→T missense point mutation at nucleotide 999 of human TCN and the T→C missense point mutation at nucleotide 475 of human TCN1 (i.e. the individual is heterozygous for both mutations). In one non-limiting embodiment, an individual who is compound heterozygous for the G→T missense point mutation at nucleotide 999 of human TCN and the T→C missense point mutation at nucleotide 475 of human TCN1 has a severe TC I deficiency wherein TC I levels, for example, serum TC I levels, are undetectable. In other non-limiting embodiments, an individual who is compound heterozygous for the G→T missense point mutation at nucleotide 999 of human TCN and the T→C missense point mutation at nucleotide 475 of human TCN1 has a severe TC I deficiency wherein TC I levels, for example, serum TC I levels, are detectable.

In another non-limiting embodiment, a mild TC I deficiency may occur in an individual when the individual is heterozygous for a TCN1 mutation selected from the group consisting of a deletion of nucleotide 270 of TCN1, a C→T nonsense point mutation at nucleotide 315 of TCN1, a G→T missense point mutation at nucleotide 999 of TCN1, and a G→T missense point mutation at nucleotide 999 of human TCN1. In a further, non-limiting embodiment, when the individual is heterozygous for one of the three mutations, the individual's genotype does not include the other two mutations

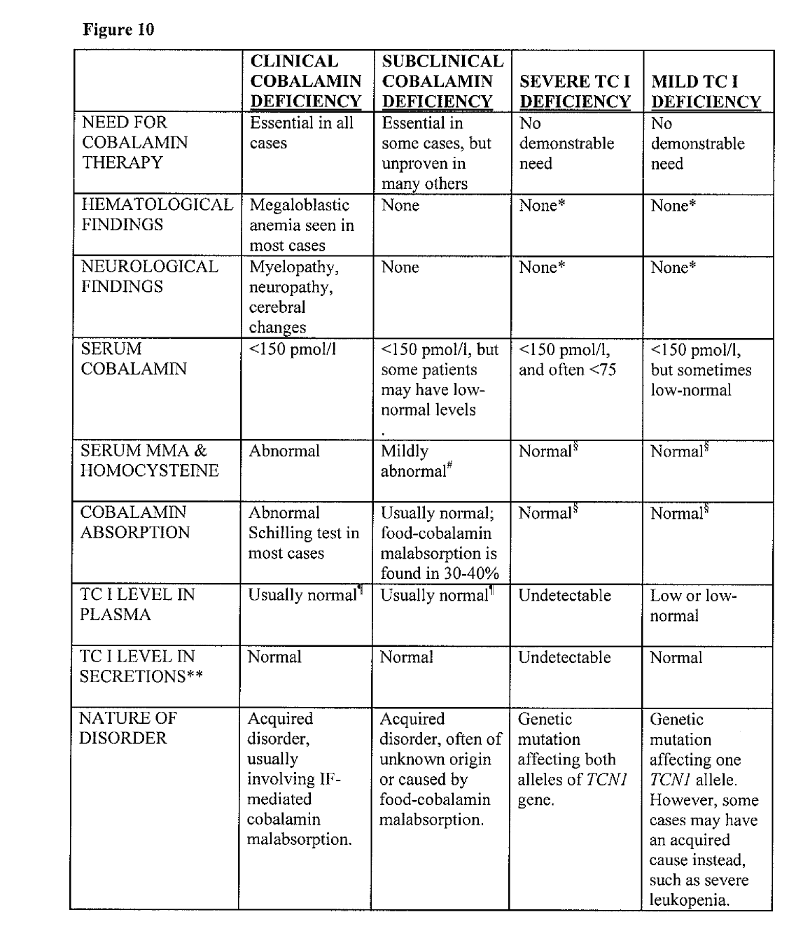

In another non-limiting embodiment of the invention, the terms “true cobalamin deficiency,” or “primary cobalamin deficiency” or “cobalamin deficiency” as used herein refer to a reduced level of serum cobalamin levels in a subject who does not have a TC I deficiency to explain the subject's low cobalamin level. Such subjects usually have cobalamin deficiency at the cellular level that can be documented metabolically, for example, by elevation of serum methylmalonic acid level (Carmel, 2000). Patients with true cobalamin deficiency may also display a characteristic anemia or neurologic symptoms, or, more often than not, may display no clinical findings at all, for example because they have subclinical cobalamin deficiency in which clinical signs have not appeared yet (see FIG. 10). In one non-limiting embodiment, patients with a true cobalamin deficiency exhibit a range of low serum cobalamin levels that is completely indistinguishable from those seen in individuals with either severe or mild TC I deficiency, which may include, for example, low to normal cobalamin levels.

A patient with a true cobalamin deficiency is a candidate for cobalamin therapy, wherein cobalamin may be administered to the patient according to treatment methods known by those skilled in the art. For example, the patient may be administered cobalamin by injection or in the form of an oral pill, wherein the treatment may be lifelong. The patient with true cobalamin deficiency may also require expensive and extensive testing to determine the cause of the true cobalamin deficiency, which often results from malabsorptive gastrointestinal disease. A patient diagnosed with a TC I deficiency according to the methods of the present invention may be spared testing for cobalamin malabsorption and other diseases. In certain cases, treatment may consist, in certain circumstances, solely of regular monitoring by a physician.

The term “cDNA” refers to DNA prepared using messenger RNA (mRNA) as template. The advantage of using a cDNA, as opposed to genomic DNA, is that the cDNA primarily contains coding sequences of the corresponding protein. There may be times when the full or partial genomic sequence is preferred.

The present invention also encompasses DNA segments that are complementary, or essentially complementary, to the sequences described herein. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to a specified nucleic acid segment, under relatively stringent conditions such as those described herein below. Such sequences may encode the entire TC I protein product encompassed herein or functional or non-functional fragments thereof.

A nucleic acid may be contained in a host cell, in some cases, capable of expressing the product of that nucleic acid. In addition to diagnostic and therapeutic considerations, cells expressing nucleic acids of the present invention may prove useful in the context of screening for agents that induce, repress, inhibit, augment, interfere with, block, abrogate, stimulate or enhance the expression, distribution, turnover, or detectability of TCN1 transcripts, TC I polypeptides, or desired fragments of TCN1 transcripts or TC I polypeptides.

Hybridizing segments may be relatively short nucleic acids, often termed oligonucleotides. Sequences of at least 10 bases long, for example, sequences of at least 17 or at least 22 bases long, should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of any number from 8 to 100 or more base pairs will be used, although others are contemplated. Longer polynucleotides are contemplated as well. Such oligonucleotides will find use, for example, as probes in Southern and Northern blots and as primers in amplification reactions.

Suitable hybridization conditions will be well known to those of skill in the art. Accordingly, the nucleotide sequences of the invention may be used for their ability to selectively form duplex molecules with complementary stretches of DNA fragments. Depending on the application envisioned, one will desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by about 0.02 M to about 0.15 M NaCl at temperatures of about 50° C. to about 70° C. Such selective conditions tolerate little, if any, mismatch between the probe and the template or target strand, and would be particularly suitable for isolating protein-encoding DNA segments. Detection of DNA segments via hybridization is well-known to those of skill in the art, and the teachings of U.S. Pat. Nos. 4,965,188 and 5,176,995 (each specifically incorporated herein by reference) are exemplary of the methods of hybridization analyses.

In certain applications, for example, substitution of amino acids by site-directed mutagenesis, it is appreciated that lower stringency conditions are required. Under these conditions, hybridization may occur even though the sequences of probe and target strand are not perfectly complementary, with mismatches at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature.

In certain embodiments, it will be advantageous to employ nucleic acid sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of giving a detectable signal. In preferred embodiments, one will likely desire to employ a fluorescent label or an enzyme tag, such as urease, alkaline phosphatase, luciferase, or peroxidase, instead of radioactive or other environmental undesirable reagents. In the case of enzyme tags, colorimetric indicator substrates are known that can be employed to provide a means visible to the human eye or spectrophotometrically, to identify specific hybridization with complementary nucleic acid-containing samples.

In general, it is envisioned that the hybridization probes described herein will be useful both as reagents in solution hybridization as well as in embodiments employing a solid phase. In embodiments involving a solid phase, the test DNA (or RNA) is adsorbed or otherwise affixed to a selected matrix or surface. This fixed, single-stranded nucleic acid is then subjected to specific hybridization with selected probes under desired conditions. The selected conditions will depend on the particular circumstances based on the particular criteria required (depending, for example, on the G+C content, type of target nucleic acid, source of nucleic acid, size of hybridization probe, etc.). Following washing of the hybridized surface so as to remove nonspecifically bound probe molecules, specific hybridization is detected, or even quantitated, by means of the label.

Probes and primers of the present invention are useful for PCR, qPCR, nucleic acid sequencing, microarray analysis, site-directed, and site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

### TRANSCOBALAMIN I DEFICIENCY

The apparently asymptomatic phenotype associated with TC I deficiency accounts for an underrecognition of the condition, which is usually diagnosed only in patients who are found to have low cobalamin levels, often first detected in older patients because testing for possible cobalamin deficiency is most often attempted in elderly patients. Moreover, the spuriously low cobalamin level of TC I deficiency, although it causes neither metabolic nor clinical cobalamin deficiency, is frequently misdiagnosed as cobalamin deficiency (Carmel, 1969; Carmel, 1983; and Carmel 2003). A prospective survey suggested that despite the limited number of individual case reports in the existing medical literature, TC I deficiency is relatively common. About fifteen percent of low cobalamin levels are accompanied, and may be explained by, the mild form of TC I deficiency (Carmel, 2003). Projections based on this 15% frequency and the likely existence of approximately 10 million Americans with low cobalamin levels (Pfeiffer et al, 2007) suggest that 1-2 million may have TC I deficiency. If so, TC I deficiency may be a more common cause of low cobalamin levels than, for example, pernicious anemia, which is known as the classic and most common cause of clinically relevant cobalamin deficiency. Acquired TC I deficiency also exists, but this condition is usually associated with impaired granulopoiesis, such as aplastic anemia or certain neutropenias, which are infrequent and usually easily recognized conditions. This association occurs because granulocytes are the major source of plasma TC I (Carmel, 1983).

For the reasons stated, in one non-limiting embodiment, the most immediate clinical utility of diagnosing TC I deficiency will be in differentiating its low cobalamin levels from those of true cobalamin deficiency, which is a common diagnostic issue in medicine.

In another non-limiting embodiment, TC I deficiency testing may be used in genetic surveys related to various disorders, or as marker mutations in population surveys. For example, a disproportionate number of reported cases of severe TC I deficiency have been in persons with black or Mediterranean ancestry (Carmel, 2003). Moreover, TC I deficiency may have a linkage of unknown nature with sickle cell anemia and other hemoglobinopathies involving the beta-globin gene, which, like TCN1, resides on chromosome 11 (see, e.g., the families in Example 1). Without being bound to any theory, it may be that such association reflects an old or still extant selection pressure of unknown nature rather than an association based on linkage dysequilibrium.

According to the invention, a “subject” or “patient” is a human or non-human animal. Although the animal subject is preferably a human, the concepts, compounds and compositions of the invention have application in veterinary medicine as well, e.g., for the treatment of domesticated species, farm animal species, and wild animals or zoological garden animals that have TC I which carries most of the cobalamin in their plasma.

In one non-limiting embodiment, the subject or patient has been diagnosed with, or has been identified as having an increased risk of developing, a cobalamin or TC I deficiency, for example, a patient who has been diagnosed with sickle cell anemia, sickle cell trait, or other hemoglobinopathies involving the beta-globin gene, which may have a linkage of unknown nature with TC I deficiency.

In one non-limiting embodiment, a mutation in a human TCN1 gene is associated with a second disorder, for example, sickle cell anemia. As used herein, the terms “associated with a second disorder” mean that the mutation in TCN1 and a second disorder (i.e., a disorder other than a TC I deficiency), exhibit a degree of linkage, wherein the TCN1 mutation and the second disorder are present together in an individual at a higher frequency than if their occurrences were independent of each other.

In another non-limiting embodiment, a subject that has a TCN1 mutation, and a TC I deficiency, may be a candidate for TC I therapy, wherein TC I is administered to increase the subject's level of TC I. TC I may be administered, for example, systemically (e.g. by intravenous injection, oral administration, inhalation, etc.), by intra-arterial, intramuscular, intradermal, transdermal, subcutaneous, oral, intraperitoneal, intraventricular, or intrathecal administration, or may be administered by any other means known in the art.

In one non-limiting embodiment, a subject with a TCN1 mutation may be administered TC I therapy to bind nonfunctional cobalamin analogues, and/or functional cobalamin, and thus prevent access of the analogues and/or functional cobalamin to cells.

In other non-limiting embodiments, a subject with a TCN1 mutation may be administered TC I to bind the subject's endogenous cobalamin and thereby render it unavailable, for example, to microbes present in the subject or infecting the subject.

In other non-limiting embodiments, a subject with a TCN1 mutation may be administered TC I therapy, wherein the TC I is desialylated TC I, and further wherein the desialylated TC I may increase the delivery of cobalamin to the subject's liver, for example, via nonspecific asialoglycoprotein receptors, leading to biliary excretion of the cobalamin.

In other non-limiting embodiments, a subject with a TCN1 mutation may be administered gene therapy, wherein a functional TCN1 gene is introduced into somatic cells of the subject, for example, as a TCN1 expression construct which encodes a functional transcobalamin I protein. Gene therapy can be accomplished by “ex vivo” methods, in which differentiated or somatic stem cells are removed from the individual's body, followed by the introduction of a normal copy of the TCN1 gene into the explanted cells using, for example, a viral vector as the gene delivery vehicle (see, e.g. International Publication No. WO 93/09222). In addition, in vivo direct gene transfer technologies direct the therapeutic gene in situ using a broad range of viral vectors, liposomes, protein DNA complexes, naked DNA and other approaches in order to achieve a therapeutic outcome.

Fucosyltransferases play major roles in cell-cell interactions in many sites. TC I is relatively heavily fucosylated but the responsible fucosyltransferase is unknown. α,1,2-fucosyltransferase, encoded by the FUT2 gene on chromosome 19.q13.3 (GenBank Accession Nos. NM_{—}000511 for one variant and NM_{—}001097638 for a second), which expresses FUT2 polypeptide (GenBank Accession No. NP_{—}000502), is an enzyme that adds fucose to galactosides at the 2 position and regulates the expression of the H-type Lewis histo-blood group antigens on the surface of epithelial cells and in body fluids, determines the secretion status of the ABO antigens, and is involved with thrombospondin and cell adhesion, including possibly Helicobacter pylori adhesion. FUT2 catalyzes the addition of fucose in α,1,2-linkages to the galactose of type I (Gal-β(1-3)-GlcNAc-R) and type 2 (Gal-β(1-4)-GlcNAc-R) disaccharide to form H type 1 and H type 2 antigens, respectively (Kelly et al., 1995, J. Biol. Chem. 270:4640-4649). When homozygous, the rs492602 [G] polymorphism of FUT2 is associated with significantly increased serum cobalamin levels (mean cobalamin levels of 480-490 pmol/l versus 410 pmol/l in non-homozygotes) (Hazra et al., 2008, Nature Genet. 40:1160-1162) as is the closely linked mutation rs602662 [A] (Tanaka et al., 2009, Am J Hum Genet. 84:477-82). Without being bound to any single particular theory, the change in cobalamin levels linked to FUT2 polymorphisms may be more directly effected by changes in fucosylation of TC I, which is a heavily glycosylated protein. TCI proteins with reduced fucosylation (e.g., 9-10 vs. 20-24 fucose residues/mol cobalamin, with simultaneously increased sialylation), are known to be cleared more slowly from the bloodstream than less sialylated but more fucosylated TC I, and as such, are associated with markedly increased plasma TC I levels (Burger et al., 1975, J Biol. Chem. 250:7700-6 and 7707-13). TC I proteins with a reduction in fucosylation, as in conditions such as chronic myelogenous leukemia and hepatocellular carcinoma, are associated with markedly increased serum cobalamin levels. This provides a new example of genetic influence on cobalamin levels by modifying TC I by a process involving genes other than TCN1.

In one non-limiting embodiment, the α,1,2-fucosyltransferase (FUT2) is a human FUT2. Human FUT2 is encoded by the human FUT2 gene (FUT2) as depicted in GenBank accession numbers NM_{—}000511 and NM_{—}001097638, a nucleic acid which encodes the human FUT2 polypeptide. Alternatively, FUT2 can be encoded by any nucleic acid molecule exhibiting at least 50%, at least 60%, at least 70%, at least 80%, at least 90%, at least 95%, or up to 100% homology to the FUT2 gene (as determined by standard software, e.g. BLAST or PASTA), and any sequences which hybridize under standard conditions to these sequences.

In other non-limiting embodiments, a FUT2 of the invention may be characterized as having an amino acid sequence depicted in GenBank accession number: NP_{—}000502, or any other amino acid sequence at least 60%, at least 70%, at least 90% or at least 95% homologous thereto.

The FUT2 may be a recombinant FUT2 polypeptide encoded by a recombinant nucleic acid, for example, a recombinant DNA molecule, or may be of natural origin.

In one non-limiting embodiment, an individual can have both a TCN1 mutation, for example, a G>T missense point mutation at nucleotide 999 of a TCN1 gene, and a FUT2 mutation, for example, a FUT2 mutation that reduces fucosyltransferase activity, wherein the mutations at least partially inhibit, or functionally neutralize, each other's effect on TC I and/or cobalamin levels in the individual. The TCN1 and/or FUT2 mutations can be heterozygous or homozygous.

In one non-limiting embodiment, a 999G>T TCN1 mutation is not associated with a decrease in serum TC I or serum cobalamin levels, for example, when an individual has both a 999G>T TCN1 mutation and is also homozygous for a FUT2 mutation that reduces fucosyltransferase activity compared with the wild type or the heterozygous FUT2 state. In one non-limiting embodiment, the FUT2 mutation is the rs492602 [G] polymorphism. In other non-limiting embodiments, the FUT2 mutation is the rs602662 [A] polymorphism, or a still unidentified but linked polymorphism.

In other non-limiting embodiments, the individual possessing a 999G>T TCN1 mutation that is not associated with a decrease in serum TC I or serum cobalamin levels is also compound heterozygous for two FUT2 mutations, for example, the rs492602 [G] polymorphism and the rs602662 [A] polymorphism.

In another non-limiting embodiment, the level of TC I protein, for example, plasma, blood, serum and/or secretion levels of TC I protein, may be increased in a subject by inhibiting TC I fucosylation, for example, TC I fucosylation in plasma and blood cells, including leukocytes and bone marrow leukocytic precursors, where TC I is synthesized.

In one non-limiting embodiment, TC I fucosylation may be inhibited in a subject by reducing FUT2 activity, or reducing FUT2 expression levels, for example, by administering a compound or drug that reduces FUT2 expression, such as FUT2 antisense or RNAi technology, to raise blood and secretion levels of TC I. Such methods are useful for modulating the cobalamin levels in the blood and serum of an individual, and further, can be used to overcome the low cobalamin level created by TC I deficiency, if desired.

In other non-limiting embodiments, the level of TC I protein, for example, serum TC I protein levels, may be decreased by increasing TC I fucosylation.

In one non-limiting embodiment, TC I fucosylation may be increased by increasing the level of FUT2 enzyme present in the human or other mammalian subject, for example, by administering a FUT2 polypeptide to the subject, or by administering to the subject an expression vector comprising a FUT2 gene operably linked to a promoter.

### VECTORS FOR CLONING, GENE TRANSFER, AND EXPRESSION

Within certain embodiments, expression vectors may be utilized to produce TC I protein products, including fragments or isoforms of TC I, which can then be purified and, for example, be used to generate antisera or a monoclonal antibody with which further studies may be conducted. Expression requires that appropriate signals be provided in the vectors, and which include various regulatory elements, such as enhancers/promoters from viral and mammalian sources, as appropriate that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

In other non-limiting embodiments, a TC I isoform, for example, but not limited to, a wild type TC I (SEQ ID NO: 2), or a mutant TC I isoform (e.g., a mutant TC I isoform as defined by SEQ ID NO: 27), may be expressed in vitro to study the behavior and properties of a TC I polypeptide, or a mutant TC I isoform, in vitro, for example, TC I protein folding. When expressed in vitro the wild type TC I polypeptide or mutant TC I isoform may be expressed recombinantly by a cell in culture, yeast, bacteria, in vitro, or by any other means.

In other non-limiting embodiments, the TC I polypeptide or mutant TC I isoform may be expressed in vivo to study the behavior and properties, for example, protein folding or TC I kinetics, including clearance, in vivo.

As used herein, the term “expression construct” is meant to include any type of genetic construct containing a nucleic acid coding for a gene product in which part or all of the nucleic acid encoding sequence is capable of being transcribed. The transcript may be translated into a protein, or protein fragment, but it need not be. In certain embodiments, expression includes both transcription of a gene and translation of mRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the transcriptional machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. Typically, the promoter is selected for high level or controlled expression, such as lac inducible promoter for use in E. coli, alcohol oxidase for yeast, CMV IE for various mammalian systems, or the polyhedron promoter for Baculovirus. Other elements include polyadenylation signals, origins of replication, internal ribosome entry sites (IRES) and selectable markers (e.g., neomycin, puromycin, hygromycin, DHFR, GPT, zeocin and histidinol).

Transfer of expression constructs into cells also is contemplated by the present invention. These include calcium phosphate precipitation, DEAE-dextran, electroporation, direct microinjection, DNA-loaded liposomes, and lipofectamine-DNA complexes, cell sonication, gene bombardment using high velocity microprojectiles, and receptor-mediated transfection.

In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells.

In one non-limiting embodiment, the virus is a DNA virus, for example, but not limited to, papovaviruses (simian virus 40, bovine papilloma virus, and polyoma) and adenoviruses.

Retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins, making them attractive candidates for transformation of cells. In another non-limiting embodiment, the virus is a retrovirus.

Other viral vectors may be employed as expression constructs in the present invention. For example, vectors derived from viruses such as vaccinia virus, adeno-associated virus (AAV), and herpes viruses may be employed. (Friedmann, 1989; Ridgeway, 1988; Baichwal and Sugden, 1986; Coupar et al., 1988; Harwich et al., 1990).

In other non-limiting embodiments of the invention, the expression construct (including the TC I product) may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers.

### ANTIBODIES REACTIVE WITH TC I ISOFORMS

Generating Antibodies Reactive with TC I Isoforms Resulting from Mutant TCN1

In another aspect, the present invention contemplates an antibody, or fragments thereof, that is immunoreactive with a TC I polypeptide or TC I isoform of the present invention, or any portion thereof. An antibody can be a polyclonal or a monoclonal antibody composition, both of which are preferred embodiments of the present invention. Such antibodies may also include but are not limited to chimeric, human, humanized, single chain, Fab fragments, and a Fab expression library. Means for preparing and characterizing antibodies are well known in the art and can be readily prepared through use of well-known techniques, such as those exemplified in U.S. Pat. No. 4,196,265, incorporated herein by reference (see also, e.g., Kohler and Milstein, 1975 and 1976; Goding, 1986; and Harlow and Lane, 1988). Specifically, techniques developed for the production of chimeric and humanized antibodies have been described by Neuberger, et al., 1984; Brüggemann et al., 1989; and Takeda et al., 1985. Various techniques have been described for the production of single chain antibodies including those in U.S. Pat. Nos. 5,476,786 and 5,132,405 to Huston; and U.S. Pat. No. 4,946,778 each of which is incorporated herein by reference, and these techniques can be adapted to produce for example, TC I-isoform-specific single chain antibodies. An additional embodiment of the invention may utilize the techniques described for the construction of Fab expression libraries of Huse et al., 1989, to allow rapid and easy identification of monoclonal Fab fragments with the desired specificity for a TC I, TC I isoforms, its derivatives, or analogs.

Briefly, a polyclonal antibody can be prepared by immunizing an animal with an immunogen comprising a peptide or polypeptide of the present invention and collecting antisera from that immunized animal. A wide range of animal species can be used for the production of antisera. Typically an animal used for production of anti-antisera is a non-human animal including rabbits, mice, rats, hamsters, guinea pigs, goats, pigs or horses.

Antibodies, both polyclonal and monoclonal, specific for a TC I isoform antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. For example, polyclonal or monoclonal antibodies may be prepared that are reactive for a particular amino acid or amino acid sequence of interest, for example, a TC I isoform defined by SEQ ID NO:5, SEQ ID NO:7, SEQ ID NO:27, or SEQ ID NO:29, or a portion thereof. Such antibodies may be prepared, for example, by immunizing an animal with an immunogen comprising peptides that incorporate the antigen of interest, for example, an amino acid substitution in the mutant TC I polypeptide sequence. Peptides used as immunogens may, for example, comprise 5 to 20 amino acid fragments of the mutant TC I that span the amino acid position of interest, wherein the substituted amino acid is incorporated at different positions within the fragments.

In one, non-limiting embodiment, the antibody, or fragment thereof, is immunoreactive with and binds to the tyrosine at position 301 of the mutant TC I isoform defined by SEQ ID NO:27.

Polyclonal antisera may be obtained, after allowing time for antibody generation, simply by bleeding the animal and preparing serum samples from the whole blood.

The present invention also provides for monoclonal antibodies (Mabs) which are useful in standard immunochemical procedures, such as ELISA and Western blot methods and in immunohistochemical procedures such as tissue staining, as well as in other procedures which may utilize antibodies specific to TC I-related antigenic epitopes. Additionally, monoclonal antibodies specific to a particular TC I of a different species may be utilized in other useful applications.

Both polyclonal and monoclonal antibodies against TC I may be used in a variety of embodiments. For example, they may be employed in antibody cloning protocols to obtain cDNAs or genes encoding other TC I polypeptides or isoforms. They may also be used in inhibition studies to analyze the effects of TC I related peptides in cells or animals. Anti-TC 1 antibodies will also be useful in immunolocalization studies to analyze the distribution of TC I polypeptides or isoforms during various cellular events, for example, to determine the cellular or tissue-specific distribution of TC I polypeptides or isoforms under different points in the cell cycle. A particularly useful application of such antibodies is in purifying native or recombinant TC I, for example, using an antibody affinity column. The operation of all such immunological techniques will be known to those of ordinary skill in the art in light of the present disclosure.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins can also be used as carriers such as, for example, but not limited to, ovalbumin, mouse serum albumin or rabbit serum albumin. Means for conjugating a polypeptide to a carrier protein are well known in the art and include, but are not limited to, glutaraldehyde, m-maleimidobencoyl-N-hydroxysuccinimide ester, carbodimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

### ASSAYS FOR THE DETECTION OF TC I DEFICIENCY

According to the present invention, certain TCN1 mutations are present in individuals with TC I deficiency. Detection of these abnormal TCN1 mutations can be used to detect TC I deficiency and differentiate low cobalamin levels resulting from TC I deficiency from those arising from true cobalamin deficiency. It also can be useful in genetic surveys and as a marker gene mutation. Detection of TC I deficiency is also helpful to identify any adverse health effects associated therewith, and thus detection can be useful for case finding and possible direct treatment of TC I deficiency. Additionally, some TCN1 mutations result in the production of TC I isoforms that can be used as markers in the detection of TC I deficiency. While the present invention is exemplified in humans, its extension to other species including mammals is contemplated. Assays such as RT-PCR, PCR, qPCR, DNA and RNA sequencing, microarray analysis and any other genome-based analyses known in the art, along with any immunoassays known in the art, may be used to detect a TCN1 mutation in a sample. In addition, such analyses may be qualitative or quantitative.

In humans, the TCN1 mutations or resulting TC I protein isoforms may be detected individually or in combination to provide a diagnostic evaluation of TC I deficiency. Other abnormal TCN1 mutations or resulting TC I isoforms from other species may prove useful, alone or in combination, for similar purposes.

### PCR Detection of TCN1 Mutations

In certain embodiments of the present invention, TCN1 mutations are detected by polymerase chain reaction (PCR) or reverse transcriptase-polymerase chain reaction (RT-PCR).

In one non-limiting embodiment, the TCN1 gene is amplified from genomic DNA of a patient. Each of the nine exons of TCN1 may be amplified through PCR by using at least one set of primers for each exon. Following PCR amplification, the PCR amplification products can be sequenced using standard techniques known in the art, and the sequence can be compared to the wild type TCN1 sequence, SEQ ID NO:1. A TCN1 mutation in an exon of TCN1 will be detectable, for example, as a difference between the normal wild type TCN1 nucleic acid sequence (SEQ ID NO:1) and the nucleic acid sequence of the PCR amplification product.

In other non-limiting embodiments, a TCN1 transcription product, or mRNA, may be amplified using RT-PCR. The amplified nucleic acid may then be sequenced and compared to the non-mutant wild type TCN1 nucleic acid sequence (SEQ ID NO:1) to determine whether amplified nucleic acid comprises a TCN1 mutation.

In one non-limiting embodiment, the oligonucleotide primers used for amplifying a TCN1 gene (e.g., PCR and RT-PCR) are complementary to a wild type TCN1 sequence. In other embodiments, the oligonucleotide primers are complementary to a mutant TCN1 sequence.

In a further non-limiting embodiment, the TCN1 nucleic acid (e.g. genomic DNA or RNA) can be derived from tissues, cells and/or cells in biological fluids from a mammal or human to be tested.

Standard cloning and molecular biology techniques are well known in the art and unless otherwise noted, they can be carried out according to various techniques described by Sambrook et al. (2001) Molecular Cloning: A Laboratory Manual. 3rd Ed. Cold Spring Harbor Laboratory Press: Cold Spring Harbor, N.Y., or according to various techniques described in Ausubel et al. eds. (2005) Current Protocols in Molecular Biology. John Wiley and Sons, Inc.: Hoboken, N.J. Each of these references is hereby incorporated by reference in their entirety.

### Quantitative PCR

According to the present invention, individuals may be screened for the presence of a TCN1 mutation through the use of quantitative polymerase chain reaction (qPCR), or quantitative reverse transcriptase-polymerase chain reaction (qRT-PCR), which utilizes competitive techniques employing an internal homologous control that differs in size from the target, for example, by a small insertion or deletion. Non-competitive and kinetic quantitative PCR or RT-PCR may also be used. Experiments may combine real-time, kinetic PCR or RT-PCR detection together with an internal homologous control that can be simultaneously detected alongside the target sequences. In one non-limiting embodiment, real time quantitative PCR may provide the capability of measuring the level of a mutant TCN1 gene product amplified through PCR. In another non-limiting embodiment, quantitative PCR may require only a nominal amount of a sample to perform such experiments.

Quantitative amplification is based on the monitoring of a signal (e.g., fluorescence of a probe) representing copies of a template in cycles of an amplification (e.g., PCR) reaction. In the initial cycles of the PCR, a very low signal is observed because the quantity of the amplification product formed does not support a measurable signal output from the assay. After the initial cycles, as the amount of formed amplification product increases, the signal intensity increases to a measurable level and reaches a plateau in later cycles when the PCR enters into a non-logarithmic phase. Through a plot of the signal intensity versus the cycle number, the specific cycle at which a measurable signal is obtained from the PCR reaction can be deduced and used to back-calculate the quantity of the target before the start of the PCR. The number of the specific cycles that is determined by this method is typically referred to as the cycle threshold (Ct). Exemplary methods are described in, e.g., Heid, 1996; U.S. Pat. Nos. 6,180,349; 6,033,854; and 5,972,602; Gibson, 1996; DeGraves, 2003; and Deiman, 2002.

In one non-limiting embodiment, a method for detection of amplification products is, for example, the 5′-3′ exonuclease activity during PCR reaction (also referred to as the TaqMan™ assay) (see, e.g., U.S. Pat. Nos. 5,210,015 and 5,487,972; Holland, 1991; Lee, 1993). This assay detects the accumulation of a specific PCR product by hybridization and cleavage of a doubly labeled fluorogenic probe (the “TaqMan™” probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if it hybridizes to the segment being amplified. Cleavage of the probe generates an increase in the fluorescence intensity of the reporter dye.

In another non-limiting embodiment, detection of amplification products may utilize, by way of example, and not by way of limitation, energy transfer according to the “beacon probe” method described by Tyagi and Kramer (1996), which is also the subject of U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the Tyagi and Kramer method, the acceptor moiety is a quencher, wherein the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched. When employed in PCR, the molecular beacon probe, which hybridizes to one of the strands of the PCR product, is in the open conformation and fluorescence is detected, while those that remain unhybridized will not fluoresce (Tyagi and Kramer, 1996). As a result, the amount of fluorescence will increase as the amount of PCR product increases, and thus may be used as a measure of the progress of the PCR. Those of skill in the art will recognize that other methods of quantitative amplification are also available.

Various other techniques for performing quantitative amplification of nucleic acids are also known. For example, some methodologies employ one or more probe oligonucleotides that are structured such that a change in fluorescence is generated when the oligonucleotide(s) is hybridized to a target nucleic acid. For example, one such method involves is a dual fluorophore approach that exploits fluorescence resonance energy transfer (FRET), e.g., LightCycler™ hybridization probes, where two oligo probes anneal to the amplification product. The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: Scorpions™ probes (e.g., Whitcombe et al., 1999, and U.S. Pat. No. 6,326,145), Sunrise™ (or Amplifluor™) probes (e.g., Nazarenko et al., 1997, and U.S. Pat. No. 6,117,635), and probes that form a secondary structure that results in reduced signal without a quencher and that emits increased signal when hybridized to a target (e.g., Lux Probes™).

Quantitation of a specific amplified product at the end of an amplification reaction (i.e., end-point PCR) can be employed to quantify the sequences in the final amplified population that match the sequence of DNA which remained undigested by a restriction enzyme. The end-point PCR analysis may be employed under conditions in which the reaction can be analyzed before the reactant nears depletion for a quantitative comparison. Most typically this is done through a comparison of reaction products following a limited number of cycles. For example, a reaction is allowed to cycle 10 times, 15 times, 20 times or 30 times. The quantities of end point PCR products can be compared to each other and an analysis of sequences from the differential enzyme treatments of the DNA sample can be made.

### Detection of TCN1 Mutations Using Nucleic Acid Microarrays

In one non-limiting embodiment of the invention, nucleic acid microarrays, or gene chip technology, may be used to screen and identify patients who carry a TCN1 mutation (see, e.g., U.S. Pat. No. 7,455,975). As used herein, a “microarray” is an array of distinct polynucleotides, oligonucleotides, polypeptides, peptides, or antibodies affixed to a substrate, such as paper, nylon, or other type of membrane; filter; chip; glass slide; or any other type of suitable support.

In one non-limiting embodiment, the microarray technology involves the positioning of highly condensed and ordered arrays of nucleic acid probes, for example, DNA oligonucleotides, on a substrate, for example, a glass slide or nylon membrane. Each oligonucleotide may comprise a nucleotide sequence that is complementary to a portion of a TCN1 expression product which comprises one or more TCN1 mutation, wherein the oligonucleotide can be placed on a single glass slide or nylon membrane. For example, and not by way of limitation, up to 50,000 DNA fragments, may be placed on a single glass slide and up to 5,000 placed on a nylon membrane. The resulting microarrays can then be used to screen for the presence of a TCN1 mutation transcription product expressed in a sample to be screened.

In another non-limiting embodiment, a nucleic acid microarray may be utilized by preparing labeled nucleic acid from a sample to be screened, and hybridizing such labeled nucleic acid with the array. In addition, labeled nucleic acid of a designated control sequences may be prepared (or in the event that the array is sold as part of a kit, could be supplied to the user). Radioactive, colorimetric, chemiluminescent or fluorescent tags may be used for labeling of nucleic acid sequences from the sample and for the control. Numerous techniques for scanning arrays, detecting fluorescent, chemiluminescent, or colorimetric output, are known in the art and may be used for detecting hybridization of a nucleic acid from a test sample to the microarray. For example, a low-cost, high-throughput fluorescent microarray scanning system (ScanArray®, PerkinElmer Life And Analytical Sciences, Inc., Waltham, Mass., USA), or a colorimetric microarray scanner (ArrayIt® SpotWare™, TeleChem International, Inc., Sunnyvale, Calif., USA) may be used. Numerous protocols for the preparation of labeled nucleic acid sequences are publicly available and may be used.

The present invention contemplates the preparation of one or more specialized microarrays (e.g., oligonucleotide microarrays or cDNA microarrays) comprising one or more polynucleotides encoding one or more TCN1 mutation, or complementary sequences, or fragments thereof. In accordance with this aspect of the invention, the oligonucleotide sequences or cDNA sequences include any of the disclosed TCN1 polynucleotides or fragments or combinations thereof, which are expressed in cells of an individual who has one or more mutation in the TCN1 gene, and are contained on a microarray, e.g., a oligonucleotide microarray or cDNA microarray in association with, or introduced onto, any supporting materials, such as glass slides, nylon membrane filters, glass or polymer beads, or other types of suitable substrate material.

Methods for producing and using DNA microarrays are well known in the art (see, e.g. Rampal, 2001, Schena, 2002, and Schena, 2000). Briefly, to determine gene expression using microarray technology, polynucleotides, e.g., RNA, DNA, or cDNA, are isolated from a biological sample, e.g., cells expressing a mutant TCN1. The isolated nucleic acid is detectably labeled, e.g., by fluorescent, enzyme, or chemiluminescent label, and applied to a microarray, e.g., one or more nucleic acid microarrays provided by this invention which comprises, for example, oligonucleotides complimentary to the labeled cellular derived nucleic acid applied to the microarray. The array is then washed to remove unbound material and visualized by staining or fluorescence, or other means known in the art depending on the type of label utilized.

For the purpose of example, and not limitation, microarrays of the invention may be prepared by amplifying full length TCN1 or mutant TCN1 cDNAs or fragments thereof by PCR and arraying the PCR products from a 96-well microtiter plate onto silyated microscope slides using high-speed robotics. Printed arrays may be incubated in a humid chamber to allow rehydration of the array elements and rinsed, in 0.2% SDS for 1 min, twice in water for 1 min and once for 5 min in sodium borohydride solution. The arrays are submerged in water for 2 min at 95° C., transferred into 0.2% SDS for 1 min, rinsed twice with water, air dried and stored in the dark at 25° C.

### Detection of TCN1 Mutations Using Nucleic Acid Sequencing

In another non-limiting embodiment, an individual may be screened for a TCN1 mutation through sequencing (i.e. determining the nucleotide order of a given DNA or RNA fragment) of a genomic DNA or TCN1 expression product present in a sample taken from the individual. Any sequencing methods known in the art may be used to determine the nucleotide order of the TCN1 DNA or RNA.

For example, and not by way of limitation, chain terminator sequencing (i.e. Sanger sequencing) may be used to sequence the TCN1 expression product, wherein extension of a polynucleotide is initiated at a specific site on the template TCN1 nucleic acid (e.g., DNA) by using a short oligonucleotide “primer” complementary to the template at that region. The classical chain-termination method requires a single-stranded DNA template, a DNA primer, a DNA polymerase, radioactively or fluorescently labeled nucleotides, and modified nucleotides that terminate DNA strand elongation (e.g., di-deoxynucleotides). The DNA sample may be divided into four separate sequencing reactions, containing all four of the standard deoxynucleotides (dATP, dGTP, dCTP and dTTP) and the DNA polymerase. One of the four dideoxynucleotides (ddATP, ddGTP, ddCTP, or ddTTP) are added to each of the four reactions, which are the chain-terminating nucleotides, lacking a 3′-OH group required for the formation of a phosphodiester bond between two nucleotides, thus terminating DNA strand extension and resulting in various DNA fragments of varying length.

Newly synthesized and labeled DNA fragments are heat denatured, and separated by size by, for example, gel electrophoresis, with each of the four reactions run in one of four individual lanes of the gel (lanes A, T, G, C). The DNA bands may be visualized by autoradiography or UV light, and the DNA sequence can be directly read off the X-ray film or gel image.

In one embodiment, the primer is labeled (e.g., a fluorescent or radioactive label). In other embodiments, the chain-terminator nucleotides are labeled, for example, in ‘dye terminator sequencing’. In dye terminator sequencing, complete sequencing may be performed in a single reaction, wherein each of the di-deoxynucleotide chain-terminators (e.g., ddATP, ddGTP, ddCTP, and ddTTP) are labeled with a separate fluorescent dye which fluoresces at a different wavelength. The sequence of the template may be determined by separating the synthesized polynucleotide by size and determining the order of the dye signals exhibited by the reaction products.

In other non-limiting embodiments, sequencing may be performed according to the “pyrosequencing” method as described in, for example, Ronaghi, 1996; Ronaghi, 1998; and Nyrén, 2007. Pyrosequencing is a nucleic acid (e.g., DNA) sequencing technique that relies on detection of pyrophosphate release upon nucleotide incorporation rather than chain termination with dideoxynucleotides. Thus, detection of the nucleotide order of the polynucleotide synthesized in the synthesis reaction may be determined in real time as the polynucleotide is extended.

The sequencing of a nucleic acid sample (i.e. determining the nucleotide order of a given DNA or RNA fragment) is not limited to any one technique. The present invention contemplates the use of any sequencing technique known in the art and, for example, new sequencing techniques arising in the future of the sequencing art.

### Immunologic Detection of TC I Isoforms and TCN1 Mutations

In certain embodiments, the present invention entails the use of antibodies in the immunologic detection of protein isoforms resulting from a mutant TCN1. Various useful immunodetection methods have been described in the scientific literature, such as, e.g., Nakamura et al. (1987; incorporated herein by reference). Immunoassays, in their most simple and direct sense, are binding assays. Certain preferred immunoassays include, but are not limited to, enzyme linked immunosorbent assays (ELISAs), Western blots and radioimmunoassays (RIA). Immunohistochemical detection using tissue sections also is particularly useful. However, it will be readily appreciated that detection is not limited to such techniques. For example, Western blotting, dot blotting, FACS analyses, and the like also may be used in connection with the present invention.

In one non-limiting embodiment, the immunological detection methods of the invention can discriminate between a wild type and a mutant TC I polypeptide. For example, when a sample comprises both a wild type and a mutant TC I polypeptide, the immunological methods of the present invention may only detect the mutant TC I polypeptide present in the sample.

In other non-limiting embodiments, the immunological detection methods of the invention can discriminate between a wild type and a mutant TC I polypeptide, such that, for example, when a sample comprises both a wild type and a mutant TC I polypeptide, the immunological methods of the present invention may only detect the wild type polypeptide present in the sample.

In other non-limiting embodiments, the immunological methods of the invention may detect the total TC I polypeptide level present in a sample, wherein the total TC I polypeptide level is comprised of both wild type and mutant TC I polypeptides.

In general, immunobinding methods include obtaining a sample suspected of containing a protein, peptide or antigen, and contacting the sample with an antibody or protein or peptide in accordance with the present invention, as the case may be, under conditions effective to allow the formation of immunocomplexes. Preferred samples, according to the present invention, include, but are not limited to, fluids, such as plasma, serum, cerebrospinal fluid, sputum, saliva, breast milk, tears, bile, semen, vaginal secretion, amniotic fluid, urine or stool sample, as well extracts of cells such as leukocytes, bone marrow cells, buccal cells, fibroblasts and tissue biopsies.

Contacting a biological sample with the protein, peptide or antibody under conditions effective and for a period of time sufficient to allow the formation of immune complexes (primary immune complexes) generally comprises adding the composition, for example an antibody, to the sample and incubating the mixture for a period of time long enough for the antibodies to form immune complexes with TC I isoforms. After this time, the TC I isoform-antibody mixture will be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each of which are incorporated herein by reference. A secondary binding ligand, such as a second antibody or a biotin/avidin ligand binding arrangement, as is known in the art, may also be used to detect the antibody-TC I immunocomplex.

In one non-limiting embodiment, the primary immune complexes may be detected by means of a second binding ligand that has binding affinity for the TC I isoform or for the TC I isoform-specific first antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody that has binding affinity for the TC I isoform is used to form secondary immune complexes, as described above. The second binding ligand contains an enzyme capable of processing a substrate to a detectable product and, hence, amplifying signal over time. After washing, the secondary immune complexes are contacted with substrate, permitting detection.

### ELISA

As a part of the practice of the present invention, the principles of an enzyme-linked immunoassay (ELISA) may be used (Engvall and Perlmann, 1971; Engvall, 1980; Engvall, 1976; Engvall, 1977; Gripenberg et al., 1978; Makler et al., 1981; Sarngadharan et al., 1984). ELISA allows for substances to be passively adsorbed to solid supports such as plastic to enable facile handling under laboratory conditions. For a comprehensive treatise on ELISA the skilled artisan is referred to “ELISA; Theory and Practice” (Crowther, 1995 incorporated herein by reference). In general, in an ELISA, an antigen (or antibody specific for an antigen) is affixed to a surface, and then a specific antibody (or antigen) is washed over the surface so that it can bind to the antigen (or antibody). The antigen-antibody complex may then be detected, for example, by the conversion of a substrate to a detectable signal by an enzyme linked to the antibody, or through the use of labeled secondary or tertiary antibodies specific for the antigen-antibody complex.

The sensitivity of ELISA methods is dependent on the turnover of the enzyme used and the ease of detection of the product of the enzyme reaction. Enhancement of the sensitivity of these assay systems can be achieved by the use of fluorescent and radioactive substrates for the enzymes. Immunoassays encompassed by the present invention include, but are not limited to, those described in U.S. Pat. No. 4,367,110 (double monoclonal antibody sandwich assay) and U.S. Pat. No. 4,452,901 (Western blot). Each of which is hereby incorporated by reference in its entirety. Other assays include immunoprecipitation of labeled ligands and immunocytochemistry, both in vitro and in vivo.

In one non-limiting embodiment, the invention comprises a “sandwich” ELISA, where anti-TC I isoform antibodies are immobilized onto a selected surface, such as a well in a polystyrene microtiter plate or a dipstick. Then, a test composition suspected of containing TC I isoforms, e.g., a clinical sample, is contacted with the surface. After binding and washing to remove non-specifically bound immunocomplexes, the bound antigen may be detected by a second antibody to the anti-TC I isoform antibodies.

In other non-limiting embodiments, polypeptides from the sample are immobilized onto a surface and then contacted with the anti-TC I isoform antibodies. After binding and washing to remove non-specifically bound immune complexes, the bound antibody is detected. Where the initial antibodies are linked to a detectable label, the primary immune complexes may be detected directly. Alternatively, the immune complexes may be detected using a second antibody that has binding affinity for the first antibody, with the second antibody being linked to a detectable label.

In other non-limiting embodiments, an ELISA in which the TC I isoforms are immobilized utilizes antibody competition for detection. In this ELISA, labeled antibodies are added to the wells, allowed to bind to the TC I isoform, and detected by means of their label. The amount of TC I isoform in a sample is determined by mixing the sample with the labeled antibodies before or during incubation with coated wells. The presence of TC I isoform in the sample acts to reduce the amount of antibody available for binding to the well, and thus reduces the ultimate signal.

### KITS

In further embodiments, the invention provides kits, such as an immunological kit, for use in detecting a mutant TCN1 nucleic acid or TC I isoforms in a biological sample. Such kits will generally comprise one or more oligonucleotides and/or antibodies that have specificity for various TCN1 mutant nucleic acids or TC I isoforms.

In one non-limiting embodiment, a kit for detection of a mutant TCN1 nucleic acid will comprise, in suitable container means, one or more control TCN1 mutant nucleic acid, one or more wild-type TCN1 nucleic acid and one or more oligonucleotide that specifically hybridizes to the TCN1 mutant or wild-type nucleic acids, or region thereof, for example, an exonic region, for use in PCR, RT-PCR, qPCR, qRT-PCR, microarray analysis or nucleic acid sequencing. The kit may also comprise one or more polymerase, reverse transcriptase, and nucleotide bases, wherein the nucleotide bases may be further detectably labeled.

In a further non-limiting embodiment, the oligonucleotide primers are immobilized on a solid surface or support, for example, on a nucleic acid microarray, wherein the position of each oligonucleotide primer bound to the solid surface or support is known and identifiable.

In other non-limiting embodiments, the immunodetection kits will comprise, in suitable container means, one or more control TCN1 mutation or TC I isoform, and one or more antibodies that bind to the TCN1 mutations or TC I isoforms, and antibodies that bind to other antibodies via Fc portions.

In certain embodiments, the TC I isoform, TCN1 mutation or primary anti-TC I isoform, antibody may be provided bound to a solid support, such as a column matrix or well of a microtitre plate. Alternatively, the support may be provided as a separate element of the kit.

The immunodetection reagents of the kit may include detectable labels that are associated with, or linked to, the given antibody or antigen itself. Detectable labels that are associated with or attached to a secondary binding ligand are also contemplated. Such detectable labels include, for example, chemiluminescent or fluorescent molecules (rhodamine, fluorescein, green (³H, ³⁵S, ³²P, ¹⁴C, ¹³¹I) or fluorescent protein, luciferase, Cy3, Cy5, or ROX), radiolabels enzymes (alkaline phosphatase, horseradish peroxidase).

The kits may further comprise suitable standards of predetermined amounts, including both oligonucleotides, antibodies and TC Is. These may be used to prepare a standard curve for a detection assay.

The kits of the invention, regardless of type, will generally comprise one or more containers into which the biological agents are placed and, preferably, suitably aliquoted. The components of the kits may be packaged either in aqueous media or in lyophilized form.

The container means of the kits will generally include at least one vial, test tube, flask, bottle, or even syringe or other container means, into which the antibody or antigen may be placed, and preferably, suitably aliquoted. Where a second or third binding ligand or additional component is provided, the kit will also generally contain a second, third or other additional container into which this ligand or component may be placed.

The kits of the present invention will also typically include a means for containing the nucleic acids, TCN1 expression products, or antibodies and any other reagent containers in close confinement for commercial sale. Such containers may include injection or blow-molded plastic containers into which the desired vials are retained.

In one non-limiting embodiment, the kit comprises at least one pair of oligonucleotide primers selected from the group consisting of the following oligonucleotide primer pairs: SEQ ID NO:8 and 9, SEQ ID NO:10 and 11, SEQ ID NO:12 and 13, SEQ ID NO:14 and 15, SEQ ID NO:16 and 17, SEQ ID NO:18 and 19, SEQ ID NO:20 and 21, SEQ ID NO:22 and 23, and SEQ ID NO:24 and 25. The oligonucleotide pairs may be used for amplification (e.g. PCR or qPCR amplification) of a TCN1 nucleic acid (e.g. genomic DNA) sample from a subject.

### EXAMPLES

### Example 1

### Characterization of Three Families with TC I Deficiency Caused by Either of Two Mutations in Exon 2 of TCN1

### Characterization of Subjects

Two families in which at least one individual had the phenotype of severe TC I deficiency and at least one relative with presumptively obligatory heterozygosity had the milder phenotype. A third unrelated patient was also studied. The unrelated third patient was discovered to have mild TC I deficiency independently of having any relatives with severe deficiency. None of the subjects had clinical or metabolic evidence of true cobalamin deficiency or malabsorption despite having low serum cobalamin levels. However, this does not exclude the infrequent possibilty of independent coexistence of TC I deficiency and cobalamin deficiency together, as has been reported (Zittoun et al, 1988).

The severe, putatively homozygous cases of TC I deficiency feature undetectable or trace levels of TC I in plasma and in glandular secretions such as saliva, along with moderately to severely low serum cobalamin levels, whereas obligate heterozygotes have mild to moderate depression of TC I in plasma but normal levels in secretions and slightly low or low-normal serum cobalamin levels (Carmel and Herbert, 1969 and Carmel, 2003).

### Subjects

Two families, among whom severe TC I deficiency had been documented previously, made themselves available for genetic study.

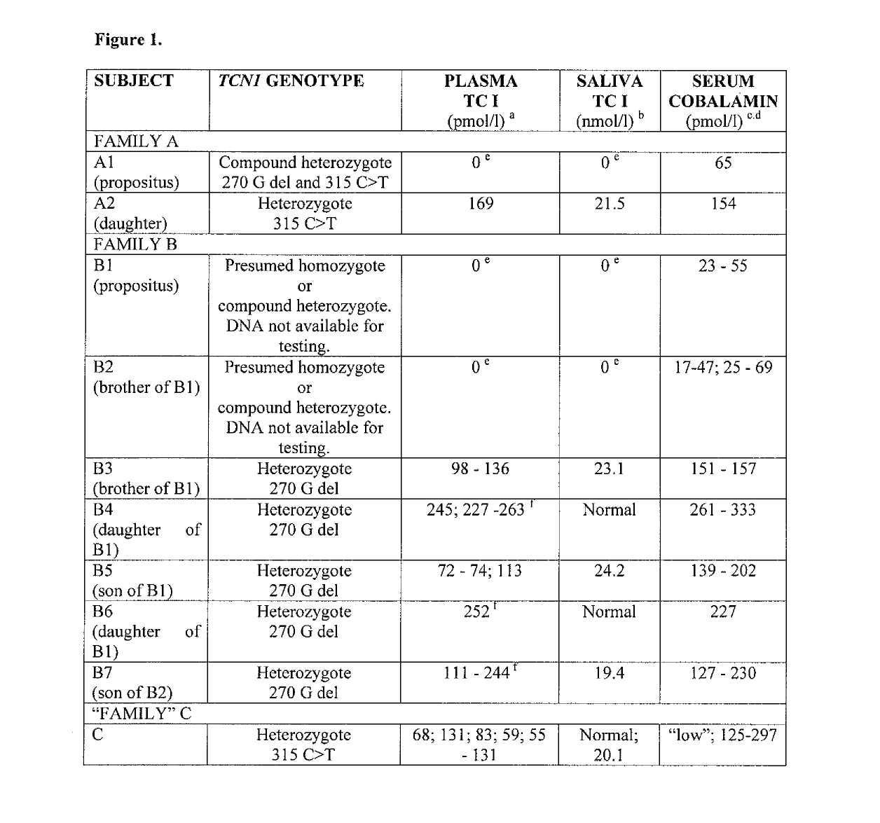

Family A (see FIG. 1): This family was originally described as family A in a survey of low cobalamin levels (Camel, 2003). Patient A1, a 56-year old woman, was found during that survey to have a severe TC I deficiency, and her daughter was then diagnosed with a mild TC I deficiency. Both women had been separately misdiagnosed as having cobalamin deficiency because of low serum cobalamin levels a year or more earlier and received intermittent cobalamin injections without effect. The proposita, patient A1, was of Puerto Rican, Caribbean Indian, and African ancestry and had normal peripheral blood morphology and neurologic findings. She suffered from depression and anxiety, which did not improve with cobalamin therapy. Her other chronic conditions included rheumatic and coronary heart disease, carpal tunnel syndrome, and nonspecific arthritis. Our testing found a serum cobalamin level of 65 pmol/L (normal, 140-750) and total TC I was undetectable in both plasma and saliva by a previously described radioimmunoassay (RIA) with interassay coefficient of variation of 7.2%-13.4% (Carmel, 1983 and Carmel, 2001). She had a normal granulocyte count and normal lactoferrin (Lin, 2001), homocysteine, and methylmalonic acid levels. Like several previous patients with TC I deficiency (Carmel, 1969; Carmel, 1982; and Carmel, 1983), she also had sickle cell trait. An older sister with a low serum cobalamin level refused testing.

Patient A2, the 34-year old only daughter of patient A1, had iron-deficiency anemia, asthma, and keratoconus, with a past history of pseudotumor cerebri attributed to her obesity. A low serum cobalamin level was discovered a year earlier during evaluation for her iron-deficiency anemia, after which she received intermittent cobalamin injections. The blood smear and neurologic examinations were unremarkable. Our testing revealed a borderline low-normal plasma TC I level (169 pmol/L by RIA; normal, 165-454) and normal saliva TC I content, both of which are often seen in obligate heterozygotes for TC I deficiency (Carmel, 2003).

Family B (see FIG. 1): The two severely TC I-deficient members of this previously studied family of Corsican, Puerto Rican, and African ancestry (Carmel, 1969) were deceased (B1 and B2) but their adult relatives, several of whose phenotypes were previously described (Carmel, 1969; Carmel, 1985; Lin, 2001; and Hall, 1977), made themselves available. None had clinical or metabolic signs of cobalamin deficiency. Several additional historical features may be noted in them. Patient B1, the propositus, received long-term cobalamin injections following partial gastrectomy for gastric ulcer at the age of 41 years, had rheumatic heart disease, and died after cardiac surgery; his wife had one spontaneous abortion. B2 developed severe, progressive neurodegenerative disease in his 30s, apparently unrelated to TC I deficiency and unaffected by cobalamin therapy. Patient B3, the brother of B1 and B2, had diabetes mellitus, congestive heart failure, and colon cancer. Of the 3 children of B1, B5 developed Crohn's disease several years after being found to have mild TC I deficiency and took cobalamin supplements intermittently although clinical or metabolic response was not apparent and neither cobalamin malabsorption nor deficiency were demonstrated. B4 and B6 took cobalamin supplements since childhood, when their father's TC I deficiency was diagnosed, and continued it sporadically as adults; B4 had early menopause; B6 had orbital pseudotumor in adolescence, which reversed after steroid therapy, and developed chronic abdominal symptoms of unknown cause after military duty in Iraq in 1991. Patients B1, B5, and B6 have sickle cell trait, and B2 had microcytosis with findings suggestive of alpha-thalassemia. Patients B1 and B2 had absent lactoferrin; their brother, B3, and their two tested children, B5 and B7, had low or low-normal plasma lactoferrin (Lin, 2001). One son of patient B4 has numbness of one arm and leg, blurred vision, and intermittently slurred speech, none of which are compatible with cobalamin deficiency, and is being treated for multiple sclerosis (his father, who is related to family B only by marriage, has a family history of “multiple sclerosis”); he has normal cobalamin (294 pmol/l) and TC I levels (542 pmol/l).

Patient C (see FIG. 1): A 32-year old African-American man with sickle cell anemia had been found to have a low serum cobalamin level and was begun on cobalamin supplements in addition to his long-standing folic acid supplements. The patient had no noteworthy medical history aside from sickle cell crises. He avoided medical contact except when hospitalized for complications of sickle cell disease. During a later admission, testing revealed a low plasma TC I on three occasions (55, 68, and 131 pmol/l) and normal saliva TC I; these findings were characteristic of mild TC I deficiency, including the mild TC I fluctuation. Serum cobalamin was normal (297 pmol/l) when retested by us as he continued his periodic oral cobalamin; serum cobalamin levels can rise well into the normal range after cobalamin therapy in mild TC I deficiency (but rarely do so when TC I deficiency is severe). He was neurologically normal, had no megaloblastic blood changes, and had normal methylmalonic acid, homocysteine, and gastrin levels and negative intrinsic factor antibody. The patient knew of no relatives with low cobalamin levels or cobalamin therapy and had no immediate family to be tested.

### Reagents and Methods

All primers were made by Gene Link (Hawthorne, N.Y.). All sequencing was performed at the Bioresource Center DNA Sequencing Facility, Cornell University (Ithaca N.Y.). The sequences were compared to the TCN1 sequence (NM_{—}001062).

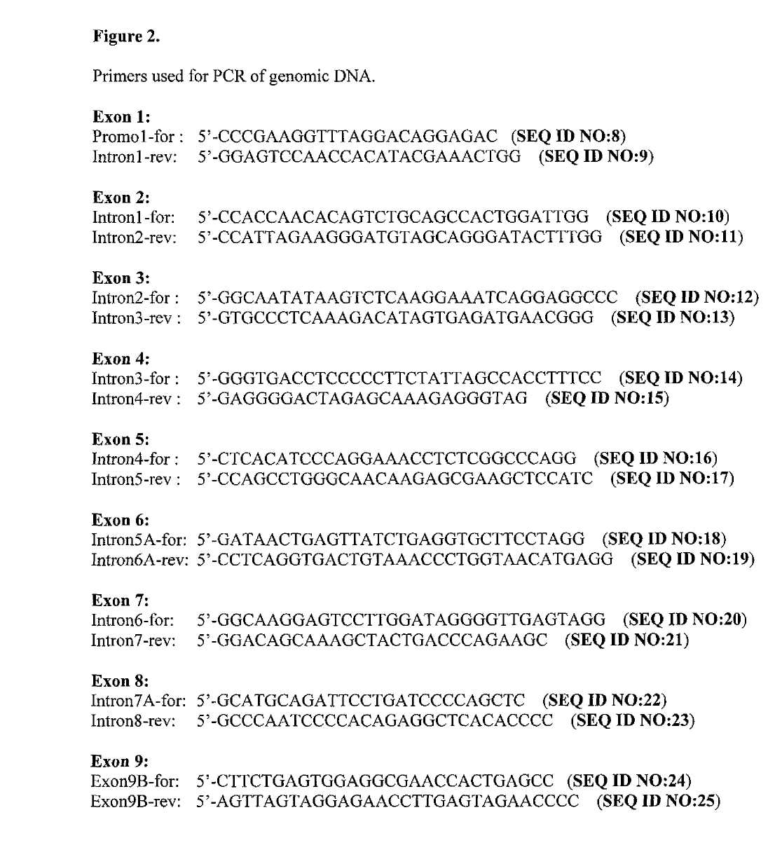

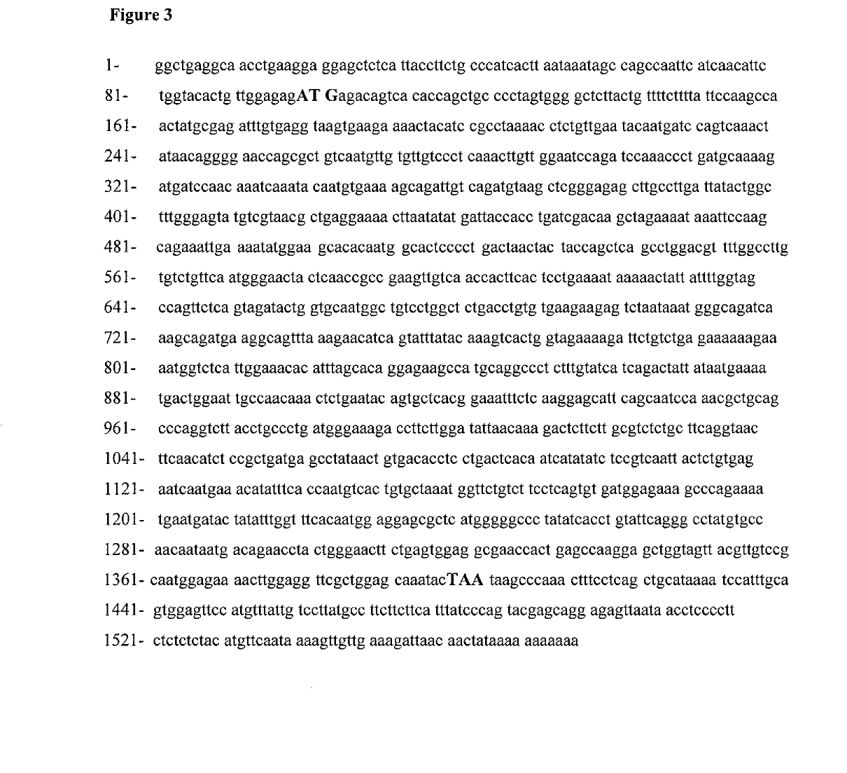

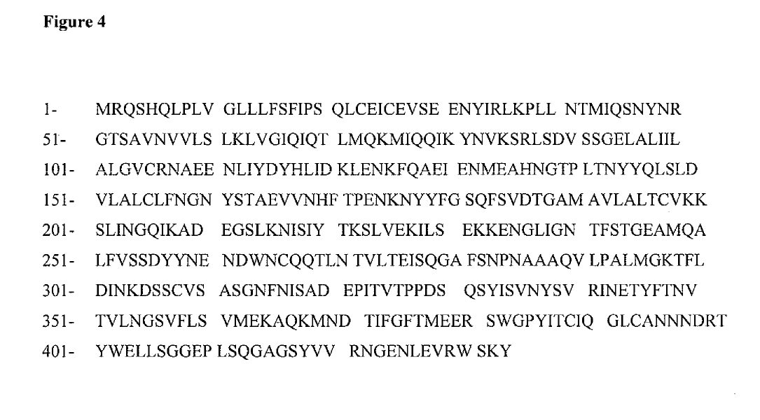

Genomic DNA analysis. To determine whether the TCN1 gene contained mutations in the coding region, a set of 9 primer pairs covering exons 1-9 were designed from intronic sequences encompassing each exon (FIG. 2). These primer sets were used for PCR with genomic DNA, isolated from peripheral blood leukocytes from normal and TC I-deficient patients using QIAamp DNA Blood Mini Kit (Qiagen, Valencia, Calif.). PCR reactions were performed with either Go-Taq (Promega, Madison, Wis.) or Pfu polymerase (Stratagene, La Jolla, Calif.). Following amplification, the products were cloned into either pCR2.1-Topo or pCR4 (Invitrogen, Carlsbad, Calif.) and sequenced.

### Results

### TCN1 Mutations

Nine pairs of primers encompassing the entire TCN1 gene were designed for PCR analysis, covering all 9 exons sequences and thus the entire coding sequence of the gene (FIG. 2).

Family A (see FIG. 1). The genomic DNA of patient A1 was compared with that from subjects with normal TC I levels and the published sequence of the TCN1 gene. The A1 DNA was found to harbor two different mutations in exon 2, one mutation in each allele: the deletion of a G nucleotide at position 270 of the TCN1 cDNA, and the substitution of a T nucleotide for C at position 315. The first mutation causes a translation frame shift after amino acid 57 of the TC I protein. Following the frame shift there is an insertion of 13 amino acids (CCPSNLLESRSKP) (SEQ ID NO:3) not found in the wild-type TC I protein, followed by a stop codon. The second mutation results in a nonsense mutation following amino acid 72.

After establishing that patient A1 is a compound heterozygote for disabling mutations in exon 2, the DNA of her daughter, patient A2, with a milder phenotypic expression, was studied. Her DNA possesses one allele with the C>T substitution at position 315, while the other allele is intact, consistent with her being heterozygous for TC I deficiency.

Family B (see FIG. 1). Only the mildly affected members could be studied, as the two severely TC I-deficient patients were deceased and their DNA was unavailable. The results in B3, the mildly affected brother of severely affected patients B1 and B2, as well as in all four children of patients B1 and B2 (subjects B4-B7), showed the same mutational pattern: DNA from all of them showed one allele with the G deletion at position 270 and one normal allele, thus proving their heterozygous state. No other mutations were found in the other exons.

Patient C (see FIG. 1). Once the results from families A and B became known, patient C was tested as an isolated case of TC I deficiency without any affected relatives. Study of his DNA showed heterozygosity for the C to T mutation in position 315, which had been seen in family A (patient A1). No other mutations were found in exon 2 or in the other exons.

### Discussion

Families A and B both had members with phenotypically severe TC I deficiency and others with phenotypically mild deficiency. In family A, the severely affected, presumably homozygous mother, actually demonstrated compound heterozygosity for two genetic alterations in exon 2 of the 9-exon TCN1 gene. One, the 315C>T mutation, resulted in a nonsense mutation and the other, a G deletion at position 270, caused a frame shift leading to a premature stop codon. Both mutations' premature stop codons assure likely degradation of the transcripts via the nonsense-mediated mRNA decay (NMD) process (Neu-Yilik, 2004 and Conti, 2005). The genomic DNA of her mildly TC I-deficient daughter contained only one affected allele, the 315C>T deletion. In family B, the two severely TC I-deficient brothers, B1 and B2, were deceased and could not be genotyped. However, at least one or both of the alleles in each brother must have had the 270G deletion because all 4 of their mildly affected offspring (subjects B4-B7) were heterozygous for that mutation, as was B3, the mildly affected brother of patients B1 and B2 (FIG. 1); it is unknown if B1 and B2 were homozygous for this 270G deletion or compound heterozygotes with a second mutation.

We also identified an exon 2 mutation in an unrelated patient (patient C) encountered as an isolated case of unexplained mildly low serum cobalamin and TC I levels, rather than as a relative of someone with severe TC I deficiency to point toward him. He turned out to be heterozygous for the 315C>T mutation, one of the mutations found in family A. This observation shows that the two mutations reported here are not private. It also illustrates that even isolated, seemingly nonfamilial cases of mildly low cobalamin and TC I levels should be evaluated for hereditary, often heterozygous TC I deficiency.

These three families (patient C being considered a family of 1 here) demonstrate recurring exon 2 mutations that explain the TC I deficiency and whose gene dose parallels the phenotypic expressions of mild and severe TC I deficiency.

TC I and cobalamin levels. The mutations, when present in homozygous or compound heterozygous doses, seem to abrogate TC I synthesis and produce virtually no functionally (i.e., by binding cobalamin) or immunologically detectable TC I in plasma or secretions, such as saliva. As a result of absent TC I, the major binder of cobalamin in blood, serum cobalamin is decreased because it is carried only by holo-TC II, which is rapidly cleared and maintains normal cobalamin delivery to tissues despite low serum cobalamin (Carmel, 1969), and by minor unidentified proteins (Carmel, 1969; Carmel, 1982; and Hall, 1977). In the milder TC I defect seen in the heterozygotes, plasma TCI is only mildly decreased, and in some unequivocally (and obligatorily) heterozygous cases, such as B4 and B6, TC I can be within the lower part of the reference interval. This may indicate a reference interval in need of recalculation, but phenotypic overlap can occur between heterozygote and control values in recessive disorders (Beaudet, 2001). Indeed, in family B, genotypically identical relatives showed diverse phenotypic expressions; subjects B4 and B6, who were reported to have low TC I when previously tested by RIA elsewhere in 1977 (Hall, 1977), had low-normal TC I and cobalamin levels when retested according to the present application, unlike the low TC I and cobalamin levels in their brother, B4, and their uncle (FIG. 1). Such variations and overlaps can occur for epigenetic and other reasons, including possible individual differences in frequency of mRNA splicing (exon 2 splices, which are frequent in TCN1, often remain in-frame and do not undergo NMD, thus having theoretical potential to lead to higher levels of protein, albeit truncated in size. Variations can also arise from genetic influences other than TCN1 (for example, fucosylation of TC I).

It is noteworthy that each patient's plasma TC I values were consistent on sample reassay but sometimes fluctuated modestly in samples obtained months apart (FIG. 1). Because RIA measures total TC I and recognizes holo-TC I and apo-TC I equally (Carmel, 2003), the temporal fluctuations are unlikely to reflect saturation of TC I by cobalamin supplements.

In general, TC I-deficient persons' cobalamin levels are commensurate with their TC I status, but serum cobalamin levels can rise with cobalamin supplement use, especially in heterozygotes. TC I-deficient patients, depending on extent of TC I deficiency, carry more of their circulating cobalamin on holo-TC II. We observed that the variable extent of holo-TC II accumulation influenced the exact serum cobalamin level. Thus, subjects B4 and B6 had elevated holo-TC II (132 and 175 pmol/l holo-TC II, respectively, assayed with the Axis-Shield immunoassay), which explained their low-normal rather than low total cobalamin levels, compared with the normal holo-TC II level (45 pmol/l) in their brother, B5, whose total cobalamin level was therefore low, as is usual in TC I deficiency.

Phenotypic diagnosis of TC I deficiency. Few laboratories measure plasma TC I, and they often use cobalamin-binding assays that do not distinguish TC I from TC II and detect only the minor apo-TC I fraction of TC I in the blood. Pre-analytic artifacts are frequently overlooked also, such as using serum, which promotes in vitro release of granulocytic TC I (Carmel, 2001), or samples drawn after cobalamin injections have saturated apo-TC I and created spuriously low TC I results. These and other errors probably caused the mistaken report of TC I deficiency (Adcock, 2002) in a patient whose data on our review suggest cobalamin deficiency instead.

Very few research laboratories, and no commercial ones, perform immunoassay of total (apo and holo) TC I, which is currently the most reliable method to diagnose TC I deficiency. Yet, as discussed earlier, TC I RIA can adequately identify severe TC I deficiency, which is uncommon, but RIA may not identify cases of mild TC I deficiency whose TC I levels sometimes overlap with control values in the much more common mildly TC I-deficient states (see family B).

Differentiating TC I deficiency from cobalamin deficiency. The issues just discussed indicate a need for additional tools to diagnose TC I deficiency definitively. The need has considerable clinical relevance because the frequently low cobalamin levels in TC I deficiency mimic cobalamin deficiency: the low cobalamin levels of severe TC I deficiency resemble those of pernicious anemia, and the low to low-normal cobalamin levels in mild TC I deficiency resemble those in mild, subclinical cobalamin deficiency. The need has grown because subclinical cobalamin deficiency now far surpasses severe cobalamin deficiency in frequency (Carmel, 2000). Moreover, normal methylmalonic acid and homocysteine results suggesting that 20-40% of asymptomatic low cobalamin levels in population surveys are spurious (Carmel, 2000) fit with the report that as many as 15% of low cobalamin levels may reflect mild TC I deficiency and 0.6% reflect severe TC I deficiency (Carmel, 2003). Thus, the ability to differentiate TC I deficiency from cobalamin deficiency may have substantial impact on the clinical diagnosis and management of cobalamin deficiency. True cobalamin deficiency can have major clinical complications and requires prompt and often lifelong cobalamin therapy, whereas low cobalamin levels due to TC I deficiency do not. The diagnostic value of identifying TC I deficiency has grown as other causes of cobalamin deficiency, especially cobalamin malabsorption, have become difficult to diagnose (Carmel, 2007).

### REFERENCES

Adcock B. B., and McKnight J. T. Cobalamin pseudodeficiency due to a transcobalamin I deficiency. Southern Med. J., 2002; 95:1060-2.
Ausubel et al. eds. Current Protocols in Molecular Biology. John Wiley and Sons, Inc.: Hoboken, N.J. 2005.
Baichwal, V. R. and Sugden, B. In: Gene Transfer, Kucherlapati R. (Ed.), Plenum Press, New York, pp 117-148, 1986.
Beaudet A L, Scriver C R, Sly W S, Valle D. Genetics, biochemistry, and molecular bases of variant human phenotypes. In: The Metabolic and Molecular Bases of Inherited Disease, 8th ed. (Eds: Scriver C R, Beaudet A L, Sly W S, Valle D) McGraw Hill; New York: 2001. Chap. 1.
Brüggemann, M., Caskey, H. M., Teale, C., Waldmann, H., Williams, G. T., Surani, M. A., and Neuberger, M. S. A repertoire of monoclonal antibodies with human heavy chains from transgenic mice. Proc Natl Acad Sci USA, 1989; 86(17): 6709-6713.
Burger R. L., Mehlman C. S., Allen R. H. Human plasma R-type vitamin B12-binding proteins. I. Isolation and characterization of transcobalamin I, transcobalamin III, and the normal granulocyte vitamin B12-binding protein. J Biol Chem 1975; 250:7700-7706
Burger R L, Schneider R J, Mehlman C S, Allen R H. Human plasma R-type vitamin B12-binding proteins. II. The role of transccobalamin I, transcobalamin III, and the normal granulocyte B12-binding protein in the plasma transport of vitamin B12. J Biol Chem 1975; 250:7707-7713.
Carmel R., and Herbert V. Deficiency of vitamin B₁₂-binding alpha globulin in two brothers. Blood, 1969; 33:1-12.
Carmel R.: Cobalamin-binding proteins in man. In: Contemporary Hematology-Oncology. Silber R, Gordon A S, LoBue I, Muggia F M (eds.), Vol. 2, Plenum, New York, 1981:79-129.
   Carmel R. A new case of deficiency of the R binder for cobalamin, with observations on minor cobalamin-binding proteins in serum and saliva. Blood, 1982; 59:152-156.
Carmel R. An unusual case of autoimmune agranulocytosis with total absence of myeloid precursors: demonstration of diverse sources of R binder for cobalamin in plasma and secretions. Am. J. Clin. Pathol., 1983; 79:611-615.

Carmel R. R binder deficiency: a clinically benign cause of cobalamin pseudo-deficiency. JAMA, 1983; 250:1886-1890.
Carmel R. Current concepts in cobalamin deficiency. Annu Rev Med 2000; 51:357-375.
Carmel R, Brar S, Frouhar Z. Plasma total transcobalamin I. Ethnic/racial patterns and comparison with lactoferrin. Am J Clin Pathol 2001; 116:576-580.
Carmel R. Mild transcobalamin I (haptocorrin) deficiency and low serum cobalamin concentrations. Clin. Chem., 2003; 49:1367-1374.
Carmel R. The disappearance of cobalamin absorption testing: a critical diagnostic loss. J Nutr 2007; 137:2481-2484.
Carmel R. Haptocorrin (transcobalamin I) and cobalamin deficiencies. Clin Chem 2007; 53:367-368.

Conti E, Izaurralde E. Nonsense-mediated mRNA decay: molecular insights and mechanistic variations across species. Curr Opin Cell Biol 2005; 17:316-325.
Coupar R E., Andrew M. E., and Boyle D. B., A general method for the construction of recombinant vaccinia viruses expressing multiple foreign genes. Gene, 1988; 68:1-10.
Cowland J. B., and Borregaard N. The individual regulation of granule protein mRNA levels during neutrophil maturation explains the heterogeneity of neutrophil granules. J. Leukoc. Biol., 1999; 66:989-95.
Crowther, J. R. ELISA: Theory and Practice, Methods in Molecular Biology, 1995; 42:1-218.
DeGraves, et al., High-sensitivity quantitative PCR platform. Biotechniques 2003; 34(1):106-10, 112-5.
Deiman B, et al., Characteristics and applications of nucleic acid sequence-based amplification (NASBA). Mol. Biotechnol. 2002; 20(2):163-79.
Engvall, E. and Perlmann, Enzyme-linked immunosorbent assay (ELISA). Quantitative assay of immunoglobulin G. Immunochem., 1971; 8:871-873.
Engvall, E. Determination of antibodies to D.N.A. by ELISA. Lancet, 1976; 2(8000): 1410.
Engvall, E. Quantitative enzyme immunoassay (ELISA) in microbiology. Med. Biol., 1977; 55(4): 193-200.
Engvall, E. Enzyme immunoassay ELISA and EMIT. Methods Enzymol., 1980; 70(A):419-39.
Faustino N. A., and Cooper T. A. Pre-mRNA splicing and human disease. Genes Devel., 2003; 17:419-37.
Friedmann, T. Progress toward human gene therapy. Science, 1989; 244:1275-1281.
Gibson et al., A novel method for real time quantitative RT-PCR. Genome Research 1996; 6:995-1001.
Goding J. W., In: Monoclonal Antibodies: Principles and Practice, 2d ed., Academic Press, Orlando, Fla., pp 60-61, 71-74, 1986.
Gripenberg M., Linder E., Kurki P., Engvall E. A solid phase enzyme-linked immunosurbent assay (ELISA) for the demonstration of antibodies against denatured, single-stranded DNA in patient sera. Scand J. Immunol., 1978; 7(2):151-7.
Gullberg R. Possible antimicrobial function of the large molecular size vitamin B12-binding protein. Scand J Gastroenterol 1974; 9(suppl 29):19-21.
Hall C A, Begley J A. Congenital deficiency of human R-type binding proteins of cobalamin. Am J Hum Genet.; 29:619-626.
Harlow and Lane, In: Antibodies, a Laboratory Manual, Cold Spring Harbor Laboratory, pp 139-281, 1988.
Hazra A., Kraft P., Selhub J., Giovannucci E. L., Thomas G., Hoover R. N., Chanock S. J., Hunter D. J. Common variants of FUT2 are associated with plasma vitamin B12 levels. Nature Genet. 2008; 40:1160-1162.
Heid et al., Real time quantitative PCR. Genome Methods 1996; 6:986-94.
Heid et al., A novel method for real time quantitative RT-PCR. Genome Methods 1996; 6:995-1001.
Holland et al., Detection of specific polymerase chain reaction product by utilizing the 5′----3′ exonuclease activity of Thermus aquaticus DNA polymerase. Proceedings of the National Academy of Sciences USA 1991; 88:7276-7280.
Horwich A. L., Furtak K., Pugh J., Summers J. Synthesis of hepadnavirus particles that contain replication-defective duck hepatitis B virus genomes in cultured HuH7 cells. J. Virol., 1990; 64:642-650.
   Huse, W. D., Sastry, L., Iverson, S. A., Kang, A. S., Alting-Mees, M., Burton, D. R., Benkovic, S. J., and Lerner, R. A. Generation of a large combinatorial library of the immunoglobulin repertoire in phage lambda. Science, 1989; 246:1275-1281.
Jenks J., Begley J., and Howard L. Cobalamin R binder deficiency in a woman with thalassemia. Nutr. Rev., 1983; 41:277-280.
Johnston, J., Bollekens, J., Allen, R. H., and Berliner, N. Structure and expression of the cDNA encoding transcobalamin I, a neutrophil granule protein. J. Biol. Chem., 1989; 264:15754-15757.
Johnston J., Yang-Feng T., and Berliner N. Genomic structure and mapping of the chromosomal gene for transcobalamin I (TCN1): comparison to human intrinsic factor. Genomics 1992; 12:459-464.
Kelly R. J., Rouquier S., Giorgi D., Lennon G. G., Lowe J. B. Sequence and expression of a candidate for the human Secretor blood group 1,2-fucosyltransferase gene (FUT2). J Biol Chem 1995; 270:4640-4649.
Kelman Z., Prokocimer M., Peller S., Kahn Y., Rechavi G., Manor Y., Cohen A., and Rotter V. Rearrangements in the p53 gene in Philadelphia chromosome positive chronic myelogenous leukemia. Blood, 1989; 74:2318-24.
Kondo H, Kolhouse J F, Allen R H. Presence of cobalamin analogues in animal tissues. Proc Natl Acad Sci USA 1980; 77:817-821.
Kohler, G. and Milstein C., Continuous cultures of fused cells secreting antibody of predefined specificity. Nature, 1975; 256:495-497.
Kohler, G. and Milstein C., Derivation of specific antibody-producing tissue culture and tumor lines by cell fusion. Eur. J. Immunol., 1976; 6:511-519.
Krawczak M., Reiss J., and Cooper D. N. The mutational spectrum of single base-pair substitutions in mRNA splice junctions of human genes: causes and consequences. Hum. Genet., 1992; 90:41-54.
Lee et al., Allelic Discrimination by Nick-Translation PCR with Fluorogenic Probes. Nucleic Acids Res. 1993; 21: 3761-3766.
Lewis B. P., Green R. E., and Brenner S. E. Evidence for the widespread coupling of alternative splicing and nonsense-mediated mRNA decay in humans. Proc. Natl. Acad. Sci. USA, 2003; 100:189-92.
Li, N., Rosenblatt, D. S., and Seetharam, B. Nonsense mutations in human transcobalamin II deficiency. Biochem. Biophys. Res. Comm., 1994; 204:1111-8.
Lin J. C., Borregaard N., Liebman H. A., Carmel R. Deficiency of the specific granule proteins, R binder/transcobalamin I and lactoferrin, in plasma and saliva: a new disorder. Am. Med. Genet., 2001; 100:145-51.
Maniatis, T. and Tasic, B., Alternative pre-mRNA splicing and proteome expansion in metazoans. Nature, 2002; 418: 236-243.
Michael, I. P., Kurlender, L., Mamri, N., Yousef, G. M., Du, D., Grass, L., Stephan, C., Jung, K., and Diamandis, E. P. Intron retention: a common splicing event within the human kallikrein gene family. Clin. Chem., 2005; 51:506-15.
   Morkbak A L, Hvas A M, Lloyd-Wright Z L, Sanders T A B, Bleie O, Refsum H, et al. Effect of vitamin B12 treatment on haptocorrin. Clin Chem 2006; 52:1104-1111.
Nagy E., and Maquat L. E. A rule for termination-codon position within intron-containing genes: when nonsense affects RNA abundance. Trends Biochem. Sci., 1998; 23:198-9.
Nakamura, H. In: Handbook of Experimental Immunology (4th Ed.), Weir, E., Herzenberg, L. A., Blackwell, C., Herzenberg, L. (Eds.), Vol. 1, Chapter 27, Blackwell Scientific Publ., Oxford, 1987.
Namour, F., Helfer, A. C., Quadros, E. V., Alberto, J. M., Bibi, H., Orsing L., Rosenblatt D. S., and Gueant J. L. Transcobalamin deficiency due to activation of an intra exonic cryptic splice site. Br. J. Haematol. 2003; 123:915-20.
Nazarenko et al., A closed tube format for amplification and detection of DNA based on energy transfer, Nuc. Acids Res. 1997; 25:2516-2521.
Neu-Yilik G, Gehring N H, Hentze M W, Kulozik A E. Nonsense-mediated mRNA decay: from vacuum cleaner to Swiss army knife. Genome Biol 2004; 5:218.1-5.
Neuberger, M. S., Williams G. T., and Fox, R. O. Recombinant antibodies possessing novel effector functions. Nature, 1984; 312:604-608.
Nyrén, P. The History of Pyrosequencing. Methods Mol Biology 2007; 373: 1-14.

Pfeiffer C M, Johnson C L, Jain R B, Yetley E A, Picciano M F, Rader J I, Fisher K D, Mulinare J, Osterloh J D. Trends in blood folate and vitamin B-12 concentrations in the United States, 1988-2004. Am J Clin Nutr 2007; 86:718-727.
Platica O., Janeczko R., Quadros E. V., Regec A., Romain R., Rothenberg S. P. The cDNA sequence and the deduced amino acid sequence of human transcobalamin II show homology with rat intrinsic factor and human transcobalamin I. J. Biol. Chem., 1991; 266:7860-3.
Qian L., Quadros E. V., Regec A., Zittoun J., and Rothenberg S P. Congenital transcobalamin II deficiency due to errors in RNA editing. Blood Cells Molec. Dis. 2002; 28:134-42.
Rampal J. B., 2001, DNA Arrays: Methods and Protocols, Humana Press, Inc., Totowa, N.J.
Ridgeway, In: Vectors: A Survey of Molecular Cloning Vectors and Their Uses, Rodriguez R. L. and Denhardt D. T. (Ed.), Butterworth, Stoneham, pp 467-492, 1988.
Ronaghi et al. Real-time DNA sequencing using detection of pyrophosphate release. Analytical Biochemistry 1996; 242(1):84-9.
Ronaghi et al. A sequencing method based on real-time pyrophosphate. Science 1998; 281:363-365.
Sambrook et al. Molecular Cloning: A Laboratory Manual. 3rd ed. Cold Spring Harbor Laboratory Press: Cold Spring Harbor, N.Y. 2001.
Schena M., 2000, Microarray Biochip Technology, Eaton Publishing.
Schena M., 2002, Microarray Analysis, John Wiley & Sons.
Schmucker D., Clemens J. C., Shu H., Worby C. A., Xiao J., Muda M., Dixon J. E., Zipursky S. L. Drosophila Dscam is an axon guidance receptor exhibiting extraordinary molecular diversity. Cell, 2000; 101:671-684.
Shows, T. B., Alders, M., Bennett, S., Burbee, D., Cartwright P, et al. Report of the Fifth International Workshop on Human Chromosome 11 Mapping (1996). Cytogenet Cell Genet., 1996; 74:1-56.
Takeda, S., Naito, T., Hama, K., Noma, T., and Honjo, T., Construction of chimaeric processed immunoglobulin genes containing mouse variable and human constant region sequences. Nature, 1985; 314:452-454.
Tanaka T., Scheet P., Giusti B., Bandinelli S., Piras M. G., et al. Genome-wide association study of vitamin B6, vitamin B12, folic acid, and homocysteine blood concentrations. Am J Hum Genet. 2009; 84:477-482
Tyagi and Kramer, Molecular beacons: probes that fluoresce upon hybridization. Nature Biotech. 1996; 14:303-309.
Vogeser, M. and Lorenzl, S., Comparison of automated assays for the determination of vitamin B12 in serum. Clin Chem. 2007; 40:1342-1345.
Whitcombe et al., Detection of PCR products using self-probing amplicons and fluorescence. Nature Biotechnology 1999; 17:804-807.
Xing Y., Resch A., Lee C. The multiassembly problem: reconstructing multiple transcript isoforms from EST fragment mixtures. Genome Res., 2004; 14:426-41.
Yassin F., Rothenberg S. P., Rao S., Gordon M. M., Alpers D. H., and Quadros E. V. Identification of a 4-base deletion in the gene for inherited intrinsic factor deficiency. Blood, 2004; 103:1515-7.
Zittoun J, Léger J., Marquet J., Carmel R. Combined congenital deficiencies of intrinsic factor and R binder. Blood, 1988; 72:940-943.

### Example 2

### Differentiation of Low Serum Cobalamin Levels with Disparate Origins and Clinical Meanings: Two Instructive Cases of Sickle Cell Disease

Both cobalamin deficiency, such as in pernicious anemia (PA), which is not often considered in patients who have sickle cell disease or in blacks in general, and transcobalamin (TC) I deficiency cause low cobalamin levels. Described herein are two patients with sickle cell disease and low cobalamin levels, one patient whose diagnosis of PA was delayed and one whose mild TC I deficiency was misdiagnosed and treated as PA. The first patient, as the fourth described case, suggests that PA is substantively associated with sickle cell disorders and should be considered whenever anemia worsens unexpectedly, MCV rises, neurological symptoms develop, or crises become more frequent in patients with sickle cell disease. Routine folate supplementation in patients with sickle cell disease, probably superfluous in the era of food fortification, should also be accompanied by annual cobalamin testing. Because low cobalamin levels have very different origins, prognoses, and clinical requirements in PA and TC I deficiency, and because low but clinically equivocal cobalamin levels outnumber clinical cobalamin deficiency, TC I deficiency must be differentiated from PA and other cobalamin deficiency states. TC I deficiency should be considered in any patient whose low cobalamin level is not accompanied by unequivocal clinical or metabolic signs of cobalamin deficiency. However, reliable tools to diagnose TC I deficiency are not widely available. The newly identified heterozygosity for a TCN1 gene mutation, 315C>T, explains the mild TC I deficiency in the second patient, confirms a genetic basis, and may prove diagnostically useful.

### Introduction

Because of the severity of sickle cell anemia, superimposed anemias can be overlooked. One example is PA (defined as cobalamin deficiency caused by the loss of gastric intrinsic factor secretion), which is rarely considered in patients with sickle cell disease. PA is not a common disease and is often regarded as predominantly affecting whites. Its usual onset in old age,^{A} combined with the decreased attainment of old age by patients with sickle cell disease, have made the two anemias seem even less likely to coexist. However, PA may be nearly as common in blacks as in whites,^{B,I} especially in black women, who also tend to develop PA at a much younger age than white women.^{C} The initial reports of coexisting PA and sickle cell disease^{D-F} were considered a rarity, but a fourth patient is described here with this combination, which suggests more than coincidence.

In addition, a patient is reported with sickle cell disease whose low serum cobalamin was initially thought to represent cobalamin deficiency but turned out to reflect transcobalamin (TC) I deficiency instead. The latter provides a very different explanation for a low cobalamin level than patient 1 but may similarly have an increased association with sickle cell disease. The encounter with both these patients in one hospital allows comparisons of clinical approaches to diverse low cobalamin levels in the general population, in blacks more specifically, and in patients with sickle cell disease perhaps most specifically of all.

### Case Descriptions

### Patient 1:

A 24-year old black man with known sickle cell-Hgb C hemoglobinopathy was hospitalized because of a 5-day history of fever, headache, and diffuse joint pains; a longer history of tiredness; and an unexplained 30-lb weight loss over the past year. No specific symptoms or characteristics accompanied his headache, fever, or joint pains. He was previously unknown to our hospital, except for a brief clinic visit 6 weeks earlier, at which his hemoglobin (Hgb) level was 9.8 g/dl, MCV was 106 fl, and white cell count was 9500/μl; he did not keep his return appointment.

Until recently, the patient had few sickle cell-related problems other than a presumed crisis (diffuse bone pains) with antibiotic-treated pneumonia at the age of 12 years. In the past 9 months, however, he required hospitalization elsewhere twice for presumed crises with pneumonia, treated with antibiotics. He was taking no medications other than daily folic acid. He denied drug or alcohol abuse and had no neurological symptoms.

Physical examination was unremarkable except for fever (102.3° F.), which remitted spontaneously later that day, and a soft, apical, holosytolic murmur. No neurological abnormalities were described but he admitted to depression. His chest x-ray showed a small effusion at the right base. Laboratory data were remarkable for the following: Hgb was 8.5 g/dl, MCV 115 fl, reticulocytes 83,200/μl (4.2%), RDW 21.9%, white cells 15,200/μl (64% neutrophils, 5% eosinophils, and 29% lymphocytes); electrophoresis showed 51.2% Hgb S and 48.8% Hgb C; lactate dehydrogenase was 432 U/l; glucose levels were normal and cultures of blood and urine were negative. Additional studies done because of his worsening macrocytosis showed a low serum cobalamin (111 pmol/l), high methylmalonic acid (1840 nmol/l) and homocysteine (125.9 umol/l), a positive antibody to intrinsic factor, and elevated serum gastrin (631 ng/l). Red blood cell folate was elevated (1432 nmol/l) and serum ferritin was 145 μg/l.

The patient's striking metabolic changes confirmed that his low cobalamin level and progressive macrocytic anemia represented clinically relevant cobalamin deficiency. His positive intrinsic factor antibody, supported by a high gastrin level, established pernicious anemia (PA; defined as cobalamin malabsorption resulting from loss of gastric intrinsic factor) as its cause. This diagnosis should be considered whenever anemia worsens, MCV rises, or neurologic or mental status changes in a patient with sickle cell disease.

The patient received 100-μg cyanocobalamin injections on two consecutive days and was discharged without folic acid. He was lost to follow-up thereafter, but contact reestablished in 2008 elicited the following information. He had continued his monthly cobalamin injections and restarted folic acid after leaving the hospital. Despite hospitalization elsewhere for one more crisis in 2003 and again in 2004, he regained weight (weight loss is not uncommon in PA), was no longer depressed, and soon recovered his premorbid crisis-free existence. On his doctor's advice in 2007, he changed from injections to weekly oral doses of 500 μg cobalamin (the popularity of oral cobalamin treatment has come with reduced precision about doses, schedules, and durations appropriate to the cause of deficiency). His mother was told she too was cobalamin-deficient and received monthly cobalamin injections. In 2008, his hemoglobin level was 12.3 g/dl and MCV was 79 fl (iron status was normal), which are typical for uncomplicated SC hemoglobinopathy. Neurological examination was normal; he was bright, cheerful, and alert. He was given a cobalamin injection and advised to increase his oral cobalamin doses to 1000 μg daily or restart monthly injections (the popularity of oral cobalamin treatment has increased imprecision among physicians and patients about appropriate doses, schedules, and durations). He was also advised to obtain referral from his physician for endoscopy, because of the risk of gastric cancer and carcinoid tumors associated with PA, and for annual thyroid function testing because thyroid antibody was detected (thyroid function was normal) and the risk of thyroid dysfunction is increased in PA.

### Patient 2

A 32 year-old black man had recurrent hospitalizations over more than a decade for crises, transfusions, and other complications of his hemoglobin SS disease. His past history included frequent otitis media when younger, depression, cholecystectomy, aplastic crisis, and acute chest syndrome that required exchange transfusion. There was no known family history of cobalamin problems. He took folic acid daily and required frequent pain medications. In 1998, hydroxyurea was started but he frequently resisted medical testing and advice.

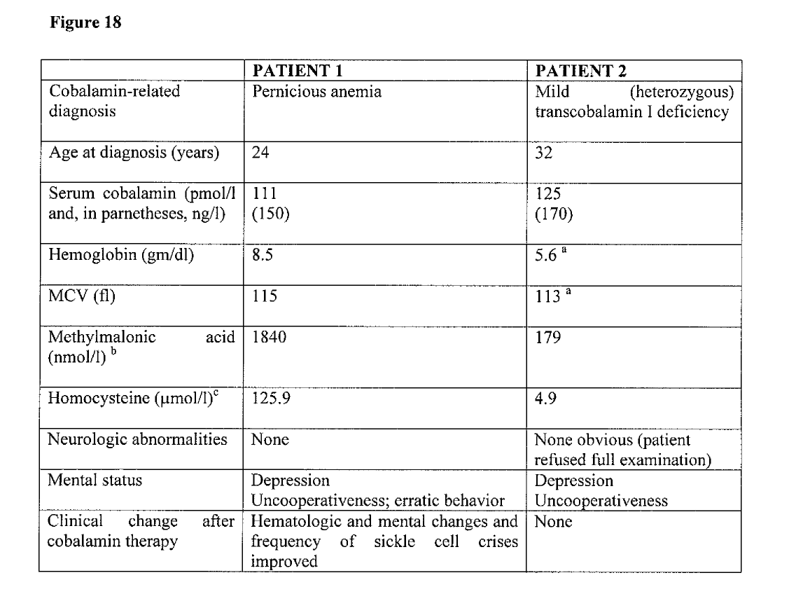

During an admission for pain crisis in 1999, brief work up for macrocytosis (hemoglobin 5.6 g/dl, MCV 113 fl) revealed serum cobalamin to be low (125 pmol/l) while serum and red cell folate levels were elevated. Physical examination was consistent with pain crisis, and he refused more than cursory neurological examination. Normal methylmalonic acid (179 nmol/l) and homocysteine levels (4.9-5.5 pmol/l) ruled out cobalamin deficiency (FIG. 18). Serum ferritin was elevated (2922 μg/l), probably reflecting his history of many blood transfusions. Antibody to intrinsic factor was absent and serum gastrin was normal, which made PA unlikely. Radioimmunoassay 15-17 showed his plasma total transcobalamin I (TC I; also called haptocorrin) concentration to be 68 pmol/l (normal=165-454 pmol/l); salivary TC I content was normal. These findings are typical for mild TC I deficiency.17 Two injections of 1000 [symbol] g of cyanocobalamin were given before TC I assays were completed and he was discharged, taking folic acid, hydroxyurea, and pain medications. The patient refused further cobalamin injections and added oral multivitamins containing cobalamin to his folic acid regimen.

Monitoring later that year showed occasional improved cobalamin levels (297-370 pmol/i) as a result of the cobalamin injections but no signs of clinical, psychological, or laboratory improvement. His hydroxyurea-induced macrocytosis and hypersegmented neutrophils persisted despite cobalamin injections. Plasma TC I remained low (59-131 pmol/l). The genetic nature of his TC I deficiency was later confirmed by DNA sequencing, which uncovered heterozygosity for a nonsense mutation (315C>T) of the TCN1 gene that is associated with TC I deficiency (he was included in that genetic study of 3 families).24

Hydroxyurea was discontinued because of apparent ineffectiveness in 2001; transfusion use, which had diminished somewhat after 2000, returned in 2004-2005 to its 1997-1999 levels. By 2006, he had chronic hepatic dysfunction, probably primarily from iron overload caused by his heavy transfusion history (serum ferritin>8000 μg/l). Pulmonary hypertension was noted in 2008. He takes folic acid and multivitamins and has had no cobalamin injections since 1999 but continues resisting most medical advice. In 2009, off hydroxyurea since 2001, his hemoglobin level was 5.2 g/dl and MCV was 93 fl.

### Discussion

Both patients had low serum cobalamin levels, but the low levels had distinctively different origins, clinical impacts, and prognostic implications. To present the diverse lessons they provide, the discussion will focus first on the association between sickle cell disease and PA and then on the association between sickle cell disease and TC I deficiency. Finally, broader diagnostic lessons will be suggested on distinguishing the low cobalamin levels of PA from those of TC I deficiency, which appears to have increasingly important clinical relevance.

### Sickle Cell Disease and PA (Patient 1)

The patient's clinical (megaloblastic anemia) and metabolic evidence of cobalamin deficiency combined with a positive intrinsic factor antibody established the diagnosis of PA. His is the fourth published case of PA occurring with sickle cell disease.^{D-F} This suggests that sickle cell disease and PA coexist too often to be coincidental. The explanation for the association is unknown but greater awareness may uncover more cases of PA among patients with sickle cell disorders in the future because PA affects young adults proportionately more frequently among blacks than whites (19 of 100 black patients with PA were <40 years old vs only 5 of 115 white patients; p<0.01).^{I} The frequency of PA in blacks, especially young women, approaches even that in elderly whites,^{I,C} and there is no reason to think blacks with sickle cell disease are exempt. However, frequencies of PA in blacks with and without sickle cell disease have not been compared prospectively, a task made more difficult nowadays because the Schilling test is no longer available.^{AI}. The PA seen in black women occurs disproportionately often at a young age,^{B,I,C} but this report indicates the finding is not limited to women.

The diagnosis of PA has typically been delayed in patients with sickle cell disease, whose chronic anemia is itself often macrocytic. The delay is a matter of concern because it carries the increased risk of neurological irreversibility,^{A} which might be further exacerbated by the nearly universal use of folic acid supplements in sickle cell disease. Two earlier patients with sickle cell disease and PA had neurological symptoms,^{E,F} which were dramatic in one case but reversed after cobalamin therapy.^{F} Fortunately, patient 1 showed no obvious neurological symptoms, reflecting the likelihood that individual predispositions to neurological defects differ. Neurological expression seems to vary inversely with hematological severity,^{K,L} and the two predispositions are accompanied by metabolic differences.^{M} Nevertheless, routine folic acid supplementation in sickle cell disease should be reevaluated. The marked improvement in folate status since the American diet became folic acid-fortified^{N} may have made routine supplementation superfluous. At the very least, the possibly increased coexistence of PA suggests the need for annual screening of cobalamin levels in sickle cell patients taking folic acid.

The duration of delayed diagnosis of cobalamin deficiency in patient 1 is unknown, but it may have been a year or longer, as suggested by his 30-lb weight loss, a well known, unexplained manifestation of untreated PA.^{A} H is several hospitalizations with acute chest syndrome and crises in the 9 months leading up to his hospitalization contrast with his nearly crisis-free existence both before the onset of PA and after institution of cobalamin treatment.

### Sickle Cell Disease and TC I Deficiency (Patient 2)

TC I (also called haptocorrin, R binder, and cobalophilin, although the gene is called TCN1) is a cobalamin-binding glycoprotein found in plasma and other fluids, such as saliva and breast milk, and elaborated by myeloid cells and glandular epithelial cells.^{T} Its function is unknown. Because it does not undergo specific uptake by cells, plasma TC I has a very slow turnover and, thus, typically carries >80% of circulating cobalamin, including all nonfunctional cobalamin analogs.^{AE} The metabolically important fraction of cobalamin carried by TC II, which is rapidly taken up by cells, constitutes <20% of circulating cobalamin.

These facts explain why the absence of TC I-bound cobalamin (holo-TC I) causes a low serum cobalamin level without impairing cellular cobalamin status, which depends on TC II-cobalamin (holo-TC II).^{T} The asymptomatic nature of TC I deficiency explains why the disorder is typically identified accidentally by its low serum cobalamin level, often in old age, and why it is commonly mistaken for cobalamin deficiency. Many patients are treated unnecessarily with cobalamin for years. The serum cobalamin levels rise slightly and transiently after cobalamin injection but sometimes approach normal if circulating holo-TC II rises sufficiently, as seen in patient 2. Clinical or metabolic benefit from cobalamin therapy is not seen,^{P,J,O,W} except in rare cases whose TC I deficiency coexists with a disorder that causes cobalamin deficiency.^{S}

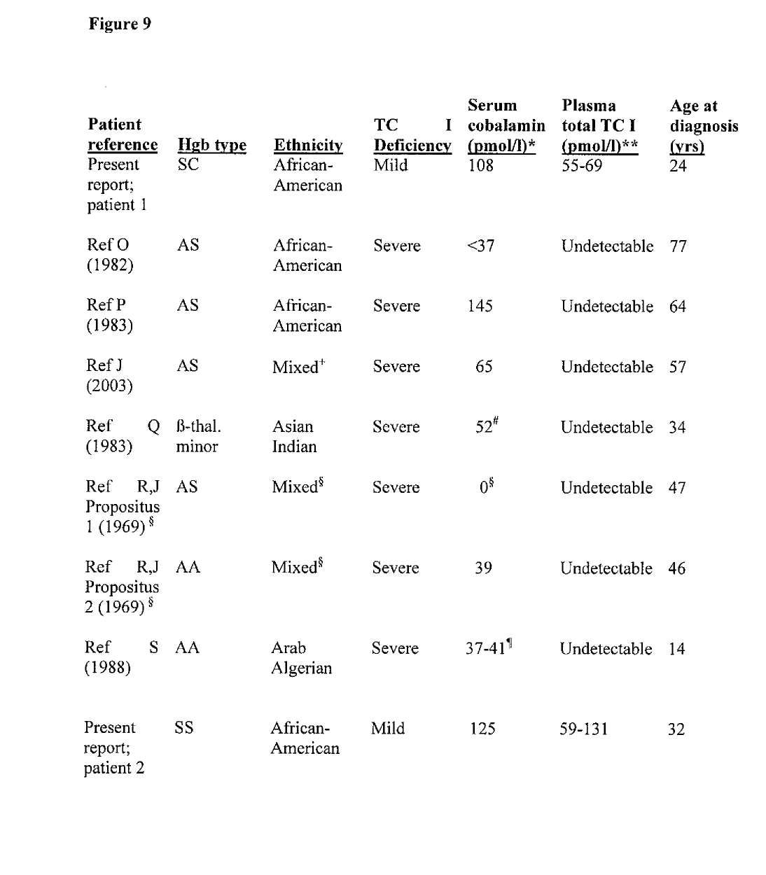

TC I deficiency has been thought rare, an underestimate explained by its asymptomatic nature, its usually indirect detection via a low serum cobalamin, and the general unavailability of reliable tests to diagnose it. Clinical laboratories measure unsaturated (i.e., apo form) cobalamin-binding capacity, rather than total transcobalamins. Such tests do not distinguish between TC I and TC II and reflect TC I status poorly because most plasma TC I is holo-TC I; the assays also produce falsely low results in blood sampled after cobalamin therapy, which saturates apo-TC I and apo-TC II. Immunoassay of total (i.e., apo and holo) TC I is available only in research laboratories. Patient 2, like other reported cases (FIG. 9), was eventually diagnosed after the initial misinterpretation of his low cobalamin level was reevaluated because symptoms, signs, and metabolic indicators of cobalamin deficiency were lacking. His low TC I level suggested the diagnosis of mild TC I deficiency) rather than severe TC I deficiency, in which plasma TC I is undetectable (FIG. 9). PCR analysis of his TCN1 gene provided definitive proof of a genetic explanation by demonstrating heterozygosity for a nonsense mutation, as discovered in TC I-deficient members of other families (see, e.g., Example 1).

It is interesting that all cases of severe TC I deficiency, in which TC I is absent, have been identified in non-Caucasians to date (FIG. 9). All but two^{Q,S} have been African-American or had partially African origin, and one of the two exceptions was a northern African Arab.^{S} This ethnic preponderance is somewhat paradoxical, given that blacks have significantly higher plasma TC I and cobalamin levels than do whites or Asians.^{H,U,V,AF} Equally striking, and extended by the findings in patient 2, is the frequent association of TC I deficiency with sickle cell and other mutations of the β-globin gene (FIG. 9). Of 7 published cases of severe TC I deficiency, 4 patients unrelated to each other had sickle cell trait and a fifth had β-thalassemia minor, whereas the prevalence of sickle cell trait is approximately 8% among American blacks and that of sickle cell anemia is <0.2%.^{AJ} Patient 2 extends the association, although his TC I deficiency is mild and he has sickle cell disease, not the trait.

With the case reported here, 6 of 8 TC I-deficient patients have had mutations of the β-globin gene. Although both the TC I and β-globin genes are on chromosome 11, their locations are far apart (11q12-13 and 11p15, respectively) and the allele dosages appear not to match in most patients; this makes linkage dysequilibrium an unlikely explanation. The coexistence could be a statistical quirk but study selection bias is unlikely because virtually all black subjects with TC I deficiency have undergone hemoglobin electrophoretic testing. The association may reflect a selection pressure, for example, favoring TC I deficiency in the subpopulation with a heavy load of sickle cell hemoglobin mutation.

### Distinguishing Between TC I Deficiency and Cobalamin Deficiency (FIG. 10)

As long as TC I deficiency was considered a rare curiosity, diagnosing it was a low medical priority. However, its mimicking of cobalamin deficiency now appears to have substantial medical relevance for two reasons. One reason is that prospective and retrospective studies found mild TC I deficiency to be associated with 15% of all unexplained low cobalamin levels and severe TC I deficiency with 0.6% of low cobalamin levels.^{J} (Although a later report attributed low TC I levels to cobalamin deficiency,^{AG} the study described very few subnormal total TC I levels and had substantial shortcomings.^{AH}) The other reason for medical relevance is the high frequency of unexplained low cobalamin levels in the general population.^{X-Z} Many persons have asymptomatic, subclinical, and usually nonmalabsorptive cobalamin deficiency,^{Z,AD} which is difficult to differentiate clinically from TC I deficiency. Falsely low cobalamin levels (i.e., lacking all metabolic evidence of cobalamin deficiency) are common and many of these may reflect TC I deficiency.^{J}

The misdiagnosis of TC I deficiency as cobalamin deficiency and unnecessary cobalamin therapy, as occurred in patient 2 and most published cases of TC I deficiency,^{O,P,Q,R,S} and the clinical unavailability of reliable tests for TC I deficiency were discussed in the previous section. Less frequent but more serious in their consequences are misdiagnoses of cobalamin deficiency as TC I deficiency. An example is a case report^{AA} of “TC I deficiency” diagnosed by poor methods, which prompted discontinuation of cobalamin therapy despite the patient's mild macrocytosis and paresthesias that improved after cobalamin therapy. TC I deficiency requires no cobalamin therapy, but cobalamin deficiency, especially when arising from PA and other malabsorptive causes of clinical cobalamin deficiency, requires urgent cobalamin treatment.^{AB}

TC I deficiency should be considered in all patients with low cobalamin levels who lack unequivocal clinical or metabolic signs of cobalamin deficiency. Proper diagnosis of mild TC I deficiency also requires rapid separation and processing of plasma; serum, which is subject to artifactual release of TC I from granulocytes in vitro,^{AC} must not be used. However, because TC I levels may be borderline in some patients with proven mild TC I deficiency,^{J} genomic DNA was examined in patient 2, who had mild TC I deficiency, and in families with proven severe or mild TC I deficiency. These studies confirmed TCN1 mutations that are characteristic of the respective TC I deficiencies (see Example 1). Genetic testing may be a reliable tool to identify TC I deficiency, in order to determine who may benefit from cobalamin therapy and who will not, and to prevent unnecessary or incorrect therapy. It will also be applicable in population surveys and studies of associations with other conditions.

Despite similarly low cobalamin levels (FIG. 18), the cobalamin-related conditions in the two cases have distinctively different origins, clinical impacts, prognoses, and management strategies. The clinical relevance of distinguishing the low cobalamin levels of PA from those of TC I deficiency extends beyond sickle cell disease but evidence of a special resonance for sickle cell disease is accumulating. Patient 1 is the fourth reported case of PA complicating sickle cell disease, suggesting the combination is not rare. Recognition of superimposed PA and other irreversible causes of cobalamin malabsorption has special urgency in the face of the presently routine folate supplementation in sickle cell disease in the era of folic acid fortification.

Cobalamin levels are also often low for reasons other than PA, however, and many may not require cobalamin treatment. The task of distinguishing them can be difficult, but it must be pursued in order to avoid unnecessary cobalamin treatment in many cases while assuring that the few who need cobalamin urgently receive proper treatment. TC I deficiency, which might be disproportionately associated with sickle cell trait and disease (FIG. 9), is one such cause of a low cobalamin level mimicking that of PA but not requiring cobalamin treatment.

### REFERENCES FOR EXAMPLE 2

A. Chanarin I. The Megaloblastic Anaemias. 2^{nd} ed. Blackwell Scientific Publ, Oxford. 1979.
B. Carmel R. Prevalence of undiagnosed pernicious anemia in the elderly. Arch Intern Med 1996; 1097-1100.
C. Carmel R, Johnson C S. Racial patterns in pernicious anemia: early age of onset and increased frequency of intrinsic factor antibody in black women. N Engl J Med 1978; 298:647-50.
D. Chen M-C, Koshy M, Kennedy J. Pancytopenia caused by unsuspected pernicious anemia complicating sickle cell-β thalassemia. South Med J 1992; 85:215-216.
E. Sinow R M, Johnson C S, Karnaze D S, Siegel M E, Carmel R. Unsuspected pernicious anemia in a patient with sickle cell disease receiving routine folate supplementation. Arch Intern Med 1987; 147:1828-9.
F. Dhar M, Bellevue R, Carmel R. Pernicious anemia with neuropsychiatric dysfunction in a patient with sickle cell anemia treated with folate supplementation. N Engl J Med 2003; 348:2204-7.
G. Lin J C, Borregaard N, Liebman H A, Carmel R. Deficiency of the specific granule proteins, R binder/transcobalamin I and lactoferrin, in plasma and saliva: a new disorder. Am J Med Genet. 2001; 100:145-51.
H. Carmel R, Brar S, Frouhar Z. Plasma total transcobalamin I: ethnic/racial patterns and comparison with lactoferrin. Am J Clin Pathol 2001; 116:576-80.
J. Carmel R, Johnson C S, Weiner J M. Pernicious anemia in Latin Americans is not a disease of the elderly. Arch Intern Med 1987; 147:1995-6.
J. Carmel R. Mild transcobalamin I (haptocorrin) deficiency and low serum cobalamin concentrations. Clin Chem 2003; 49:1367-74.
K. Magnus E M. Cobalamin and unsaturated transcobalamin values in pernicious anaemia: relation to treatment. Scand J Haematol 1986; 36:457-65.
L. Savage D, Lindenbaum J. Relapses after interruption of cyanocobalamin therapy in patients with pernicious anemia. Am J Med 1983; 74:765-72.
M. Carmel R, Melnyk S, James S J. Cobalamin deficiency with and without neurologic abnormalities: differences in homocysteine and methionine metabolism. Blood 2003; 101:3302-8.
N. Joelson D W, Fiebig E W, Wu A H B. Diminished need for folate measurements among indigent populations in the post folic acid supplementation era. Arch Pathol Lab Med 2007; 131:477-80.
O. Carmel R. A new case of deficiency of the R binder for cobalamin, with observations on minor cobalamin-binding proteins in serum and saliva. Blood 1982; 59:152-6.
   P. Carmel R. R binder deficiency: a clinically benign cause of cobalamin pseudo-deficiency. JAMA 1983; 250:1886-90.
Q. Jenks J, Begley J, Howard L. Cobalamin R binder deficiency in a woman with thalassemia. Nutr Rev 1983; 41:277-80.
R. Carmel R, Herbert V. Deficiency of vitamin B₁₂-binding alpha globulin in two brothers. Blood 1969; 33:1-12.
S. Zittoun J, Léger J, Marquet J, Carmel R. Combined congenital deficiencies of intrinsic factor and R binder. Blood 1998; 72:940-3.
T. Carmel R. Cobalamin-binding proteins in man. In: Contemporary Hematology-Oncology. Silber R, Gordon A S, LoBue J, Muggia F M (eds.), Vol. 2, Plenum, New York, 1981, p. 79-129
R. U. Fleming A F, Ogunfunmilade Y A, Carmel R. Serum vitamin B₁₂, unsaturated vitamin B₁₂-binding capacity and transcobalamins in Nigerians and Europeans. Am J Clin Nutr 1978; 31:1732-8.
V. Carmel R. Ethnic and racial factors in cobalamin metabolism and its disorders. Semin Hematol 1999; 36:88-100.
W. Hall C A, Begley J A. Congenital deficiency of human R-type binding proteins of cobalamin. Am J Hum Genet. 1977; 29:619-26.
X. Lindenbaum J, Rosenberg I H, Wilson P W F, Stabler S P, Allen R H. Prevalence of cobalamin deficiency in the Framingham elderly population. Am J Clin Nutr 1994; 60:2-11.
Y. Carmel R, Green R, Jacobsen D W, Rasmussen K, Florea M, Azen C. Serum cobalamin, homocysteine and methylmalonic acid concentrations in a multiethnic elderly population: ethnic and sex differences in cobalamin and metabolite abnormalities. Am J Clin Nutr 1999; 70:904-10.
Z. Carmel R. Current concepts in cobalamin deficiency. Annu Rev Med 2000; 51:357-75.
AA. Adcock B B, McKnight J T. Cobalamin pseudodeficiency due to transcobalamin I deficiency. South Med J 2002; 95:1060-2.
AB. Carmel R. How I diagnose and treat cobalamin (vitamin B₁₂) deficiency. Blood 2008; 112:2214-21.
AC. Carmel R. Vitamin B₁₂-binding proteins in serum and plasma in various disorders. Effect of anticoagulants. Am J Clin Pathol 1978; 69:319-25.
AD. Carmel R, Sinow R M, Karnaze D S. Atypical cobalamin deficiency: subtle biochemical evidence of deficiency is commonly demonstrable in patients without megaloblastic anemia and is often associated with protein-bound cobalamin malabsorption. J Lab Clin Med 1987; 109:454-63.
AE. Carmel R. The distribution of endogenous cobalamin among cobalamin-binding proteins in the blood in normal and abnormal states. Am J Clin Nutr 1985; 41:713-9.
AF. Saxena S, Carmel R. Racial differences in vitamin B₁₂ levels in the United States. Am J Clin Pathol 1987; 88:95-7.
AG. Morkbak A L, Hvas A M, Lloyd-Wright Z L, Sanders T A B, Blele O, Refsum H, et al. Effect of vitamin B12 treatment on haptocorrin. Clin Chem 2006; 52:1104-1111.
AH. Carmel R. Haptocorrin (transcobalamin I) and cobalamin deficiencies. Clin Chem 2007; 53:367-368.
AI. Carmel R. The disappearance of cobalamin absorption testing: a critical diagnostic loss. J Nutr 2007; 137:2481-2484.
AJ. Wang W C. Sickle cell anemia and other sickling syndromes. In: Wintrobe's Clinical Hematology. 12th ed. Greer J P, Foerster J, Rodgers G M, Paraskevas F, Glader B, Arber D A, Means R T Jr, editors. Lippincott Williams & Wilkins: Philadelphia. 2009; 1038-1082.

### Example 3

### A TCN1 Mutation in Exon 6 is Associated with Transcobalamin I Deficiency and Low Cobalamin Levels and May be the Most Common Hereditary TC I Deficiency, Especially in Patients of European Origin

Deficiency of transcobalamin (TC) I, a protein that carries most of the cobalamin in the blood but whose function is unclear, causes low serum cobalamin levels that are indistinguishable from those of cobalamin deficiency. The diagnosis can be difficult to establish and is typically missed, leading to unnecessary long-term treatment as cobalamin deficiency. Having found two new mutations in exon 2 of the TCN1 gene that were responsible for both severe and mild TC I deficiency in 3 unrelated families of African, Hispanic, and Mediterranean origin (see Example 1)^{J}, genotyping was extended to new TCN1 mutations in other patients with mild TC I deficiency and mildly low cobalamin levels. Four unrelated propositi of European ancestry were identified whose TC I deficiency was associated with a 999G>T mutation in exon 6, causing the substitution of tyrosine for aspartic acid in the highly conserved amino acid 301 position. All 4 TC I-deficient relatives of the propositi also had the mutation, whereas all 4 non-deficient relatives had no mutation. Heterozygosity for the 999G>T mutation has been posted on the NCBI website as occurring in 11.6±0.2% of the general population, especially in whites (21.7-29% frequency of heterozygosity). However, its association with TC I deficiency was not recognized. The findings described herein, combined with the allele frequencies suggest that mild hereditary TC I deficiency may be common, especially in whites.

### Introduction

Transcobalamin I (TC I; also called haptocorrin) originates in the specific granules of myeloid precursors and in exocrine gland epithelial cells. Its function, unlike that of the two other cobalamin-binding proteins, TC II and intrinsic factor, is still uncertain. Deficiency of TC I has been thought rare because case reports have been few and largely limited to severe deficiency,^{A-E} because TC I assay is rarely performed, and most especially because TC I deficiency produces no definitive sequelae other than a low serum cobalamin level. The low serum cobalamin level occurs because >70% of cobalamin normally circulates attached to TC I in the bloodstream,^{F-H} but cellular cobalamin content, metabolically defined cobalamin status, and clinical cobalamin status have been normal in TC I-deficient subjects.^{A-D,F,I}

The low serum cobalamin in TC I deficiency is typically uncovered accidentally and often mislabeled and treated as cobalamin deficiency. This is abetted by the general unavailability of reliable diagnostic tools and the self-fulfilling assumption that TC I deficiency is rare, but also by the fact that cobalamin assay is often requested in cases with coincidental manifestations that vaguely resemble those of cobalamin deficiency.

As described in Example 1, two mutations in exon 2 of the TCN1 gene that lead to a premature stop codon have been identified.^{J} Severe TC I deficiency, with undetectable TC I in both plasma and secretions, such as saliva, and serum cobalamin levels <100 pmol/l, occurred when both alleles were affected by either of the exon 2 mutations. Mild TC I deficiency, with mildly low plasma TC I and cobalamin levels and normal saliva TC I, occurred when only one allele was affected, which is predictably more common than the homozygous state required for severe TC I deficiency. These genetic findings were extended to subjects in whom phenotypically mild TC I deficiency was confirmed and in whom cobalamin deficiency was excluded by metabolic tests such as methylmalonic acid and homocysteine, but who did not have the exon 2 mutations. Full genotyping of their TCN1 gene uncovered a new genetic association in four families who, unlike the previous three families in Example 1 with the exon 2 mutations, are whites of European origin.

### Patient Characterization

### Family 1

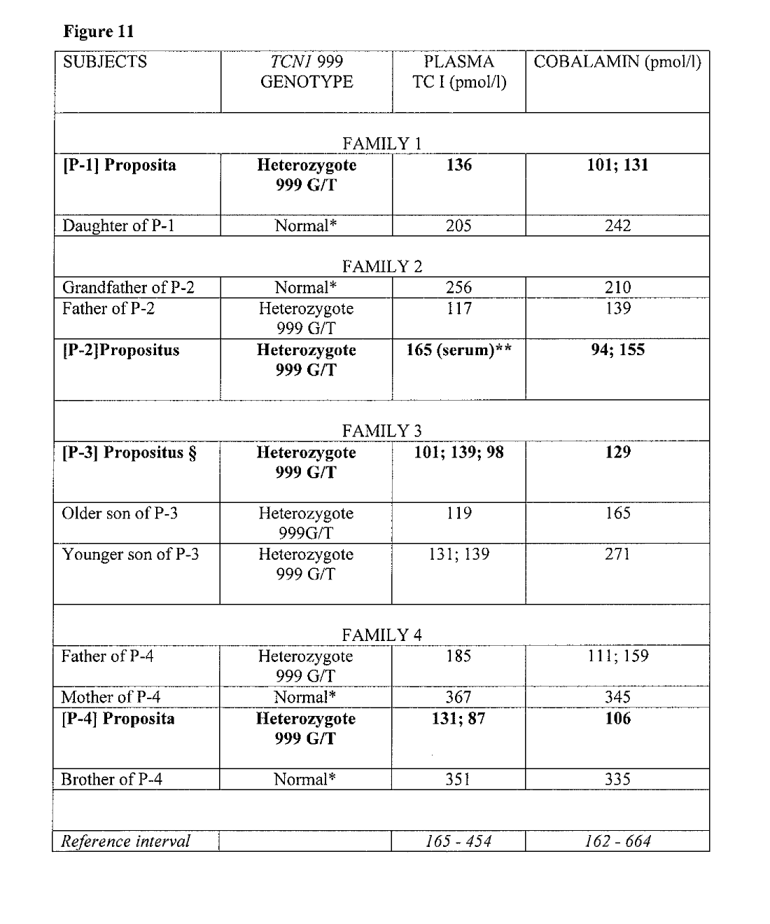

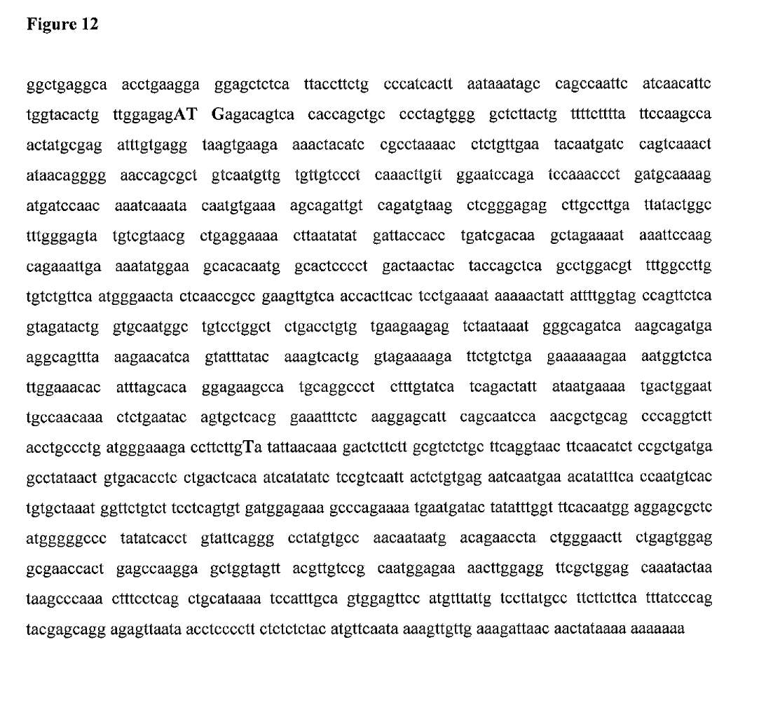

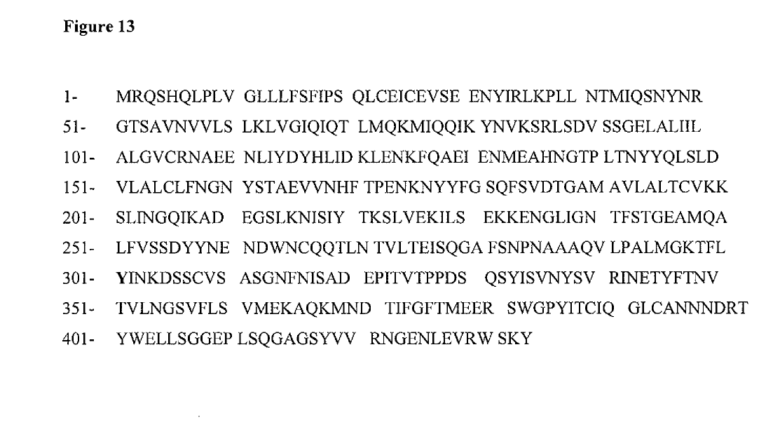

P-1, the 50 year old asymptomatic and hematologically and neurologically normal proposita, was found to have a low serum cobalamin 5 years earlier when her blood was ‘screened’ in the clinical laboratory where she worked; the most recent value was 101 pmol/l. Methylmalonic acid (MMA) and homocysteine levels and the Schilling test result were normal. Her medical history was unremarkable except for treated hypothyroidism and iron deficiency because of frequent blood donation. At least one relative was said to have “pernicious anemia,” but is deceased so it is not known if TC I deficiency, rather than pernicious anemia, caused his low cobalamin level. The proposita had undergone oral cobalamin therapy followed by monthly injections, which raised serum cobalamin but produced no metabolic or clinical changes. Testing revealed mild TC I deficiency (FIG. 11). Saliva TC I was normal.

Her 23-year old daughter was found not to have TC I deficiency. The family origins are western European.

### Family 2

P-2, the propositus, was found to have borderline MMA elevation (390 nmol/l; normal <379) at the age of 6 months during evaluation of mild acidosis and transient elevation of hepatic transaminases attributed to insecticide exposure. Serum cobalamin was then found to be 94 pmol/l (normal >250 pmol). He was not treated with cobalamin because his blood count, plasma homocysteine (6 μmol/l), and Schilling test results were normal and he was asymptomatic and had normal neurologic status and development. Eight months later, he was brought for evaluation of the low cobalamin level, which was still low (155 pmol/l in our laboratory). TC I was 165 pmol/l in serum (normal, 234-557 for serum; the patient's blood sampling was limited to 1 ml serum, which provides spuriously higher levels than plasma)^{I} and saliva TC I was low-normal and tear TC I was detectable but lower than in a single control subject; these findings indicated mild TC I deficiency.^{I,J} The child's subsequent development was normal, without cobalamin therapy, and 15 years later he is a good student and athlete. It is now also known that mild MMA elevation, such as he exhibited at 6 months, is common in children during the first year of life and reverses spontaneously;^{M} cobalamin levels as low as his, however, are uncommon at that age.

The child's father, a military pilot in good health and with normal hematologic and metabolic findings, was found to have mild TC I deficiency and mildly low serum cobalamin level (FIG. 11). His metabolic and absorption testing, done in lieu of testing his infant son, were completely normal, including deoxyuridine suppression, Schilling test, and food-bound cobalamin absorption testing. The family origins were primarily western European, with some Slavic, American Indian, and Jewish ancestry.

### Family 3

P-3, the propositus, was found to have a low serum cobalamin level (129 pmol/l) at the age of 44 years during evaluation of his chronic dysphonia (central bowing of the vocal cords was the only finding). He also described “heaviness” of arms and legs and numbness and tingling, although neurologic testing was negative, and noted slight improvement with cobalamin therapy. Despite a completely normal blood count, including MCV, and Schilling test, pernicious anemia was suspected when intrinsic factor antibody was reported in his blood (proven to be a cobalamin-induced artifact upon retesting with a new blood sample after cobalamin injections were discontinued). Reevaluation after discontinuing cobalamin therapy also showed normal neutrophil segmentation, MMA level, holo-transcobalamin level, and food-bound cobalamin absorption, and confirmed the normal Schilling test results. Plasma TC I was mildly decreased (101 pmol/l) and saliva TC I was normal, consistent with mild TC I deficiency. Laryngoplasty corrected his dysphonia.

The family was of Ashkenazi Jewish ancestry. Testing revealed mild TC I deficiency in one son (FIG. 11); his serum MMA level was normal, indicating normal cobalamin metabolism, and his medical history was positive only for alcohol abuse. An older son had low plasma TC I (119 pmol/l) and cobalamin (165 pmol/l). He died of drug overdose but DNA was extracted from an archived serum specimen (FIG. 11).

### Family 4

P-4, a 25-year old woman, was found to have a low cobalamin level (106 pmol/l) during evaluation for a 2-year history of cognitive and memory decline that had led to the loss of her job. The onset corresponded temporally with a cervical spine fusion during which nitrous oxide may have been used, several months after a car accident. She also had presumptive infectious mononucleosis (Epstein-Barr positive) not long after her symptoms began, and she underwent a 6-month, 5-drug course of eradication of Helicobacter pylori which was associated with recurrent ulcers since high school. Extensive neurological work-up, including MRI, cerebral positron-emission tomography, electroencephalography, and lumbar puncture, was unrevealing. A Schilling test result was normal. Cobalamin injections coincided with subjective improvement that proved transient. A neurological examination showed only equivocal vibratory diminution and Romberg sign. Testing revealed mild TC I deficiency (plasma TC 1131 pmol/l; normal saliva TC I). Normal results included MMA, homocysteine, blood count including MCV, and lactoferrin. A several month course of semi-weekly hydroxocobalamin injections did not produce notable improvement. She subsequently underwent intensive neuropsychiatric evaluation and treatment, receiving diagnoses at different times of hippocampal seizures and congenital adrenal hyperplasia for which hormonal therapy was given. After 10 years, she remains cognitively limited but has been helped somewhat by daily doses of dextroamphetamine and amphetamine salts and continues taking daily 1000 μg cobalamin orally although a diagnosis and objective evidence of benefit is lacking. She completed professional retraining, works, is married, and has 2 children.

Her father, who has diabetes mellitus, was found to have mild TC I deficiency too (FIG. 11) and has no metabolic evidence of cobalamin deficiency. Her mother, whose hypothyroidism is under medical control, and a brother have normal plasma TC I and cobalamin levels. The family is of western European origin.

### Methods

The genetic studies were approved by the Institutional Review Board and subjects who donated leukocytes gave written consent for DNA testing. The search of the TCN1 gene^{K,L} for mutations in the coding region used the previously described set of 9 primer pairs covering exons 1-9, which were designed from intronic sequences encompassing each exon (see FIG. 2)^{J} PCR reactions were performed with either Go-Taq (Promega, Madison, Wis.) or Pfu polymerase (Stratagene, La Jolla, Calif.). Following amplification, the products were cloned into either pCR2.1-Topo or pCR4 (Invitrogen, Carlsbad, Calif.). Sequencing was done at the Bioresource Center DNA Sequencing Facility, Cornell University (Ithaca, N.Y.). The sequences were compared to the known TCN1 sequence (NM_{—}001062).

### Results

The mildly TC I-deficient propositi in all four families were heterozygous for a G>T substitution at nucleotide 999 (rs34324219) in the TCN1 gene (FIG. 11). Patients P-1 and P-2 are typical cases of mild TC I deficiency, whose cobalamin levels were tested accidentally; the testing in P-2 were done because MMA elevation was not known at the time to be common but benign and transient in the first year of life^{M}. The dysphonia that prompted cobalamin testing in patient P-3 is not a known manifestation of cobalamin deficiency and turned out to have unrelated causes; nevertheless, he was treated with cobalamin for several years because of the low serum cobalamin level. Only in patient P-4 was cobalamin deficiency a viable possibility, although most of the evidence does not support the connection. Tellingly, in all 4 families, including that of patient P-4, TC I-deficient relatives of the propositi did not have clinical pictures similar to the propositi and had no evidence of cobalamin deficiency despite having similar TC I and cobalmin levels, which suggests that the clinical issues in the propositi were not intrinsic to TC I deficiency.

The 999G>T mutation replaces the highly conserved aspartic acid by tyrosine as amino acid 301 in the 433-amino acid sequence, a substitution with potentially substantial impact on the TC I protein. None of the patients displayed mutations in other exons, including the exon 2 mutations described previously in subjects with TC I deficiency (see Example 1).^{J}

Study of the four families showed that all 4 relatives who were found to be also mildly TC I-deficient were similarly heterozygous for the 999G>T mutation, whereas all 4 relatives who were found to have normal TC I status had no mutation (FIG. 11). None of the 4 TC I-deficient relatives had ever undergone cobalamin testing previously because they had no symptoms suggestive of possible cobalamin deficiency that might prompt medical testing. None of the propositi or affected relatives had cobalamin malabsorption, anemia, metabolic changes compatible with cobalamin deficiency, or demonstrated any clinical or metabolic responses to cobalamin therapy.

The 999G>T mutation was also not found in the subjects reported previously to have TC I deficiency associated with exon 2 mutations (see Example 1).

### Discussion

The propositi and affected relatives described here met the criteria for mild TC I deficiency, which is associated with the heterozygous state as had been the case with patients who had exon 2 mutations.^{J} They had mild but persistent reduction, not absence, of plasma TC I levels with secondarily low or low-normal cobalamin levels that were shown not arise from malabsorption or dietary insufficiency, and they had normal saliva TC I. They also had neither clinical/hematologic nor metabolic stigmata of cobalamin deficiency, and cobalamin therapy made no impact. With regard to proposita P-4, although her severe neurologic deterioration was not caused by cobalamin deficiency, the question arises whether her likely exposure to nitrous oxide, which inactivates cobalamin, might have played some role. This has not been reported previously in TC I deficiency and seems unlikely, but the possibility that TC I deficiency poses a silent, latent link in cobalamin metabolism susceptible to activation under external pressure merits consideration. Alternatively, P-4 may have had an independent or perhaps indirectly linked neurological condition, much as did the second of two severely affected brothers in the original report of severe TC I deficiency, who had an unidentified neurodegenerative disorder that his equally TC I-deficient brother did not.^{A,N}

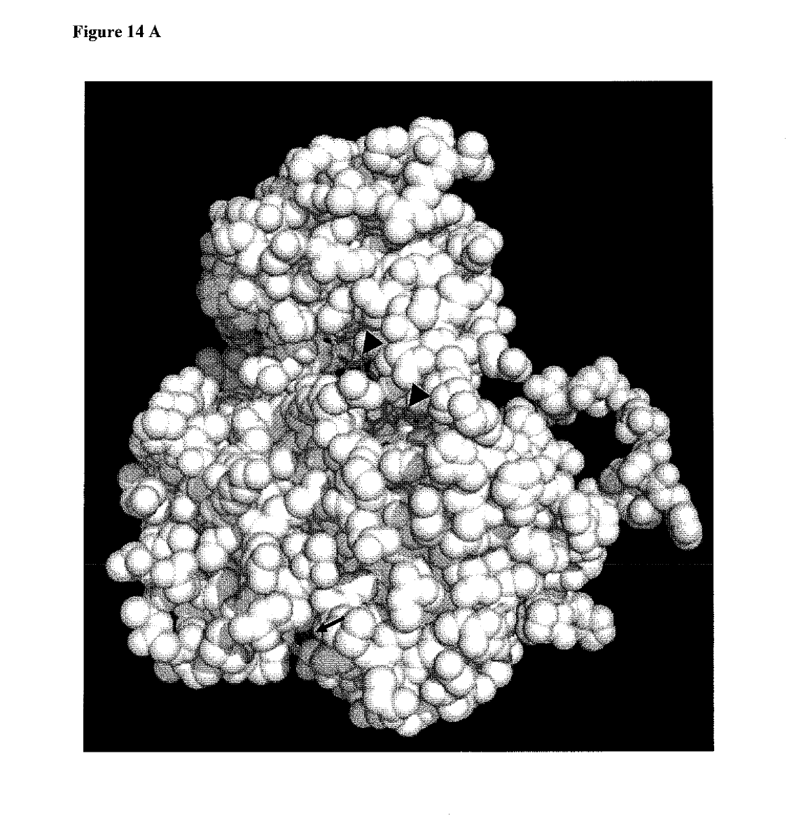

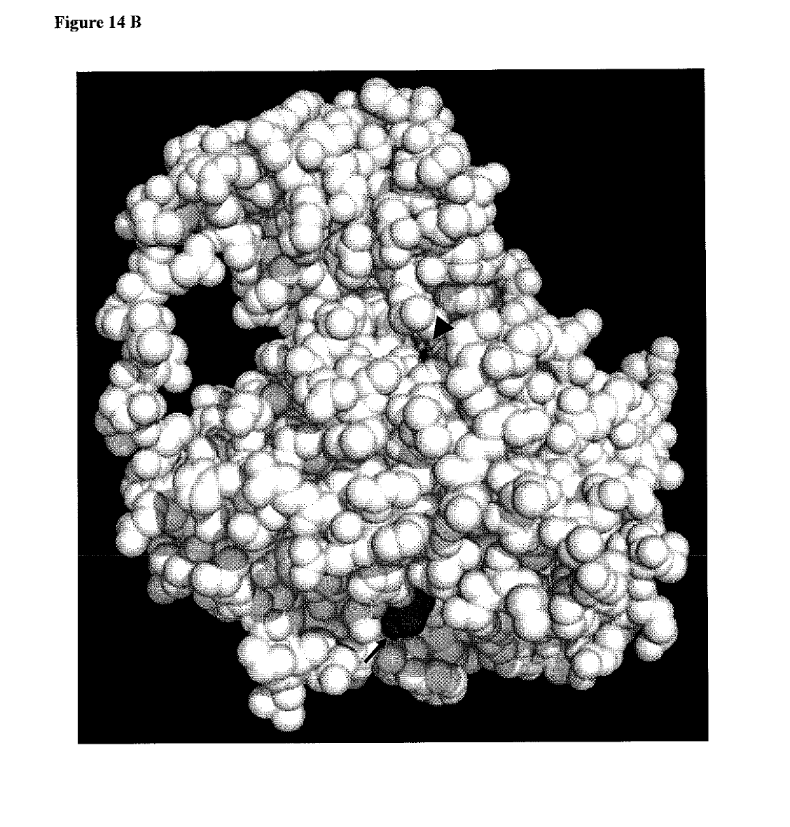

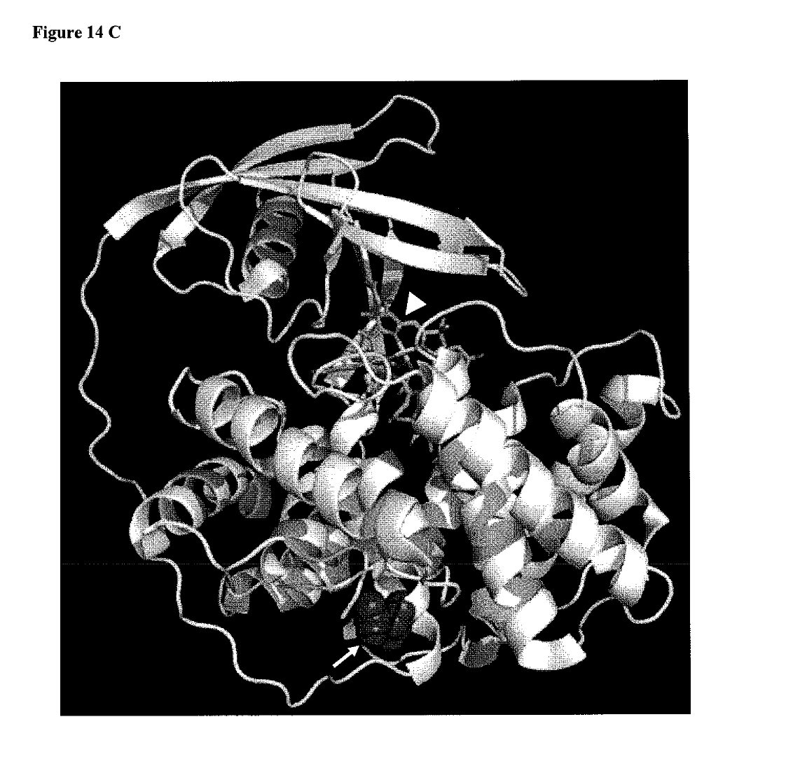

The 3-dimensional structure of the human TC I protein has not been determined, in large part because TC I is heavily glycosylated. However, the structures of human and bovine TC II proteins have been determined, and it was suggested that they can be used to predict the structure of TC I^{O,P} Using the structure of the human TC II protein (PDB ID: 2BB5) the location of the conserved Asp301 residue was mapped to the surface of the molecule, far from the putative cleft between the α and β domains to which cobalamin binds (FIG. 14). The tyrosine substitution therefore appears unlikely to directly affect cobalamin binding to TC I, although this cannot be ruled out completely. However, the substitution at this position may affect the local structure of the molecule, as some possible clashes with adjacent residues can be observed when tyrosine is present. The localization to the surface of the molecule also suggests that the substitution and subsequent structural alteration may affect the posttranslational modification (e.g., glycosylation) of TC I, perhaps thereby altering TC I hepatic or renal clearance from the circulation. Alternatively, the substitution could render the molecule unstable or affect its behavior under selected conditions.

With the present study, the TCN1 gene has now been analyzed in 11 unrelated families with TC I deficiency. Previously, two exon 2 mutations were identified, 270G deletion and 315C>T (see, Example 1), distributed among 8 affected members within 3 families.^{J} An additional severely TC I-deficient patient was also found to have the 270G deletion together with a new 475T>C mutation (see, Example 5). The present report adds 8 TC I-deficient members of 4 other families with the 999G>T mutation in exon 6. This leaves only 3 of the studied families with TC I deficiency to date in whom a TCN1 mutation has not been identified. The 4 TCN1 mutations identified may therefore account for most TC I deficiency. It also suggests ethnic distribution differences because all 999G>T mutations causing TC I deficiency occurred in patients of European origin, whereas the two exon 2 mutations appeared to be limited to patients of African, Hispanic, and Mediterranean origins (see Examples 1 and 2).

Homozygotes for the 999G>T mutation were not found in this study. Surveys posted on the NCBI website list only a 1% frequency for the homozygous mutation.^{W} Mutation of both alleles would presumably induce more severe TC I deficiency than the mild heterozygous state, but the severity is unpredictable because the 999G>T mutation produces only an amino acid substitution, unlike the nonsense mediated mRNA decay following the exon 2 mutations.

Only one 999T allele has been found among 48 alleles from 24 healthy control subjects with normal TC I (and cobalamin) levels (data not shown). This singular control subject with normal TC I status despite his 999G>T mutation was also homozygous for the rs492602 [G] and rs602662 [A] polymorphisms of FUT2, which are linked to the regulation of α,1,2-fucosyltransferase. The FUT2 mutations reduce fucosylation and homozygosity was associated with significantly increased serum cobalamin levels (mean cobalamin levels of 480-490 pmol/l versus 410 pmol/l in non-homozygotes).^{Q} Based on the known association of FUT2 with blood group substances in secretions, it was hypothesized in that report that reduced fucosylation somehow improved secretion of intrinsic factor, a gastric glycoprotein responsible for delivering cobalamin to ileal cells, reduced susceptibility to Helicobacter pylori gastritis, or had other gastrointestinal effects.^{Q,Y}

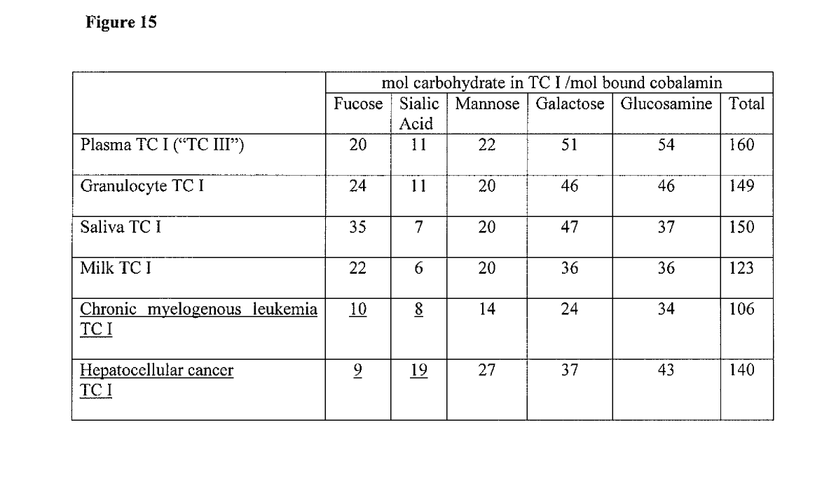

However, the change in serum cobalamin levels linked to FUT2 mutation may be directly effected by changes in fucosylation of TC I, which is a heavily glycosylated protein. Support for such a scenario lies in older studies of TC I isoforms documenting decreased fucose residues along with the more emphasized increased sialic acid residues which have been associated with slower clearance of the protein via hepatic asiologlycoprotein receptors.^{R-U} These lightly fucosylated TC I proteins (e.g., 9-10 vs. 20-24 fucose residues/mol cobalamin; see FIG. 15),^{R} which are prominent in chronic myelogenous leukemia and hepatocellular carcinoma, are cleared more slowly from the bloodstream than more fucosylated and less sialylated TC I (especially the fraction of TC I dubbed “TC III” in the past)^{R} and are associated with markedly increased (often severalfold greater) plasma TC I and cobalamin levels.^{S,T,U} The mentioned diseases with high levels of such TC I proteins with diminished fucose content are associated with markedly increased serum cobalamin levels.^{S,G} The FUT2 polymorphism may therefore induce a modest version of the same fucosylation reduction and effect on plasma clearance of TC I and on cobalamin accumulation in the general population. In the subject with the 999T allele who was not TC I-deficient (plasma TC I and cobalamin pmol/l) despite his heterozygosity for 999G>T, any mild diminution of TC I levels caused by his 999G>T mutation may have been counteracted by the effect of mildly diminished fucosylation of TC I linked to his homozygosity for the FUT2 mutation. The absence of similar homozygosity for the FUT2 mutation in nearly all the 999G>T families in this study (see later) who had low TC I levels further supports this proposal.

Data in the NCBI website^{W} suggest the relatively high frequencies of 6.9% for the 999 T allele and 11.6±0.2% for the heterozygous 999G>T state. This is the highest frequency among all TCN1 mutations in that database, save for the 944C>T polymorphism, which is a synonymous and thus functionally neutral mutation. Moreover, the 999T allele frequency was highest in Caucasians and absent or low in blacks.^{W} This ethnic pattern is consonant with the European origins of all four families presented here. It is also consonant with the absence of the 999G>T mutation and the non-Caucasian origins of all three families with exon 2 mutations.^{J} The effect of heterozygosity for the 999G>T mutation on TC I levels in our study, together with the known high frequency of heterozygosity in the general population, and even higher in whites,^{W} may help explain many low cobalamin levels, which are especially frequent in whites.^{V} It has been noted previously that >20% of low cobalamin levels found in population surveys do not meet metabolic criteria of cobalamin deficiency,^{X} suggesting that at least some of them represent TC I deficiency.

### REFERENCES FOR EXAMPLE 3

A. Carmel R, Herbert V. Deficiency of vitamin B₁₂-binding alpha globulin in two brothers. Blood 1969; 33:1-12.
B. Carmel R. A new case of deficiency of the R binder for cobalamin, with observations on minor cobalamin-binding proteins in serum and saliva. Blood 1982; 59:152-156.
C. Carmel R. R binder deficiency: a clinically benign cause of cobalamin pseudo-deficiency. JAMA 1983; 250:1886-1890.
D. Jenks J, Begley J, Howard L. Cobalamin R binder deficiency in a woman with thalassemia. Nutr Rev 1983; 41:277-280.
E. Zittoun J, Léger J, Marquet J, Carmel R. Combined congenital deficiencies of intrinsic factor and R binder. Blood 1988; 72:940-943.
F. Hall C A, Begley J A. Congenital deficiency of human R-type binding proteins of cobalamin. Am J Hum Genet. 1977; 29, 619-626.
G. Carmel R. Cobalamin-binding proteins in man. In: Contemporary Hematology-Oncology. Silber R, Gordon A S, LoBue J, Muggia F M (eds.), Vol. 2, Plenum, New York. 1981: 79-129.
H. Carmel R. The distribution of endogenous cobalamin among cobalamin-binding proteins in the blood in normal and abnormal states. Am J Clin Nutr 1985; 41:713-719.
I. Carmel R. Mild transcobalamin I (haptocorrin) deficiency and low serum cobalamin concentrations. Clin Chem 2003; 49:1367-1374.
J. Carmel R, Parker J, Kelman Z. Genomic mutations associated with mild and severe deficiencies of transcobalamin I (haptocorrin) that cause mildly and severely low serum cobalamin levels. Br J Haematol 2009; 147:386-391; and Example 1.
K. Johnston J, Bollekens J, Allen R H, Berliner N. Structure and expression of the cDNA encoding transcobalamin I, a neutrophil granule protein. J Biol Chem 1989; 264:15754-15757.
   L. Johnston J, Yang-Feng T, Berliner N. Genomic structure and mapping of the chromosomal gene for transcobalamin I (TCN1): comparison to human intrinsic factor. Genomics 1992; 12:459-464.
M. Monsen A L, Refsum H, Larkestad T, Ueland P M. Cobalamin status and its biochemical markers methylmalonic acid and homocysteine in different age groups from 4 days to 19 years. Clin Chem 2003; 49:2067-2075.
N. Lin J C, Borregaard N, Liebman H A, Carmel R. Deficiency of the specific granule proteins, R binder/transcobalamin I and lactoferrin, in plasma and saliva: a new disorder. Am J Med Genet. 2001; 100:145-51.
O. Wuerges J, Garau G, Geremia S, Fedosov S N, Petersen T E, Randaccio L. Structural basis for mammalian vitamin B12 transport by transcobalamin. Proc Natl Acad Sci USA 2006; 103:4386-4391.
P. Wuerges J, Geremia S, Randaccio L. Structural study on ligand specificity of human vitamin B12 transporters. Biochem J 2007; 403:431-440.
Q. Hazra A, Kraft P, Selhub J, Giovannucci E L, Thomas G, Hoover R N, Chanock Hunter D J. Common variants of FUT2 are associated with plasma vitamin B12 levels. Nature Genet. 2008; 40:1160-1162.
R. Burger R L, Mehlman C S, Allen R H. Human plasma R-type vitamin B12-binding proteins. I. Isolation and characterization of transcobalamin I, transcobalamin III, and the normal granulocyte vitamin B12-binding protein. J Biol Chem 1975; 250:7700-7706.
S. Burger R L, Scheider R J, Mehlman C S, Allen R H. Human plasma R-type vitamin B12-binding proteins. II. The role of transcobalamin I, transcobalamin III, and the normal granulocyte vitamin B12-binidng protein in the transport of vitamin B12. J Biol Chem 1975; 250:7707-7713.
T. Nexo E, Olesen H, Chrystenssen J M, Thomsen J, Kristiansen K. Characterization of a cobalamin-binding plasma protein from a patient with hepatoma. Scand J Clin Lab Invets 1975; 35:683-690.
U. Boisson F, Fremont S, Migeon C, Nodari F, Droesch S, Gerard P, Parache R M, Nicolas J P. Human haptocorrin in hepatocellular carcinoma. Cancer Detect Prey 1999; 23:89-96.
V. Carmel R, Brar S, Frouhar Z. Plasma total transcobalamin I. Ethnic/racial patterns and comparison with lactoferrin. Am J Clin Pathol 2001; 116:576-580.
W. Single nucleotide polymorphisms. National Center for Biotechnology Information. US National Library of Medicine, Bethesda, Md. http://www.ncbi.nlm.nih.gov/SNP/snp_ref.cgi?rs=34324219. (Accessed May 25, 2009)
X. Carmel R. Current concepts in cobalamin deficiency. Annu Rev Med 2000; 51:357-375.
Y. Tanaka T, Scheet P, Giusti B, Bandinelli S, Piras M G, et al. Genome-wide association study of vitamin B6, vitamin B12, folic acid, and homocysteine blood concentrations. Am J Hum Genet. 2009; 84:477-482.

### Example 4

### Prophetic Example—Decreased Fucosylation of TC I by FUT2 Increases Serum Cobalamin Levels by Increasing TC I Levels, and May Counteract the Mild TC I Deficiency Caused by the 999G>T Mutation

Serum levels of cobalamin (vitamin B12), an essential vitamin, depend heavily on direct determinants such as intake and absorption of cobalamin. However, they are also subject to indirect, normutritional influences such as status of transcobalamin I (TC I), a binding protein that carries cobalamin in plasma, and other examples. As described in Examples 1 and 3 above, genetic influences can act on TC I via mutations of the TCN1 gene that create deficiency of TC I, which leads to low serum cobalamin. However, non-TCN1 genetic influences on TC I that lead to low or high cobalamin levels may also exist. In one possible example, FUT2 the gene for α,1,2-fucosyltransferase, may exert its effect on cobalamin via a genetic influence on TC I fucosylation that is not limited to secretions. The effects of FUT2 mutations may thereby also interact with the effects of TCN1 mutations in modulating the phenotype of TC I and cobalamin status, sometimes in one direction and sometimes in another.

TC I is the major carrier of cobalamin in the bloodstream, mainly because it does not deliver its cobalamin to tissues and is slowly cleared from plasma (half-life 9-10 days).¹ This contrasts with TC II and its cobalamin, which are both rapidly cleared to tissues in a matter of minutes.¹ Eventually, however, as TC I sheds sialic acid residues and undergoes other undetermined carbohydrate changes, it is cleared nonspecifically by hepatic asialoglycoprotein receptors and presumably takes its cobalamin along.² TC I is heavily glycosylated; 35-40% of its mass is carbohydrate.³ Fucose is a major moiety and is one of two carbohydrates that have varied most distinctly among the different isoforms of TC I, the other being sialic acid (FIG. 15).³⁻⁵ TC I isoforms with less fucose tend to have more sialic acid, and abnormal disease-related TC I isoforms have much less fucose than the various normal TC I isoforms from different sources (FIG. 15).

The relatively sialylated but fucose-depleted TC I isoforms are cleared especially slowly and accumulate in the blood, whereas the desialylated and normally fucosylated TC I isoforms are cleared more rapidly.^{2,3} As a result, the diseases associated with decreased fucose shown in FIG. 15, such as chronic myelogenous leukemia and hepatocellular cancer, tend to accumulate very high TC I levels in the plasma (e.g., >1000 to 10,000 pmol/l versus normal TC I levels of 165-450).^{1,5-8} They also accumulate very high serum cobalamin levels >1000 to 10,000 pmol. These changes are dramatic, and are conditions that are acquired rather than genetic (although epigenetic mechanisms may operate). Furthermore, such conditions may be affected by cancer-related processes as well.⁵⁻⁸ It has been shown that hepatocellular cancer tissue have lower FUT2 activity than noncancerous liver, whereas FUT6 activity and galactosidase activity were increased in the cancer tissue.⁹

Hazra et al.¹⁰ conducted a genome wide scan of samples from women enrolled in the Nurses Health Study, comparing mutation results with serum cobalamin levels. They identified 3 gene mutations of the FUT2 gene that either caused or were closely linked (e.g., by linkage dysequilibrium) or were proxies for mutations that caused significant differences in cobalamin levels. Thus, they reported significantly higher serum cobalamin levels in women who were homozygous for the rs492602 [G] allele (247A>G) than women who did not have the FUT2 mutation or were only heterozygous for it (mean cobalamin 497 pmol/l vs. 420 pmol/l). Tanaka et al¹¹ found similar trends, although the FUT2 mutation they linked most strongly with cobalamin was rs602662 [A], which Hazra et al¹⁰ showed as one of several closely linked mutations, leaving the question open as to which one or more (or others also linked but still undiscovered) polymorphisms may be associated with cobalamin elevation.

The mathematical cobalamin associations, although they have proven very useful, do not resolve the nature of the association or which FUT2 mutation holds the key. It is postulated that one or more known or unknown FUT2 mutants linked to the published single-nucleotide polymorphisms reduce TC I fucosylation, resulting in slower clearance of TC I and, thus mildly higher TC I levels and, as a result, mildly higher serum cobalamin levels.

As described in Example 3, the 999G>T mutation of TCN1, which causes a substitution of tyrosine for aspartic acid in amino acid 301, is associated with mildly low plasma TC I levels and, because of the low TC I, also with mildly low serum cobalamin levels. Four propositi with heterozygosity for the 999G>T mutation were identified who exhibited such phenotypes. The 4 relatives in the studied families who had similarly low TC I and cobalamin levels all showed the 999G>T mutation too. On the other hand, the 4 relatives who had normal TC I and cobalamin levels did not have the 999G>T mutation.

In contrast, the one exceptional control subject (out of 25 tested control subjects) with normal TC I (260-370 pmol/l) and cobalamin levels (300-370 pmol/l) was identified who was heterozygous for the 999G>T TCN1 mutation (24 other tested normal controls did not have this mutation). On further testing, this control subject was found to be homozygous for the FUT2 mutation(s) associated with higher cobalamin levels. In further contrast to him, none of the 4 patients and relatives in the two 999G>T-positive families who showed low plasma TC I and cobalamin levels were homozygous for the FUT2 mutation(s). All 4 had the wild type or were heterozygotes for the FUT2 mutation(s), neither of which genotypes are associated with the higher cobalamin levels.

Moreover, the mother and sister of the exceptional control subject had similarly normal plasma TC I and cobalamin levels as he did (data not shown). Similarly, both of them were heterozygotes for the 999G>T TC I mutation that should cause TC I deficiency and homozygotes for the FUT2 mutations. This identical findings in all 3 family members supports the genetic explanations for the paradox.

The findings suggest that when homozygosity for the FUT2 mutation(s) coexists with the 999G>T TCN1 mutation, it may nullify the decline in plasma TC I (and secondarily, in cobalamin levels as well) caused by the 999G>T mutation and its amino acid substitution. Whatever structural defect results from the TC I amino acid substitution, its lowering of TC I levels may be counteracted by the slow plasma TC I clearance associated with the reduced fucosylation of the TC I protein by the defective fucosyltransferase caused by homozygosity for the FUT2 mutation(s).

It is therefore proposed that FUT2 mutation produces less severe fucosylation changes than seen in acquired conditions such as hepatocellular cancer or chronic myelogenous leukemia, with their massive TC I and cobalamin elevations described previously and shown in FIG. 15. A milder fucosylation change than the large acquired changes of the disease examples would explain the relatively mild increase in TC I and cobalamin levels the FUT2 mutation(s) caused in the patient described above that created normal serum TC I and cobalamin levels despite heterozygosity for the 999G>T TCN1 mutation, and in the study by Ham et al¹⁰.

### REFERENCES FOR EXAMPLE 4

1. Carmel R. Cobalamin-binding proteins in man. In: Contemporary Hematology-Oncology. Silber R, Gordon A S, LoBue J, Muggia F M (eds.), Vol. 2, Plenum, New York. 1981: 79-129.
2. Burger R L, Schneider R J, Mehlman C S, Allen R H. Human plasma R-type vitamin B12-binding proteins. II. The role of transcobalamin I, transcobalamin III, and the normal granulocyte vitamin B12-binidng protein in the transport of vitamin B12. J Biol Chem 1975; 250:7707-7713.
3. Burger R L, Waxman S, Gilbert H S, Mehlman C S, Allen R H. Isolation and characterization of a novel vitamin B12-binding protein associated with hepatocellular carcinoma. J Clin Invest 1975; 56:1262-1270.
4. Burger R L, Mehlman C S, Allen R H. Human plasma R-type vitamin B12-binding proteins. I. Isolation and characterization of transcobalamin I, transcobalamin III, and the normal granulocyte vitamin B12-binding protein. J Biol Chem 1975; 250:7700-7706.
5. Nexo E, Olesen H, Chrystenssen J M, Thomsen J, Kristiansen K. Characterization of a cobalamin-binding plasma protein from a patient with hepatoma. Scand J Clin Lab Invest 1975; 35:683-690.
6. Waxman S, Gilbert H C. Characteristics of a novel serum vitamin B12-binding protein associated with hepatocellular carcinoma. R J Haematol 1974; 27:229-239.
7. Waxman 5, Liu C K, Schreiber C, Helso L. The clinical and physiological implications of hepatoma B12-binding proteins. Cancer Res 1977; 37:1908-1914.
8. Boisson F, Fremont S, Migeon C, Nodari F, Droesch S, Gerard P, Parache R M, Nicolas J P. Human haptocorrin in hepatocellular carcinoma. Cancer Detect Prey 1999; 23:89-96.
9. Hutchinson W L, Du M Q, Johnson P J, Williams R. Fucsyltransferases: differential plasma and tissue alterations in hepatocellular carcinoma and cirrhosis. Hepatol 1992; 13:683-688.
10. Hazra A, Kraft P, Selhub J, Giovannucci E L, Thomas G, Hoover R N, Chanock S J, Hunter D J. Common variants of FUT2 are associated with plasma vitamin B12 levels. Nature Genet. 2008; 40:1160-1162.
11. Tanaka T, Scheet P, Giusti B, Bandinelli S, Piras M G, et al. Genome-wide association study of vitamin B6, vitamin B12, folic acid, and homocysteine blood concentrations. Am J Hum Genet. 2009; 84:477-482.

### Example 5

### Double Heterozygosity for Two Mutations of TCN1 Associated with the Severe TC I Deficiency Phenotype in a Black Patient

At the age of 83 years, transcobalamin I (TC I) deficiency was diagnosed in a 77 year-old African-American man who had received cobalamin injections and oral folic acid continuously since a low serum cobalamin level (and folate level) was detected >4 years previously. The cobalamin level, reported as <50 ng/l, had been measured because of chronic anemia: hemoglobin of 10.3 g/dl, MCV 92 fl, WBC count 3200/μl, and mild neutrophil hypersegmentation were noted. H is serum folate level was 3 μg/l, and there was a history of alcohol abuse. There was little evidence of hematologic improvement after cobalamin and folate therapy other than correction of the neutrophil hypersegmentation; MCV continued to fluctuate between 85 and 97 fl in the intervening years despite continued cobalamin injections and was attributed, along with persistent, mild vibratory sense loss in the toes and the original folate deficiency, to alcohol abuse. Low serum folate levels recurred several times as alcohol abuse persisted. The cobalamin level stayed at normal levels after cobalamin therapy was initiated. Besides his alcohol abuse, he had gout, mild renal insufficiency, substernal thyroid goiter, an ocular cataract, false positive serology for syphilis, and Forrestier's disease.

Evaluation for his originally low cobalamin was delayed for several years, in part because of his reluctance. A borderline low-normal Schilling test result (7.8% excretion; normal >8.0%) appeared to be attributable to mild renal insufficiency, as pernicious anemia was ruled out by normal gastric acid secretion upon gastric analysis and intrinsic factor antibody was undetectable. There was evidence of Helicobacter pylori infection with gastritis. Plasma and saliva TC I levels were consistently virtually undetectable (radioimmunoassay showed a plasma TCI of 12 pmol/l, rather than zero, however; normal=165-454) and his plasma cobalamin was shown to be bound almost entirely to TC II and a minor 70,000-molecular weight peak on chromatography instead of recognizable TC I¹. Hemoglobin electrophoresis revealed sickle cell trait, as found in other patients with severe TC I deficiency (the patient is identified by reference O and shown in FIG. 9). The patient died with congestive heart failure at another hospital at the age of 84 years.

His case demonstrates that TC I deficiency can be first diagnosed in very old age and can mimic subclinical cobalamin deficiency, which is common at that age. It also demonstrates that small amount of minor unidentified cobalamin-binding proteins can be detected by chromatographic fractionation when deficiency is severe and the minor peak is not obscured by TC I.

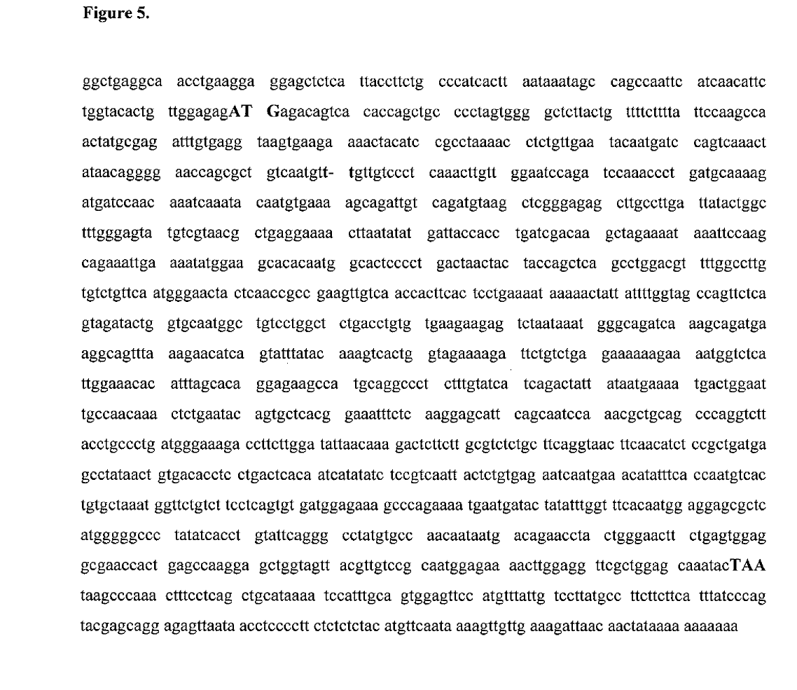

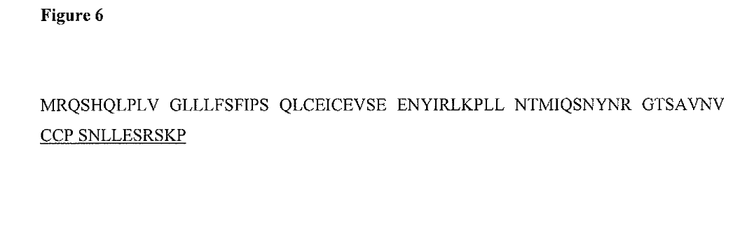

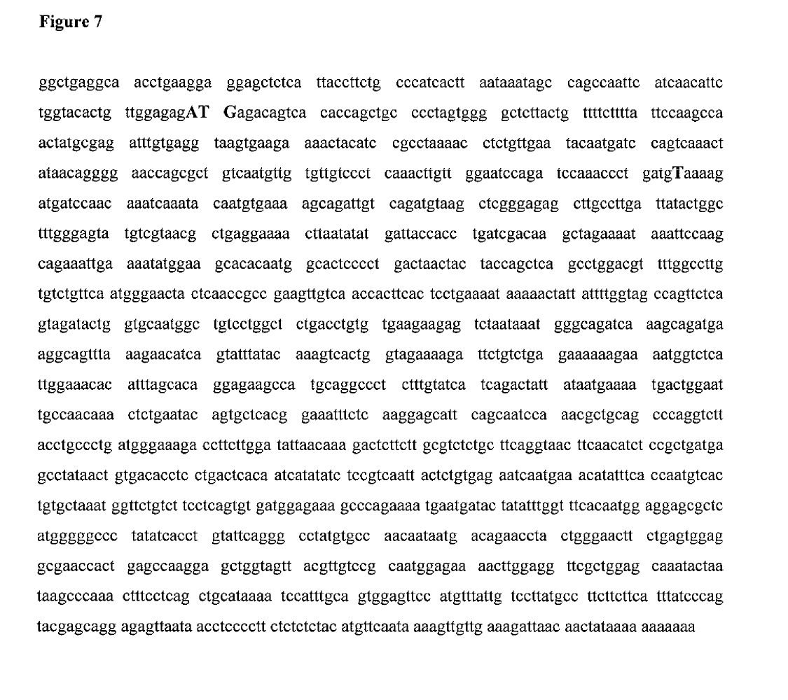

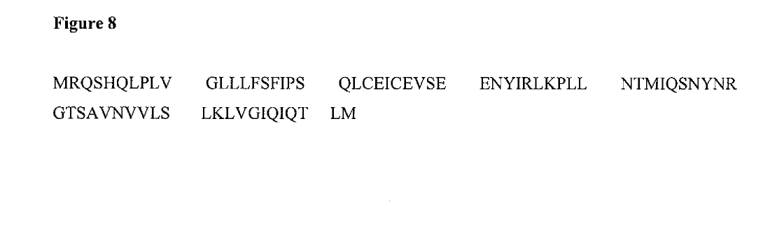

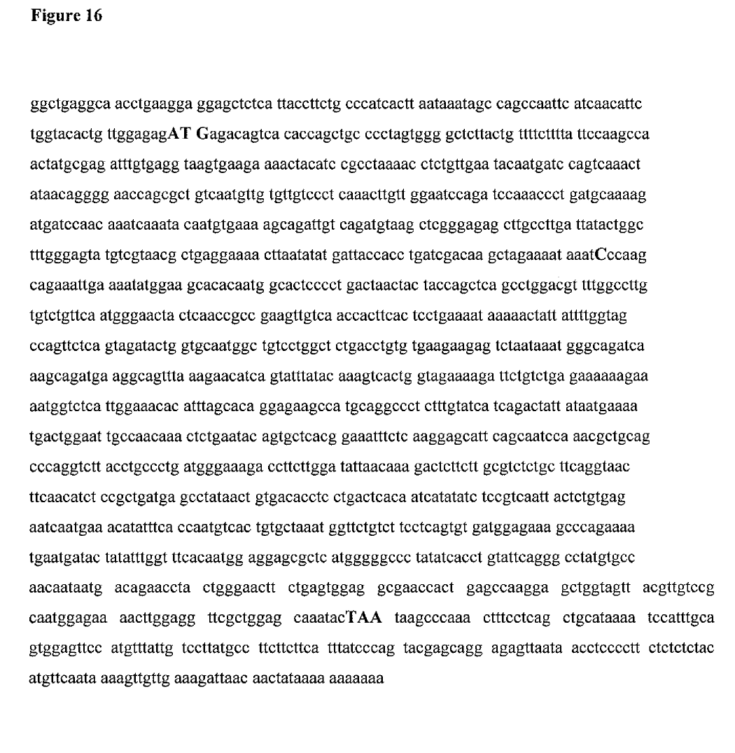

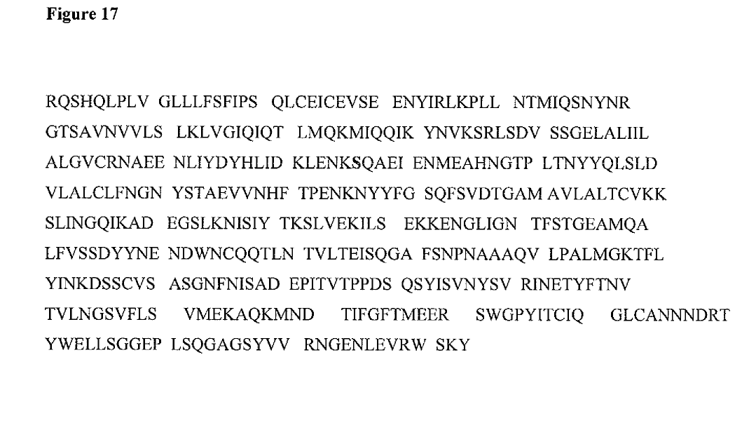

DNA was extracted from an archived serum sample many years after his death. He was found to be heterozygous for the nonsense 270delG mutation that was described in other black patients with TC I deficiency in this application (FIGS. 5 and 6). Search of the other 8 exons revealed additional heterozygosity for a new additional missense mutation, 475T>C (FIGS. 15 and 16). This mutation substitutes the smaller, polar serine for a larger, aromatic phenylalanine, but its effects cannot be precisely predicted because the structure of the heavily glycosylated protein, TC I, has not been determined. Perhaps the heterozygous amino acid substitution, by not also promoting nonsense mediated degradation that the nonsense mutation does, leaves the tiny amount of altered, perhaps only partially immunoreactive TC I in plasma and secretions seen in the patient's plasma.

### REFERENCES FOR EXAMPLE 5

1. Carmel R. A new case of deficiency of the R binder for cobalamin, with observations on minor cobalamin-binding proteins in serum and saliva. Blood 1982; 59:152-1156.

TABLE 1
Listing of Sequences in Humans

| SEQ ID NO. | Sequence |
|---|---|
| SEQ ID NO: 1 | human transcobalamin I gene (TCN1) |
| | (GenBank Accession No. NP_001053) |
| SEQ ID NO: 2 | human transcobalamin I amino acid |
| | sequence (GenBank Accession No. |
| | NP_001053) |
| SEQ ID NO: 3 | deletion of nucleotide 270 of human |
| | TCN1 |
| SEQ ID NO: 4 | CCPSNLLESRSKP |
| SEQ ID NO: 5 | 70 amino acid polypeptide expressed |
| | by a human mutant TCN1 comprising a |
| | deletion of nucleotide 270. |
| SEQ ID NO: 6 | C > T nonsense point mutation at |
| | nucleotide 315 of human TCN1 |
| SEQ ID NO: 7 | 72 amino acid polypeptide expressed |
| | by a human mutant TCN1 comprising a |
| | C > T nonsense point mutation at |
| | nucleotide 315. |
| SEQ ID NO: 8 | CCCGAAGGTTTAGGACAGGAGAC |
| SEQ ID NO: 9 | GGAGTCCAACCACATACGAAACTGG |
| SEQ ID NO: 10 | CCACCAACACAGTCTGCAGCCACTGGATTGG |
| SEQ ID NO: 11 | CCATTAGAAGGGATGTAGCAGGGATACTTTGG |
| SEQ ID NO: 12 | GGCAATATAAGTCTCAAGGAAATCAGGAGGCCC |
| SEQ ID NO: 13 | GTGCCCTCAAAGACATAGTGAGATGAACGGG |
| SEQ ID NO: 14 | GGGTGACCTCCCCCTTCTATTAGCCACCTTTCC |
| SEQ ID NO: 15 | GAGGGGACTAGAGCAAAGAGGGTAG |
| SEQ ID NO: 16 | CTCACATCCCAGGAAACCTCTCGGCCCAGG |
| SEQ ID NO: 17 | CCAGCCTGGGCAACAAGAGCGAAGCTCCATC |
| SEQ ID NO: 18 | GATAACTGAGTTATCTGAGGTGCTTCCTAGG |
| SEQ ID NO: 19 | CCTCAGGTGACTGTAAACCCTGGTAACATGAGG |
| SEQ ID NO: 20 | GGCAAGGAGTCCTTGGATAGGGGTTGAGTAGG |
| SEQ ID NO: 21 | GGACAGCAAAGCTACTGACCCAGAAGC |
| SEQ ID NO: 22 | GCATGCAGATTCCTGATCCCCAGCTC |
| SEQ ID NO: 23 | GCCCAATCCCCACAGAGGCTCACACCCC |
| SEQ ID NO: 24 | CTTCTGAGTGGAGGCGAACCACTGAGCC |
| SEQ ID NO: 25 | AGTTAGTAGGAGAACCTTGAGTAGAACCCC |
| SEQ ID NO: 26 | G > T missense point mutation at |
| | nucleotide 999 of human TCN1 |
| SEQ ID NO: 27 | 433 amino acid polypeptide expressed |
| | by a human mutant TCN1 comprising a |
| | G > T missense point mutation at |
| | nucleotide 999 of human TCN1. |
| SEQ ID NO: 28 | T > C missense point mutation at |
| | nucleotide 475 of human TCN1 |
| SEQ ID NO: 29 | 433 amino acid polypeptide expressed |
| | by a human mutant TCN1 comprising a |
| | T > C missense point mutation at |
| | nucleotide 475 of human TCN1. |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, GenBank Accession Numbers, and protocols are cited throughout this application, the disclosures of which are incorporated herein by reference in their entireties for all purpose.

1. A method for detecting transcobalamin I (TC I) deficiency in a human or other mammal comprising: detecting at least one TCN1 mutation in a sample from the mammal, wherein detecting the TCN1 mutation indicates hereditary TC I deficiency.

2. The method of claim 1, wherein at least one TCN1 mutation is selected from the group consisting of a deletion at nucleotide 270 of human TCN1, a C>T nonsense point mutation at nucleotide 315 of human TCN1, a G>T missense point mutation at nucleotide 999 of human TCN1, and a T>C missense point mutation of nucleotide 475 of human TCN1, wherein TCN1 comprises the nucleic acid sequence defined by SEQ ID NO:1.

3. The method of claim 2, wherein at least one TCN1 mutation is a deletion at nucleotide 270 of human TCN1.

4. The method of claim 2, wherein at least one TCN1 mutation is a C>T nonsense point mutation at nucleotide 315 of human.

5. The method of claim 2, wherein at least one TCN1 mutation is a G>T missense point mutation at nucleotide 999 of human TCN1.

6. The method of claim 2, wherein at least one TCN1 mutation is T>C missense point mutation of nucleotide 475 of human TCN1.

7. The method of claim 2, wherein homozygosity or compound heterozygosity of the TCN1 mutation produces a severe transcobalamin I deficiency.

8. The method of claim 2, wherein a TCN1 mutation in a single TCN1 allele produces a mild transcobalamin I deficiency.

9. A method for detecting transcobalamin I (TC I) deficiency in a human or other mammal comprising: detecting at least one TC I isoform in a sample from the mammal, wherein detecting the TC I isoform indicates TC I deficiency, and further wherein the TC I isoform is a polypeptide selected from the group consisting of SEQ ID NO:5, SEQ ID NO:7, SEQ ID NO:27 and SEQ ID NO: 29.

10. The method of claim 1, wherein the sample is selected from the group consisting of plasma, serum, cerebrospinal fluid, sputum, saliva, breast milk, tears, bile, semen, vaginal secretion, amniotic fluid, urine, stool, leukocytes, bone marrow cells, buccal cells, fibroblasts and tissue biopsies.

11. The method of claim 1, wherein the detecting is selected from nucleic acid detection and immuno detection.

12. The method of claim 11, wherein the detecting is nucleic acid detection and the nucleic acid is genomic DNA or RNA.

13. The method of claim 11, wherein the detecting is nucleic acid detection and the nucleic acid detection is an assay selected from the group consisting of polymerase chain reaction (PCR), quantitative PCR, nucleic acid sequencing, and nucleic acid microarray analysis.

14. The method of claim 11, wherein the detecting is immunodetection and the immunodetection is selected from the group consisting of ELISA, Western blot, and radioimmunoassay (RIA), and wherein the TC I isoform comprises a TC I protein fragment.

15. The method of claim 11, wherein the detecting is immunodetection and immunologic detection comprises detection with an antibody selected from the group consisting of a polyclonal antibody and a monoclonal antibody.

16. The method of claim 11, wherein the detecting is immunodetection and the immunodetection is ELISA, and further wherein the ELISA is a sandwich ELISA comprising contacting the TC I isoform with a first antibody preparation fixed to a substrate and a second antibody preparation labeled with an enzyme.

17. The method of claim 16, wherein the first antibody preparation is monoclonal.

18. The method of claim 16, wherein the second antibody preparation is polyclonal.

19. A method of detecting a mutation in a TCN1 gene in a sample, the method comprising:
(a) amplifying at least one exon of the TCN1 gene or fragments thereof;
(b) detecting the amplified nucleic acid portions; and
(c) comparing the amplified nucleic acid to a non-mutant TCN1 nucleic acid sequence,
wherein the non-mutant TCN1 nucleic acid sequence is defined by SEQ ID NO:1, and wherein a difference between the sequence of the amplified nucleic acid sequence and SEQ ID NO:1 indicates the presence of a mutation in the sample.

20. The method of claim 19, wherein the sample is DNA derived from tissues, cells and/or cells in biological fluids.

21. The method of claim 19, wherein the tissues, cells and/or cells in biological fluids are derived from human patients.

22. The method of claim 21, wherein the biological fluids are selected from a group of biological fluids consisting of plasma, serum, cerebrospinal fluid, sputum, breast milk, saliva, tears, bile, semen, vaginal secretion, amniotic fluid, urine or stool.

23. The method of claim 19, wherein oligonucleotide primer pairs are utilized to amplify at least one exon of the TCN1 gene or portion thereof, and wherein the primer pairs are selected from the group consisting of SEQ ID NO:8 and 9, SEQ ID NO:10 and 11, SEQ ID NO:12 and 13, SEQ ID NO:14 and 15, SEQ ID NO:16 and 17, SEQ ID NO:18 and 19, SEQ ID NO:20 and 21, SEQ ID NO:22 and 23, and SEQ ID NO:24 and 25.

24. The method of claim 19, wherein the method utilizes quantitative PCR techniques to determine relative quantitative levels of a TCN1 mutation.

25-35. (canceled)

36. A method of determining whether an individual with a low serum cobalamin level suffers from true cobalamin deficiency, comprising determining whether the individual carries at least one TCN1 gene mutation and/or expresses at least one TC I protein isoform, wherein detecting the TCN1 mutation or detecting the TC I isoform indicates a TC I deficiency and not a cobalamin deficiency.

37. The method of claim 36, wherein the individual with a low serum cobalamin level and no TCN1 gene mutation or no TC I protein isoform is a valid candidate for cobalamin therapy and treatment is initiated.

38. A method for treating an individual with a low serum cobalamin level, comprising:
(a) testing a sample from the individual for the presence of at least one TCN1 gene mutation and/or at least one TC I protein isoform; and
(b) administering cobalamin therapy to the subject if at least one TCN1 gene mutation and/or TC I protein isoform is not present; and not administering cobalamin therapy to the individual if at least one TCN1 gene mutation and/or TC I protein isoform is present.

39. The method of claim 38, wherein at least one TCN1 mutation is detected in a human, and wherein the mutation is also associated with a second disorder.

40. The method of claim 39, wherein the second disorder is sickle cell anemia or sickle cell trait.

41. The method of claim 40, wherein the second disorder is sickle cell anemia or sickle cell trait, and wherein at least one TCN1 mutation is selected from the group consisting of a deletion at nucleotide 270 of human TCN1, a C>T nonsense point mutation at nucleotide 315 of human TCN1, or a T>C missense point mutation of nucleotide 475 of human TCN1.

42. A method for increasing the level of TC I in an individual comprising the step of inhibiting TC I fucosylation, wherein inhibiting TC I fucosylation decreases the rate of TC I clearance from the individual's blood.

43. The method of claim 42, further comprising the step of inhibiting the expression of α,1,2-fucosyltransferase (FUT2) in the individual.

44. The method of claim 42, wherein the expression of α,1,2-fucosyltransferase (FUT2) is inhibited by administering FUT2 antisense, FUT2 RNAi, FUT2 antibodies, or small molecule inhibitors of FUT2 to the individual.

45. The method of claim 42, wherein inhibiting TC I fucosylation increases the level of serum cobalamin in the individual.

46. A method for decreasing the level of TC I in an individual comprising the step of increasing TC I fucosylation, wherein increasing TC I fucosylation increases the rate of TC I clearance from the individual's blood.

47. The method of claim 46, further comprising the step of increasing the expression of α,1,2-fucosyltransferase (FUT2) in the individual.

48. The method of claim 47, wherein the expression of α,1,2-fucosyltransferase is increased by administering to the individual a FUT2 polypeptide or an expression vector comprising a FUT2 gene operably linked to a promoter.

49. The method of claim 46, wherein increasing TC I fucosylation decreases the level of serum cobalamin in the individual.

50. The method of claim 42, wherein the individual has a G>T missense point mutation at nucleotide 999 of a TCN1 gene, and is homozygous for a FUT2 mutation that reduces fucosyltransferase activity, wherein the TCN1 and FUT2 mutations at least partially functionally neutralize the other's effect on TC I and cobalamin levels in the individual.

51. The method of claim 42, wherein TC I fucosylation is inhibited in plasma, blood cells, leukocytes or bone marrow leukocytic precursors, where TC I is synthesized.

52-55. (canceled)


SEQUENCE LISTING (29 sequences)

SEQ ID NO: 1 - DNA, 1577 bp, Homo sapiens
ggctgaggcaacctgaaggaggagctctcattaccttctgcccatcacttaataaatagccagccaattcatcaacattctggtacactgttggagagatgagacagtcacaccagctgcccctagtggggctcttactgttttcttttattccaagccaactatgcgagatttgtgaggtaagtgaagaaaactacatccgcctaaaacctctgttgaatacaatgatccagtcaaactataacaggggaaccagcgctgtcaatgttgtgttgtccctcaaacttgttggaatccagatccaaaccctgatgcaaaagatgatccaacaaatcaaatacaatgtgaaaagcagattgtcagatgtaagctcgggagagcttgccttgattatactggctttgggagtatgtcgtaacgctgaggaaaacttaatatatgattaccacctgatcgacaagctagaaaataaattccaagcagaaattgaaaatatggaagcacacaatggcactcccctgactaactactaccagctcagcctggacgttttggccttgtgtctgttcaatgggaactactcaaccgccgaagttgtcaaccacttcactcctgaaaataaaaactattattttggtagccagttctcagtagatactggtgcaatggctgtcctggctctgacctgtgtgaagaagagtctaataaatgggcagatcaaagcagatgaaggcagtttaaagaacatcagtatttatacaaagtcactggtagaaaagattctgtctgagaaaaaagaaaatggtctcattggaaacacatttagcacaggagaagccatgcaggccctctttgtatcatcagactattataatgaaaatgactggaattgccaacaaactctgaatacagtgctcacggaaatttctcaaggagcattcagcaatccaaacgctgcagcccaggtcttacctgccctgatgggaaagaccttcttggatattaacaaagactcttcttgcgtctctgcttcaggtaacttcaacatctccgctgatgagcctataactgtgacacctcctgactcacaatcatatatctccgtcaattactctgtgagaatcaatgaaacatatttcaccaatgtcactgtgctaaatggttctgtcttcctcagtgtgatggagaaagcccagaaaatgaatgatactatatttggtttcacaatggaggagcgctcatgggggccctatatcacctgtattcagggcctatgtgccaacaataatgacagaacctactgggaacttctgagtggaggcgaaccactgagccaaggagctggtagttacgttgtccgcaatggagaaaacttggaggttcgctggagcaaatactaataagcccaaactttcctcagctgcataaaatccatttgcagtggagttccatgtttattgtccttatgccttcttcttcatttatcccagtacgagcaggagagttaataacctccccttctctctctacatgttcaataaaagttgttgaaagattaacaactataaaaaaaaaaa

SEQ ID NO: 2 - protein, 433 aa, Homo sapiens
MRQSHQLPLVGLLLFSFIPSQLCEICEVSEENYIRLKPLLNTMIQSNYNRGTSAVNVVLSLKLVGIQIQTLMQKMIQQIKYNVKSRLSDVSSGELALIILALGVCRNAEENLIYDYHLIDKLENKFQAEIENMEAHNGTPLTNYYQLSLDVLALCLFNGNYSTAEVVNHFTPENKNYYFGSQFSVDTGAMAVLALTCVKKSLINGQIKADEGSLKNISIYTKSLVEKILSEKKENGLIGNTFSTGEAMQALFVSSDYYNENDWNCQQTLNTVLTEISQGAFSNPNAAAQVLPALMGKTFLDINKDSSCVSASGNFNISADEPITVTPPDSQSYISVNYSVRINETYFTNVTVLNGSVFLSVMEKAQKMNDTIFGFTMEERSWGPYITCIQGLCANNNDRTYWELLSGGEPLSQGAGSYVVRNGENLEVRWSKY

SEQ ID NO: 3 - DNA, 1576 bp, Homo sapiens
ggctgaggcaacctgaaggaggagctctcattaccttctgcccatcacttaataaatagccagccaattcatcaacattctggtacactgttggagagatgagacagtcacaccagctgcccctagtggggctcttactgttttcttttattccaagccaactatgcgagatttgtgaggtaagtgaagaaaactacatccgcctaaaacctctgttgaatacaatgatccagtcaaactataacaggggaaccagcgctgtcaatgtttgttgtccctcaaacttgttggaatccagatccaaaccctgatgcaaaagatgatccaacaaatcaaatacaatgtgaaaagcagattgtcagatgtaagctcgggagagcttgccttgattatactggctttgggagtatgtcgtaacgctgaggaaaacttaatatatgattaccacctgatcgacaagctagaaaataaattccaagcagaaattgaaaatatggaagcacacaatggcactcccctgactaactactaccagctcagcctggacgttttggccttgtgtctgttcaatgggaactactcaaccgccgaagttgtcaaccacttcactcctgaaaataaaaactattattttggtagccagttctcagtagatactggtgcaatggctgtcctggctctgacctgtgtgaagaagagtctaataaatgggcagatcaaagcagatgaaggcagtttaaagaacatcagtatttatacaaagtcactggtagaaaagattctgtctgagaaaaaagaaaatggtctcattggaaacacatttagcacaggagaagccatgcaggccctctttgtatcatcagactattataatgaaaatgactggaattgccaacaaactctgaatacagtgctcacggaaatttctcaaggagcattcagcaatccaaacgctgcagcccaggtcttacctgccctgatgggaaagaccttcttggatattaacaaagactcttcttgcgtctctgcttcaggtaacttcaacatctccgctgatgagcctataactgtgacacctcctgactcacaatcatatatctccgtcaattactctgtgagaatcaatgaaacatatttcaccaatgtcactgtgctaaatggttctgtcttcctcagtgtgatggagaaagcccagaaaatgaatgatactatatttggtttcacaatggaggagcgctcatgggggccctatatcacctgtattcagggcctatgtgccaacaataatgacagaacctactgggaacttctgagtggaggcgaaccactgagccaaggagctggtagttacgttgtccgcaatggagaaaacttggaggttcgctggagcaaatactaataagcccaaactttcctcagctgcataaaatccatttgcagtggagttccatgtttattgtccttatgccttcttcttcatttatcccagtacgagcaggagagttaataacctccccttctctctctacatgttcaataaaagttgttgaaagattaacaactataaaaaaaaaaa

SEQ ID NO: 4 - protein, 13 aa, Artificial sequence
  Synthetic oligonucleotide
CCPSNLLESRSKP

SEQ ID NO: 5 - protein, 70 aa, Homo sapiens
MRQSHQLPLVGLLLFSFIPSQLCEICEVSEENYIRLKPLLNTMIQSNYNRGTSAVNVCCPSNLLESRSKP

SEQ ID NO: 6 - DNA, 1577 bp, Homo sapiens
ggctgaggcaacctgaaggaggagctctcattaccttctgcccatcacttaataaatagccagccaattcatcaacattctggtacactgttggagagatgagacagtcacaccagctgcccctagtggggctcttactgttttcttttattccaagccaactatgcgagatttgtgaggtaagtgaagaaaactacatccgcctaaaacctctgttgaatacaatgatccagtcaaactataacaggggaaccagcgctgtcaatgttgtgttgtccctcaaacttgttggaatccagatccaaaccctgatgtaaaagatgatccaacaaatcaaatacaatgtgaaaagcagattgtcagatgtaagctcgggagagcttgccttgattatactggctttgggagtatgtcgtaacgctgaggaaaacttaatatatgattaccacctgatcgacaagctagaaaataaattccaagcagaaattgaaaatatggaagcacacaatggcactcccctgactaactactaccagctcagcctggacgttttggccttgtgtctgttcaatgggaactactcaaccgccgaagttgtcaaccacttcactcctgaaaataaaaactattattttggtagccagttctcagtagatactggtgcaatggctgtcctggctctgacctgtgtgaagaagagtctaataaatgggcagatcaaagcagatgaaggcagtttaaagaacatcagtatttatacaaagtcactggtagaaaagattctgtctgagaaaaaagaaaatggtctcattggaaacacatttagcacaggagaagccatgcaggccctctttgtatcatcagactattataatgaaaatgactggaattgccaacaaactctgaatacagtgctcacggaaatttctcaaggagcattcagcaatccaaacgctgcagcccaggtcttacctgccctgatgggaaagaccttcttggatattaacaaagactcttcttgcgtctctgcttcaggtaacttcaacatctccgctgatgagcctataactgtgacacctcctgactcacaatcatatatctccgtcaattactctgtgagaatcaatgaaacatatttcaccaatgtcactgtgctaaatggttctgtcttcctcagtgtgatggagaaagcccagaaaatgaatgatactatatttggtttcacaatggaggagcgctcatgggggccctatatcacctgtattcagggcctatgtgccaacaataatgacagaacctactgggaacttctgagtggaggcgaaccactgagccaaggagctggtagttacgttgtccgcaatggagaaaacttggaggttcgctggagcaaatactaataagcccaaactttcctcagctgcataaaatccatttgcagtggagttccatgtttattgtccttatgccttcttcttcatttatcccagtacgagcaggagagttaataacctccccttctctctctacatgttcaataaaagttgttgaaagattaacaactataaaaaaaaaaa

SEQ ID NO: 7 - protein, 72 aa, Homo sapiens
MRQSHQLPLVGLLLFSFIPSQLCEICEVSEENYIRLKPLLNTMIQSNYNRGTSAVNVVLSLKLVGIQIQTLM

SEQ ID NO: 8 - DNA, 23 bp, Artificial sequence
  Synthetic oligonucleotide
cccgaaggtttaggacaggagac

SEQ ID NO: 9 - DNA, 25 bp, Artificial sequence
  Synthetic oligonucleotide
ggagtccaaccacatacgaaactgg

SEQ ID NO: 10 - DNA, 31 bp, Artificial sequence
  Synthetic oligonucleotide
ccaccaacacagtctgcagccactggattgg

SEQ ID NO: 11 - DNA, 32 bp, Artificial sequence
  Synthetic oligonucleotide
ccattagaagggatgtagcagggatactttgg

SEQ ID NO: 12 - DNA, 33 bp, Artificial sequence
  Synthetic oligonucleotide
ggcaatataagtctcaaggaaatcaggaggccc

SEQ ID NO: 13 - DNA, 31 bp, Artificial sequence
  Synthetic oligonucleotide
gtgccctcaaagacatagtgagatgaacggg

SEQ ID NO: 14 - DNA, 33 bp, Artificial sequence
  Synthetic oligonucleotide
gggtgacctcccccttctattagccacctttcc

SEQ ID NO: 15 - DNA, 25 bp, Artificial sequence
  Synthetic oligonucleotide
gaggggactagagcaaagagggtag

SEQ ID NO: 16 - DNA, 30 bp, Artificial sequence
  Synthetic oligonucleotide
ctcacatcccaggaaacctctcggcccagg

SEQ ID NO: 17 - DNA, 31 bp, Artificial sequence
  Synthetic oligonucleotide
ccagcctgggcaacaagagcgaagctccatc

SEQ ID NO: 18 - DNA, 31 bp, Artificial sequence
  Synthetic oligonucleotide
gataactgagttatctgaggtgcttcctagg

SEQ ID NO: 19 - DNA, 33 bp, Artificial sequence
  Synthetic oligonucleotide
cctcaggtgactgtaaaccctggtaacatgagg

SEQ ID NO: 20 - DNA, 32 bp, Artificial sequence
  Synthetic oligonucleotide
ggcaaggagtccttggataggggttgagtagg

SEQ ID NO: 21 - DNA, 27 bp, Artificial sequence
  Synthetic oligonucleotide
ggacagcaaagctactgacccagaagc

SEQ ID NO: 22 - DNA, 26 bp, Artificial sequence
  Synthetic oligonucleotide
gcatgcagattcctgatccccagctc

SEQ ID NO: 23 - DNA, 28 bp, Artificial sequence
  Synthetic oligonucleotide
gcccaatccccacagaggctcacacccc

SEQ ID NO: 24 - DNA, 28 bp, Artificial sequence
  Synthetic oligonucleotide
cttctgagtggaggcgaaccactgagcc

SEQ ID NO: 25 - DNA, 30 bp, Artificial sequence
  Synthetic oligonucleotide
agttagtaggagaaccttgagtagaacccc

SEQ ID NO: 26 - DNA, 1577 bp, Homo sapiens
ggctgaggcaacctgaaggaggagctctcattaccttctgcccatcacttaataaatagccagccaattcatcaacattctggtacactgttggagagatgagacagtcacaccagctgcccctagtggggctcttactgttttcttttattccaagccaactatgcgagatttgtgaggtaagtgaagaaaactacatccgcctaaaacctctgttgaatacaatgatccagtcaaactataacaggggaaccagcgctgtcaatgttgtgttgtccctcaaacttgttggaatccagatccaaaccctgatgcaaaagatgatccaacaaatcaaatacaatgtgaaaagcagattgtcagatgtaagctcgggagagcttgccttgattatactggctttgggagtatgtcgtaacgctgaggaaaacttaatatatgattaccacctgatcgacaagctagaaaataaattccaagcagaaattgaaaatatggaagcacacaatggcactcccctgactaactactaccagctcagcctggacgttttggccttgtgtctgttcaatgggaactactcaaccgccgaagttgtcaaccacttcactcctgaaaataaaaactattattttggtagccagttctcagtagatactggtgcaatggctgtcctggctctgacctgtgtgaagaagagtctaataaatgggcagatcaaagcagatgaaggcagtttaaagaacatcagtatttatacaaagtcactggtagaaaagattctgtctgagaaaaaagaaaatggtctcattggaaacacatttagcacaggagaagccatgcaggccctctttgtatcatcagactattataatgaaaatgactggaattgccaacaaactctgaatacagtgctcacggaaatttctcaaggagcattcagcaatccaaacgctgcagcccaggtcttacctgccctgatgggaaagaccttcttgtatattaacaaagactcttcttgcgtctctgcttcaggtaacttcaacatctccgctgatgagcctataactgtgacacctcctgactcacaatcatatatctccgtcaattactctgtgagaatcaatgaaacatatttcaccaatgtcactgtgctaaatggttctgtcttcctcagtgtgatggagaaagcccagaaaatgaatgatactatatttggtttcacaatggaggagcgctcatgggggccctatatcacctgtattcagggcctatgtgccaacaataatgacagaacctactgggaacttctgagtggaggcgaaccactgagccaaggagctggtagttacgttgtccgcaatggagaaaacttggaggttcgctggagcaaatactaataagcccaaactttcctcagctgcataaaatccatttgcagtggagttccatgtttattgtccttatgccttcttcttcatttatcccagtacgagcaggagagttaataacctccccttctctctctacatgttcaataaaagttgttgaaagattaacaactataaaaaaaaaaa

SEQ ID NO: 27 - protein, 433 aa, Homo sapiens
MRQSHQLPLVGLLLFSFIPSQLCEICEVSEENYIRLKPLLNTMIQSNYNRGTSAVNVVLSLKLVGIQIQTLMQKMIQQIKYNVKSRLSDVSSGELALIILALGVCRNAEENLIYDYHLIDKLENKFQAEIENMEAHNGTPLTNYYQLSLDVLALCLFNGNYSTAEVVNHFTPENKNYYFGSQFSVDTGAMAVLALTCVKKSLINGQIKADEGSLKNISIYTKSLVEKILSEKKENGLIGNTFSTGEAMQALFVSSDYYNENDWNCQQTLNTVLTEISQGAFSNPNAAAQVLPALMGKTFLYINKDSSCVSASGNFNISADEPITVTPPDSQSYISVNYSVRINETYFTNVTVLNGSVFLSVMEKAQKMNDTIFGFTMEERSWGPYITCIQGLCANNNDRTYWELLSGGEPLSQGAGSYVVRNGENLEVRWSKY

SEQ ID NO: 28 - DNA, 1577 bp, Homo sapiens
ggctgaggcaacctgaaggaggagctctcattaccttctgcccatcacttaataaatagccagccaattcatcaacattctggtacactgttggagagatgagacagtcacaccagctgcccctagtggggctcttactgttttcttttattccaagccaactatgcgagatttgtgaggtaagtgaagaaaactacatccgcctaaaacctctgttgaatacaatgatccagtcaaactataacaggggaaccagcgctgtcaatgttgtgttgtccctcaaacttgttggaatccagatccaaaccctgatgcaaaagatgatccaacaaatcaaatacaatgtgaaaagcagattgtcagatgtaagctcgggagagcttgccttgattatactggctttgggagtatgtcgtaacgctgaggaaaacttaatatatgattaccacctgatcgacaagctagaaaataaatcccaagcagaaattgaaaatatggaagcacacaatggcactcccctgactaactactaccagctcagcctggacgttttggccttgtgtctgttcaatgggaactactcaaccgccgaagttgtcaaccacttcactcctgaaaataaaaactattattttggtagccagttctcagtagatactggtgcaatggctgtcctggctctgacctgtgtgaagaagagtctaataaatgggcagatcaaagcagatgaaggcagtttaaagaacatcagtatttatacaaagtcactggtagaaaagattctgtctgagaaaaaagaaaatggtctcattggaaacacatttagcacaggagaagccatgcaggccctctttgtatcatcagactattataatgaaaatgactggaattgccaacaaactctgaatacagtgctcacggaaatttctcaaggagcattcagcaatccaaacgctgcagcccaggtcttacctgccctgatgggaaagaccttcttggatattaacaaagactcttcttgcgtctctgcttcaggtaacttcaacatctccgctgatgagcctataactgtgacacctcctgactcacaatcatatatctccgtcaattactctgtgagaatcaatgaaacatatttcaccaatgtcactgtgctaaatggttctgtcttcctcagtgtgatggagaaagcccagaaaatgaatgatactatatttggtttcacaatggaggagcgctcatgggggccctatatcacctgtattcagggcctatgtgccaacaataatgacagaacctactgggaacttctgagtggaggcgaaccactgagccaaggagctggtagttacgttgtccgcaatggagaaaacttggaggttcgctggagcaaatactaataagcccaaactttcctcagctgcataaaatccatttgcagtggagttccatgtttattgtccttatgccttcttcttcatttatcccagtacgagcaggagagttaataacctccccttctctctctacatgttcaataaaagttgttgaaagattaacaactataaaaaaaaaaa

SEQ ID NO: 29 - protein, 432 aa, Homo sapiens
RQSHQLPLVGLLLFSFIPSQLCEICEVSEENYIRLKPLLNTMIQSNYNRGTSAVNVVLSLKLVGIQIQTLMQKMIQQIKYNVKSRLSDVSSGELALIILALGVCRNAEENLIYDYHLIDKLENKSQAEIENMEAHNGTPLTNYYQLSLDVLALCLFNGNYSTAEVVNHFTPENKNYYFGSQFSVDTGAMAVLALTCVKKSLINGQIKADEGSLKNISIYTKSLVEKILSEKKENGLIGNTFSTGEAMQALFVSSDYYNENDWNCQQTLNTVLTEISQGAFSNPNAAAQVLPALMGKTFLYINKDSSCVSASGNFNISADEPITVTPPDSQSYISVNYSVRINETYFTNVTVLNGSVFLSVMEKAQKMNDTIFGFTMEERSWGPYITCIQGLCANNNDRTYWELLSGGEPLSQGAGSYVVRNGENLEVRWSKY